SEQUENCE LISTING (28 sequences)

SEQ ID NO: 1 - DNA, 24 bp, Artificial Sequence
  Description of Artificial Sequence Synthetic primer
ctgaatgcccagacagctccaagc

SEQ ID NO: 2 - DNA, 21 bp, Artificial Sequence
  Description of Artificial Sequence Synthetic primer
tcactgatacggagctgaggc

SEQ ID NO: 3 - DNA, 22 bp, Artificial Sequence
  Description of Artificial Sequence Synthetic primer
ccttgcagcctagaaattcagt

SEQ ID NO: 4 - DNA, 22 bp, Artificial Sequence
  Description of Artificial Sequence Synthetic primer
gcctcaagtcgcttccaacctc

SEQ ID NO: 5 - DNA, 24 bp, Artificial Sequence
  Description of Artificial Sequence Synthetic primer
cattatgataaaatggagagagat

SEQ ID NO: 6 - DNA, 24 bp, Artificial Sequence
  Description of Artificial Sequence Synthetic primer
aaggtggagagagacaaaggattc

SEQ ID NO: 7 - DNA, 21 bp, Artificial Sequence
  Description of Artificial Sequence Synthetic primer
agaaaggaaacctgcctggtt

SEQ ID NO: 8 - DNA, 21 bp, Artificial Sequence
  Description of Artificial Sequence Synthetic primer
ctctcactgtgacatctgccc

SEQ ID NO: 9 - DNA, 22 bp, Artificial Sequence
  Description of Artificial Sequence Synthetic primer
tacagggtctcacggaagaagc

SEQ ID NO: 10 - DNA, 21 bp, Artificial Sequence
  Description of Artificial Sequence Synthetic primer
cattactcatatgtcgctgac

SEQ ID NO: 11 - DNA, 21 bp, Artificial Sequence
  Description of Artificial Sequence Synthetic primer
cattattcatatggtgctggc

SEQ ID NO: 12 - DNA, 22 bp, Artificial Sequence
  Description of Artificial Sequence Synthetic primer
tgctggcaaccttcgaatagga

SEQ ID NO: 13 - DNA, 24 bp, Artificial Sequence
  Description of Artificial Sequence Synthetic primer
tctctctacattggctctgcaggc

SEQ ID NO: 14 - DNA, 23 bp, Artificial Sequence
  Description of Artificial Sequence Synthetic primer
atcaagtctgtagagccggagga

SEQ ID NO: 15 - DNA, 25 bp, Artificial Sequence
  Description of Artificial Sequence Synthetic primer
gcactcaactctgaagatccagagc

SEQ ID NO: 16 - DNA, 22 bp, Artificial Sequence
  Description of Artificial Sequence Synthetic primer
gatggtggggctttcaaggatc

SEQ ID NO: 17 - DNA, 24 bp, Artificial Sequence
  Description of Artificial Sequence Synthetic primer
aggcctaaaggaactaactcccac

SEQ ID NO: 18 - DNA, 22 bp, Artificial Sequence
  Description of Artificial Sequence Synthetic primer
acgaccaattcatcctaagcac

SEQ ID NO: 19 - DNA, 24 bp, Artificial Sequence
  Description of Artificial Sequence Synthetic primer
cccatcagtcatcccaacttatcc

SEQ ID NO: 20 - DNA, 21 bp, Artificial Sequence
  Description of Artificial Sequence Synthetic primer
cactctgaaaatccaacccac

SEQ ID NO: 21 - DNA, 20 bp, Artificial Sequence
  Description of Artificial Sequence Synthetic primer
agtgttcctcgaactcacag

SEQ ID NO: 22 - DNA, 24 bp, Artificial Sequence
  Description of Artificial Sequence Synthetic primer
cagccggccaaacctaacattctc

SEQ ID NO: 23 - DNA, 21 bp, Artificial Sequence
  Description of Artificial Sequence Synthetic primer
ctgctaagaaaccatgtacca

SEQ ID NO: 24 - DNA, 21 bp, Artificial Sequence
  Description of Artificial Sequence Synthetic primer
tctgcagcctgggaatcagaa

SEQ ID NO: 25 - DNA, 21 bp, Artificial Sequence
  Description of Artificial Sequence Synthetic primer
gccagaaggtagcagagaccc

SEQ ID NO: 26 - DNA, 21 bp, Artificial Sequence
  Description of Artificial Sequence Synthetic primer
gagaaatgtgactccacccaa

SEQ ID NO: 27 - DNA, 21 bp, Artificial Sequence
  Description of Artificial Sequence Synthetic primer
  5′-FAM
ttgggtggagtcacatttctc

SEQ ID NO: 28 - DNA, 21 bp, Artificial Sequence
  Description of Artificial Sequence Synthetic primer
  5′-HEX
ttgggtggagtcacatttctc

(19) United States
(12) Patent Application Publication
(10) Pub. No.: US 2021/0179714 A1
(43) Pub. Date: Jun. 17, 2021

(54) Inhibition of CTLA-4 and/or PD-1 For Regulation of T Cells

(71) Applicant: MEMORIAL SLOAN KETTERING CANCER CENTER, New York, NY (US)

(72) Inventors: Jedd WOLCHOK, New York, NY (US); Roberta ZAPPASODI, New York, NY (US); Taha MERGHOUB, New York, NY (US)

(21) Appl. No.: 16/761,784

(22) PCT Filed: Nov. 6, 2018
(86) PCT No.: PCT/US2018/059337

§ 371 Date: May 5, 2020

Related U.S. Application Data
(60) Provisional application No. 62/582,416, filed on Nov. 7, 2017.

(51) Int. Cl. C07K 16/28; A61P 35/00

(52) U.S. Cl. CPC C07K 16/2818; A61K 2039/507; A61P 35/00

(57) ABSTRACT

Increases in CD4⁺Foxp3⁻PD-I^{hi} T cells (4PD1^{hi}) in tumor-bearing hosts after CTLA-4 blockade show that these cells constitute an unconventional T-cell inhibitory subset with T_{FH}-like features, which can affect the outcome of cancer immunotherapy. Evidence is provided that anti-PD-1/PD-L1 antibodies arc a viable option to control these cells. Furthermore, treating cancer by administering immune checkpoint blockade therapy and monitoring circulating 4PD1^{hi} provides a more precise or personalized design of combination immunotherapies.

### CROSS-REFERENCE TO RELATED APPLICATIONS

This application claims the benefit of priority of U.S. Provisional Patent Application No. 62/582,416, filed on Nov. 7, 2017, the entire contents of which are incorporated by reference.

### STATEMENT OF GOVERNMENT SUPPORT

This invention was made with government support under CA008748 awarded by the National Institutes of Health. The government has certain rights in the invention.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### BACKGROUND

Cytotoxic T-lymphocyte-associated protein-4 (CTLA-4) and programmed cell death protein-1 (PD-1) are the best-characterized immune co-inhibitory receptors that have been successfully exploited as therapeutic targets to promote and reinvigorate immune responses against cancer. Both molecules are induced on T cells upon T-cell receptor (TCR) signaling activation, but with different kinetics. CTLA-4 is usually up-regulated during the initial stage of naïve T-cell activation, and competes with CD28 for the same ligands (CD86 and CD80) expressed on antigen presenting cells (APCs), thus limiting excessive T-cell priming (Fife and Bluestone, 2008; Pentcheva-Hoang et al., 2004). CTLA-4 is also constitutively expressed at high levels on regulatory T cells (T_{regs}), and constitutes one of their immunosuppressive mechanisms (Wing et al., 2008). PD-1 is generally induced during the later phases of an immune response, thus controlling previously activated T cells, typically at the effector sites of immune responses. PD-1 is considered the prototype marker of T-cell exhaustion (Fife and Bluestone, 2008; Keir et al., 2008). The CTLA-4 and PD-1 immune checkpoints are particularly deregulated in tumor-bearing hosts, where chronic ineffective immune responses usually predominate and result in T-cell exhaustion and T_{reg} induction (Wing et al., 2008). These observations provided the rationale for developing strategies to inhibit CTLA-4 and PD-1 as new cancer immunotherapy modalities (Dong et al., 2002; Iwai et al., 2002; Leach et al., 1996; Strome et al., 2003).

Blockade of these two immune checkpoints with specific antibodies (anti-CTLA-4 and anti-PD-1) has now become a standard of care for metastatic melanoma, producing tumor regression in about 20-45% of patients when used as monotherapies, and in up to 60% of the cases when used in combination (Hodi et al., 2010; Larkin et al., 2015; Robert et al., 2015; Weber et al., 2015). PD-1 blockade has more recently achieved impressive clinical results in chemotherapy-refractory advanced non-small cell lung cancer (NSCLC) patients, where it is currently being investigated in combination with CTLA-4 blockade (Hellmann et al., 2016; Lutzky et al., 2014).

The clinical experience accumulated thus far reveals differing activity profiles of CTLA-4 and PD-1 blockade, which can eventually complement each other, as indicated by results from their use in combination (Larkin et al., 2015; Postow et al., 2015; Wolchok et al., 2013). Given the dominant immune evasion associated with programmed death-ligand 1 (PD-L1) overexpression in tumors, PD-1 pathway blockade yields superior therapeutic activity (Larkin et al., 2015; Postow et al., 2015; Robert et al., 2015). However, anti-PD-1 as a monotherapy or in combination with anti-CTLA-4 can produce notable clinical benefit even in patients with tumors that express very low levels of PD-L1 (Brahmer et al., 2015; Larkin et al., 2015), indicating that multiple non-redundant effects on the immune system may also occur.

Despite these successes, immune checkpoint blockade still does not benefit a significant proportion of patients with metastatic cancer, and poses a potentially high risk for developing severe immune-related toxicities, in particular when anti-CTLA-4 and anti-PD-1 are combined (Friedman et al., 2016). In addition, except for tumor-associated PD-L1 expression, which can help enrich for patients more likely to respond to PD-1 pathway blockade (Topalian et al., 2012), there are no validated biomarkers guiding selection of optimal checkpoint blockade combinations across different tumor types. This underscores the need to better understand the biologic activity of anti-CTLA-4 and anti-PD-1 for more precise utilization of these strategies.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

We show herein that a specific population of cells designated “4PD1^{hi}” (defined below) have a negative impact on anti-tumor immunity: (i) intra-tumor 4PD1^{hi} accumulation occurs as a function of tumor progression, and (ii) tumor-associated and peripheral 4PD1^{hi} from mice and humans limit effector T-cell (T_{eff}) functions. In addition, we show that anti-CTLA-4 consistently promotes increases in 4PD1^{hi}, while PD-1 blockade mitigates this effect and counteracts 4PD1^{hi} inhibitory function. The clinical relevance of this cell population is confirmed by our finding that persistence of high 4PD1^{hi} levels is a negative prognostic factor in patients treated with PD-1 blockade.

Collectively, these results reveal the negative impact on T-cell responses of 4PD1^{hi}, which are induced by CTLA-4 blockade, presumably as a consequence of heightened T-cell priming (Sage et al., 2014b; Wing et al., 2014), and can be counteracted quantitatively and functionally by anti-PD-1. Our findings illustrate a novel mechanism of response/resistance to checkpoint blockade therapy. Since modulation of inhibitory 4PD1^{hi} is reliably detected in peripheral blood (PB), prospective assessment of circulating 4PD1^{hi} during checkpoint blockade treatment can provide important information for regimen and treatment optimization.

Accordingly, the invention provides a method of treating cancer in a patient undergoing immune checkpoint blockade (ICB) therapy, the method comprising: (a) measuring 4PD1^{hi} cell frequency in a blood sample from the patient at least about three weeks after a dose of ICB therapy comprising a dosage of at least one of a PD-1 inhibitor and a CTLA-4 inhibitor; and (b) administering to the patient another dose of ICB therapy, wherein the dosages of the PD-1 inhibitor and the CTLA-4 inhibitor are adjusted based on the 4PD1^{hi} cell frequency. In some instances, the 4PD1^{hi} cell frequency in step (b) is compared to the 4PD1^{hi} cell frequency in a blood sample from the patient prior to the dose of ICB therapy in step (a), i.e., a baseline 4PD1^{hi} cell frequency.

In a particular embodiment, the dosage of the PD-1 inhibitor is increased and/or the dosage of the CTLA-4 inhibitor is decreased if the 4PD1^{hi} cell frequency is high. In another embodiment, the dosage of the PD-1 inhibitor can be decreased and/or the dosage of the CTLA-4 inhibitor can be increased if the 4PD1^{hi} cell frequency is low.

The invention also provides a method for predicting a response to ICB therapy in a cancer patient and treating the cancer patient with ICB therapy, the method comprising: (a) measuring 4PD1^{hi} cell frequency in a blood sample from the cancer patient; (b) classifying the cancer patient as susceptible to respond to ICB therapy wherein the 4PD1^{hi} cell frequency is low or classifying the cancer patient as resistant to ICB therapy wherein the 4PD1^{hi} cell frequency is high; and (c) administering to the cancer patient: a higher dosage of a PD-1 inhibitor and/or a lower dosage of a CTLA-4 inhibitor wherein the patient is resistant to ICB therapy.

Further provided is an ex vivo method for determining whether a cancer patient is susceptible to ICB therapy comprising a CTLA-4 inhibitor, the method comprising measuring 4PD1^{hi} cell frequency in a blood sample from the cancer patient, wherein a low 4PD1^{hi} cell frequency indicates that the patient is susceptible to ICB therapy comprising a CTLA-4 inhibitor and wherein a high 4PD1^{hi} cell frequency indicates that the patent is resistant to ICB therapy comprising a CTLA-4 inhibitor.

In addition, a method is provided for in vitro prediction of the probability of a cancer patient responding to ICB therapy comprising a CTLA-4 inhibitor, the method comprising: (a) determining the frequency of 4PD1^{hi} cells in a blood sample from the cancer patient; and (b) comparing the frequency of 4PD1^{hi} cells determined in step (a) with a reference frequency of 4PD1^{hi} cells obtained from cancer patients who have responded to ICB therapy comprising a CTLA-4; wherein, if the frequency of 4PD1^{hi} cells determined in step (a) is the same as or lower than the reference frequency, it is predicted that the cancer patient will respond to ICB therapy comprising CTLA-4.

One embodiment of the invention is the use of a composition for predicting or monitoring a response to ICB therapy in a cancer patient, the composition comprising 4PD1^{hi} cells in an ex vivo blood sample from the cancer patient.

In one aspect, the invention provides the use of the measurement of the frequency of 4PD1^{hi} cells in vitro in a blood sample from a patient as a biomarker for success of ICB therapy in a cancer patient.

In certain embodiments, ICB therapy comprises a PD-1 inhibitor and/or a CTLA-4 inhibitor. In some embodiments, the ICB therapy comprises a PD-1 inhibitor and a CTLA-4 inhibitor. In some embodiments, the ICB therapy comprises a PD-1 inhibitor. In some embodiments, the ICB therapy comprises a CTLA-4 inhibitor. In some embodiments, the PD-1 inhibitor is an antibody. In some embodiments, the PD-1 inhibitor is selected from the group consisting of nivolumab, pembrolizumab, pidilizumab, and REGN2810. In some embodiments, the PD-1 inhibitor is a PD-L1 inhibitor selected from the group consisting of atezolizumab, avelumab, durvalumab, and BMS-936559. In some embodiments, the CTLA-4 inhibitor is an antibody. In some embodiments, the CTLA-4 inhibitor is selected from the group consisting of ipilimumab and tremelimumab.

In some embodiments of the invention, the patient undergoing ICB therapy is administered a B cell lymphoma 6 (BCL6) inhibitor. In some such embodiments the BCL6 inhibitor is administered to the patient after measuring the 4PD1^{hi} cell frequency in a blood sample from the patient. In some such embodiments, the BCL6 inhibitor is administered to the patient concurrently with administering a dose of ICB therapy to the patient. In some such embodiments the BCL6 inhibitor is administered to the patient after measuring the 4PD1^{hi} cell frequency in a blood sample from the patient and concurrently with administering a dose of ICB therapy to the patient.

In some embodiments, 4PD1^{hi} cell frequency is measured in a blood sample from the patient prior to a first dose of ICB therapy.

In some embodiments, 4PD1^{hi} cell frequency is measured using immunohistochemistry (IHC), such as immunofluorescence staining or multiplex IHC. In some embodiments, 4PD1^{hi} cell frequency is measured using flow cytometry, such as fluorescence-activated cell sorting (FACS). In some embodiments, 4PD1^{hi} cell frequency is measured using a gene expression signature.

### BRIEF DESCRIPTION OF THE FIGURES

FIG. 1A-1C show that 4PD1^{hi} cells accumulate intratumorally in mice and humans. Mice were injected with 0.25×10⁵, 0.5×10⁵, 1×10⁵, or 2×10⁵ B16 cells (5 mice/group). Two weeks later, 4PD1^{hi} and T_{regs} were analyzed in spleen (SP), tumor-draining lymph nodes (DLNs), and tumor (TM). 4PD1^{hi} and T_{reg} frequencies in these anatomic locations in comparison with spleens from naïve mice (SP naïve ) (FIG. 1A), and correlation with tumor burden of intra-tumor 4PD1^{hi} and T_{reg} frequencies and the indicated intra-tumor T-cell ratios (FIG. 1B). P values and Pearson r correlation coefficients indicate statistically significant results. FIG. 1C shows 4PD1^{hi}/CD4% in healthy donors' PB (HD, n=7), in advanced melanoma patients' PB (n=47) and malignant lesions (TM, n=10), and in NSCLC patients' PB (n=51) and malignant lesions (TM, n=10). FIG. 1C also shows representative plots of Foxp3 and PD-1 expression in live single CD4⁺CD45⁺ cells, and CD25 expression in 4PD1^{hi}, T_{regs}, and conventional PD-1⁻Foxp3⁻CD4⁺ T cells (“4PD1^{neg}”) from the indicated donors' and patients' samples. Unpaired t test: *p<0.05, **p<0.01, ***p<0.001, ****p<0.0001.

FIG. 2A-2E show that 4PD1^{hi} cells accumulate at the tumor site with tumor progression and are antigen-experienced T cells. FIG. 2A shows correlation of tumor burden with intra-tumor 4PD1hi frequency and CD8/4PD1hi ratio in mice injected with the same amount of B16 cells (10⁵ cells). P values and Pearson r correlation coefficients are included in the graphs. FIG. 2B shows frequency of 4PD1^{hi} and T_{regs} in spleen (SP), tumor-draining lymph nodes (DLNs), and tumor (TM), and ratios between the indicated T-cell subsets at the tumor site in Grm1-TG mice at an early (3 months old; mean±SEM of 3 mice) or advanced (6 months old; mean±SEM of 5 mice) stage of melanoma development. FIG. 2C shows FACS analysis of Ki67 and FIG. 2D shows CD44 and CD62L expression in the indicated cell subsets and anatomic locations in naïve and B16-bearing mice, as in FIG. 1A. FIG. 2E shows examples of oligoclonal CDR3 spectratypes (TCRBV1, TCRBV2, TCRBV10, TCRBV11, and TCRBV15) in 4PD1^{hi}, T_{regs}, and 4PD1^{neg} sorted from tumors (TM) of B16-bearing Foxp3-GFP transgenic mice. The same analysis in 4PD1^{neg} isolated from naïve spleens (SP) is reported as control. Unpaired t test: *p<0.05, **p<0.01, ***p<0.001, ****p<0.0001.

FIG. 3A-3E show that mouse 4PD1^{hi} cells limit T-cell effector functions. FIG. 3A shows 4PD1^{neg}, 4PD1^{hi}, or Conventional PD-F Foxp3⁺T_{regs}, FACS-sorted from spleens of naïve Foxp3-GFP transgenic mice (CD45.1⁻) as indicated, and tested in in vitro suppression assays with αCD3-stimulated CTV-labeled target T cells from CD45.1⁺ congenic mice. FIG. 3B shows representative FACS analysis of CTV dilution, CD44, and CD25 co-expression in total CD45.1⁺CD4⁺ target T cells. FIG. 3C shows quantification of IFN-γ, TNF-α, and IL-2 in supernatants from the same cultures (ratio 1:1). FIG. 3D shows Foxp3, CD25, and PD-1 expression in “suppressor” CD45.1⁻CD4⁺ T-cell subsets from the same cultures (ratio 1:1). Data are the mean±SD of duplicate cultures. FIG. 3E shows in vivo T-cell inhibitory activity of 4PD1^{hi} compared with T_{regs}, FACS-sorted from B16-bearing Foxp3-GFP transgenic mice, co-transferred with CFSE-labeled Pmel/gp100-TCR-specific CD8⁺ T cells (Pmels) (1:1 ratio) into irradiated CD45.1⁺ recipients, and stimulated in vivo with irradiated B16 cells the day after transfer. Proliferation (CFSE dilution) and activation (CD44 and CD25 expression) of CD45.1⁻Thy1.1⁺CD8⁺ Pmels were recovered in recipient spleens. 2-way ANOVA or unpaired t test: *p<0.05, **p<0.01, ***p<0.001, ****p<0.0001.

FIG. 4A-4C show that mouse 4PD1^{hi} cells limit T-cell effector functions. 4PD1^{hi}, 4PD1^{neg}, and conventional T_{regs} were FACS-sorted from spleens of naïve non-tumor-bearing Foxp3-GFP transgenic mice and tested in suppression assays as described in FIG. 3A. Data show the results of two additional independent experiments using as target CTV-labeled CD45.1⁺ CD8⁺ (FIG. 4A) or CD4⁺ (FIG. 4B) T cells. Proliferation and activation of target cells were measured by FACS analysis of CTV dilution and CD44/CD25 co-expression, respectively, after 48 (FIG. 4A) and 72 (FIG. 4B) hours in culture. Representative FACS plots and culture pictures show results from co-cultures at 1:1 ratio. FIG. 4C shows the proliferation capacity of spleen-derived 4PD1^{neg}, 4PD1^{hi}, and T_{regs} after 72-hour stimulation with anti-CD3/CD28 coated beads.

FIG. 5A-5C show that human 4PD1^{hi} cells limit T-cell effector functions. FIG. 5A (left panels) shows representative plots of the gating strategy to sort human 4PD1^{hi}, total T_{regs}, and 4PD1^{neg} based on PD-1 and CD25 expression in live CD4⁺ T cells; Foxp3 expression was confined to CD25-positively gated T_{regs}. FIG. 5A (left middle panels) shows proliferation (CTV^{low}) and activation (CD25 MFI) of autologous target CD4⁺ T cells co-cultured with the indicated donor-derived circulating CD4⁺ T-cell subsets at 1:1 ratio. FIG. 5A (right middle and right panels) shows unsupervised hierarchical clustering and related heatmap of production of the indicated cytokines in supernatants from the same cultures. Inhibitory effects of human tumor-infiltrating 4PD1^{hi} compared to T_{regs} and 4PD1^{neg} on autologous CD4⁺ TILs (FIG. 5B) or donor-derived allogeneic circulating CD8⁺ T cells (FIG. 5C) (1:1 ratio) are shown. Proliferation (CTV^{low}) of target T cells and cytokine production in the same cultures are shown. Data are the mean±SD of 2-6 replicate cultures/condition (unpaired t test: *p<0.05, **p<0.01, ***p<0.001, ****p<0.0001).

FIG. 6A-6C show that Human 4PD1^{hi} cells limit T-cell effector functions. FIG. 6A shows effects of circulating 4PD1^{hi} in comparison with T_{regs} and 4PD1^{neg} from 4 additional healthy donors on proliferation (CTV^{low} %) and activation (CD25 MFI) of autologous target CD4⁺ T cells (1:1 ratio), tested in 4 independent experiments. FIG. 6B shows the phenotype of donor-derived 4PD1^{hi}, T_{regs}, and 4PD1^{neg} after in vitro culture with target CD4⁺ T cells from one representative experiment. FIG. 6C shows activation of target CD4⁺ T cells and phenotypic analysis of “suppressor” CD4⁺ T-cell subsets from in vitro suppression assays with human TILs shown in FIG. 5B. Data are average±SD of 2-6 replicate cultures/condition; unpaired t test: *p<0.05, **p<0.01, ***p<0.001, ****p<0.0001.

FIG. 7A-7B show analysis of cross-reactivity between therapeutic and detection anti-human and anti-mouse PD-1 Abs. FIG. 7A shows peripheral blood mononuclear cells (PBMC) from a healthy donor and a nivolumab- (top panel) or a prembrolizumab-treated patient (bottom panel), co-stained with a PE-labeled anti-human IgG4 (to detect therapeutic anti-PD-1 mAbs) and the FITC-labeled anti-human PD-1 used in flow cytometry analyses (MIH4) or the matched isotype IgG. Plots represent the overlay of live single CD4⁺ T cells between donor (black) and patient (gray) samples. FIG. 7B shows PD-1 expression in mouse splenocytes pre-incubated with or without the therapeutic anti-mouse PD-1 monoclonal Ab (mAb) used in this study (RMP1-14), as revealed by FACS with the APC-conjugated anti-PD-1 mAb RMP1-30 (top panel), or with the rabbit anti-PD-1 polyclonal Ab used in immunofluorescent staining, followed by FITC-labeled secondary Ab (bottom panel).

FIG. 8A-8H show modulation of 4PD1^{hi} cells and efficacy of immune checkpoint blockade. FIG. 8A shows modulation of circulating 4PD1^{hi}/CD4% relative to baseline at the indicated time points in advanced NSCLC patients during treatment with nivo3 (nivolumab 3 mg/kg, q2 wks, n=10), nivo3+ipi1 (nivolumab 3 mg/kg+ipilimumab 1 mg/kg, q3 wks, q6 wks+q2 wks, or q12 wks+q2 wks, n=21), nivo1+ipi1 (nivolumab 1 mg/kg+ipilimumab 1 mg/kg, q3 wks, or q6 wks, n=11), or nivo1+ipi3 (nivolumab 1 mg/kg+ipilimumab 3 mg/kg, q3 wks, n=8). Comparison between nivo3 and nivo1+ipi1 or between nivo3 and nivo1+ipi3 was by 2-way ANOVA with Bonferroni's multiple comparisons test. FIG. 8B shows modulation of circulating 4PD1^{hi}/CD4% in B16-melanoma-bearing mice treated with αCTLA-4 monotherapy (100 μg or 300 μg/cycle, 7-10 mice/group, average±SEM) relative to naïve mice (5 mice) (2-way ANOVA with Bonferroni's multiple comparisons test). FIG. 8C shows pairwise comparison of 4PD1^{hi}/CD4% at the indicated time points relative to baseline in advanced melanoma patients during ipilimumab (ipi, 3 mg/kg, q3 wks; n=47) or pembrolizumab treatment (pembro, 2 mg/kg or 10 mg/kg, q3 wks; n=52). FIG. 8D shows average±SEM tumor diameter (left panel; 10 mice/group, 2-way ANOVA with Bonferroni's multiple comparisons test) and Kaplan-Meier tumor-free survival curves (right panel; pooled data from 3 independent experiments, 30 mice/group, log-rank test; number of tumor-free mice approximately 100 days after tumor implantation is reported for each group) from B16-bearing mice vaccinated with VRP-TRP2 and treated with anti-CTLA-4 and/or anti-PD-1 or the isotype-matched IgG controls, as indicated with arrows. FIG. 8E shows frequency of intra-tumor 4PD1^{hi} and Foxp3⁺ T_{regs} one day after treatment completion (9-10 mice/group, average±SEM, unpaired t test). FIG. 8F shows circulating 4PD1^{hi} and T_{reg} frequency (top) and modulation relative to baseline (bottom) in advanced melanoma patients during pembrolizumab treatment (2 mg/kg, q3 wks; n=18) (Huang et al., 2017). FIG. 8G shows that >2.2% 4PD1^{hi} (as a percentage of CD4+ cells) after treatment with PD-1 blockade portends an unfavorable outcome in melanoma patients administered pembrolizumab. A higher dose of pembrolizumab is more efficient at down-regulating 4PD1^{hi} (bottom panel). FIG. 8H shows that a 51%or less reduction in 4PD1^{hi} cell frequency after treatment with PD-1 blockade portends an unfavorable outcome in melanoma patients administered pembrolizumab. *p<0.05, **p<0.01, ***p<0.001, ****p<0.0001.

FIG. 9A-9C show anti-CTLA-4-dose- and tumor-dependent modulation of 4PD1^{hi} cell frequency. In FIG. 9A, B16-melanoma-bearing C57BL/6J mice were treated with anti-CTLA-4 monotherapy (100 μg or 300 μg) or isotype-matched control IgG (300 μg) as shown in FIG. 8B. One day after treatment completion, tumor biopsies were subjected to immunofluorescent staining of CD4 (AlexaFluor488), Foxp3 (AlexaFluor568), and PD-1 (AlexaFluor647). Representative staining of 4PD1^{hi} cells (indicated by arrows; scale bar=50 μm; 40× original magnification) and quantification of 4PD1^{hi} cells in 3 tumors/group. In FIG. 9B, non-tumor-bearing C57BL/6J mice were treated with 4 courses of anti-CTLA-4 (100 μg or 300 μg) or the matched isotype IgG (300 μg). One day after treatment completion, 4PD1^{hi}/CD4% was measured in PB and spleen by FACS. In FIG. 9C, TUBO-breast-carcinoma-bearing or naïve Balb/c mice were treated with 4 courses of the indicated amount of anti-CTLA-4 or the matched isotype IgG. 4PD1^{hi} cells were monitored in tumor and spleen after the 2^{nd} (C2) and the 4^{th} (C4) administration (TUBO-bearing mice, mean±SEM of 5 mice/group) or at the end of treatment (naïve mice, mean±SEM of 4-5 mice/group). Unpaired t test: *p<0.05, **p<0.01, ***p<0.001.

FIG. 10A-10B show effects of T_{regs} and 4PD1^{hi} cells in a 3D killing assay. FIG. 10A shows inhibition of CD8⁺ T-cell-mediated tumor killing by suppressive T cells in a 3D killing assay. Percent killed B16 cells in co-cultures with tumor-specific CD8⁺ T cells (tumor-antigen specific shown in top graph; CD8 TILs shown in bottom graph) and tumor-derived T_{regs} or 4PD1^{hi} cells are shown in comparison with 4PD1^{neg} (average±SD of 3-6 replicate cultures/condition, unpaired t test: ***p<0.001, ****p<0.0001). FIG. 10B shows representative FACS plots of the indicated markers in CD8⁺ TILs and IFNγ-pre-treated B16 used in 3D killing assays. B16 cells employed in 3D killing assays were pre-treated with IFNγ to up-regulate MHC-I (H-2Kb) and MHC-II (I-E/I-A) and to be recognized by both CD8⁺ and CD4⁺ T cells in culture. Ag=antigen.

FIG. 11A-11C show that PD-1/PD-L1 blockade counteracts 4PD1^{hi} cell inhibitory function. 4PD1^{neg} and 4PD1^{hi} cells FACS-sorted from tumors of untreated B16-bearing Foxp3-GFP mice were co-cultured with FACS-sorted CD8⁺ TILs (CD8:CD4=0.5×10⁵:0.1×10⁵, suboptimal conditions) and target B16 cells in 3D killing assays. FIG. 11A shows the percent of killed B16 in co-cultures treated with anti-PD-1, anti-PD-L1, or matched isotype IgGs, relative to B16 cultured alone (mean±SD of 2-3 replicate cultures/condition). FIG. 11B shows the percent of killed B16 in culture with FACS-sorted CD8⁺ TILs and anti-PD-1- or anti-PD-L1-pre-treated 4PD1^{hi} or 4PD1^{neg}, relative to B16 cultured alone (top panel; mean±SD of 2-3 replicate cultures/condition); and PD-L1 expression in 4PD1^{hi} compared with 4PD1^{neg} and CD8⁺ T cells in spleen, tumor-draining lymph nodes (DLNs), and tumor from B16-bearing mice (bottom panel; n=10). FIG. 11C shows quantification, in human NSCLC-derived 4PD1^{hi}, T_{regs}, and 4PD1^{neg} pre-treated with anti-PD-1 or control isotype IgG and cultured with stimulated autologous CD8⁺ TILs, of the indicated pro-inflammatory cytokines (mean±SD of 2-6 replicate cultures/condition). Unpaired t test: *p<0.05, **p<0.01, ***p<0.001, ****p<0.0001.

FIG. 12A-12B show differential gene expression profiles of mouse and human 4PD1^{hi}. FIG. 12A shows unsupervised hierarchical clustering with the related heatmap (left panel) and principal component analysis (right panel) of variably expressed genes (sds>0.04, n=12,083) in mouse splenic 4PD1^{neg}, 4PD1^{hi} cells, and conventional T_{regs}, functionally validated in 3 independent experiments (FIG. 3B, FIG. 4A-4B). FIG. 12B shows unsupervised hierarchical clustering with the related heatmap (left panel) and principal component analysis (right panel) of differentially expressed genes (adjusted p value<0.05, n=2,059) in donor-derived 4PD1^{neg}, 4PD1^{hi} cells, and T_{regs}, functionally validated in 5 independent experiments (FIG. 5A and FIG. 6A).

FIG. 13A-13F show that mouse and human 4PD1^{hi} cells are a distinct CD4⁺ T-cell subset with a T_{FH}-like phenotype. Unsupervised hierarchical clustering with the related heatmap and single-sample gene set enrichment analysis (ssGSEA) scores of T_{FH}-associated genes in gene expression datasets from mouse splenic (FIG. 13A) and donor-derived (FIG. 13B) 4PD1^{neg}, 4PD1^{hi} cells, and T_{regs} functionally validated, respectively, in 3 and 5 independent experiments (FIG. 3B and FIG. 4; FIG. 5A and FIG. 6A) are shown. *p=0.03125 Wilcoxon matched-pairs signed-rank test. FIG. 13C shows 4PD1^{hi} cell frequencies in tumors from B16-bearing Batf KO or WT mice treated with anti-CTLA-4 or control isotype IgG (100 μg×4), as assessed by FACS (mean±SEM of 6-10 mice/group, unpaired t test) or immunofluorescence staining (IF; mean±SEM of 3 mice/group, unpaired t test) one day after treatment completion. FIG. 13D shows CD86 expression on circulating B cells (live single B220⁺CD45⁺) from B16-melanoma-bearing mice treated with 4 courses of αCTLA-4 (100 μg) or the matched isotype IgG (left; 9-10 mice/group, unpaired t test), and on circulating B cells (live single CD19⁺CD45⁺) before and during ipilimumab treatment (ipi) in metastatic melanoma patients (right; 3 mg/kg, q3 wks, n=16, paired t test). 4PD1^{hi}, memory CD4⁺ T cells (CD44^{hi}PD-1-Foxp3⁻CD4⁺ T cells, Tₘₑₘ) and Foxp3⁺ T_{regs} were sorted from tumors (FIG. 13E) and spleens (FIG. 13F) of B16-bearing Foxp3-GFP mice treated with 4 courses of αCTLA-4 and tested in standard suppression assays with CTV-labeled target T cells from naïve CD45.1⁺ congenic mice at the indicated effector:target ratios. Proliferation (CTV^{low}) and activation (CD25⁺CD44⁺) of target T cells were quantified in each condition (mean±SD of 3 replicate cultures/condition). Representative plots show the gating strategy used to sort 4PD1^{hi}, Tₘₑₘ and T_{regs} and baseline CD44 expression in the 3 sorted cell subsets. 2-way ANOVA with Bonferroni's multiple comparisons test and unpaired t test: *p<0.05, **p<0.01, ***p<0.001, ****p<0.0001.

FIG. 14A-14F show T_{FH}-like phenotype in 4PD1^{hi} cells from naïve and tumor-bearing mice. Gene Set Enrichment Analysis (GSEA) of gene signatures from various CD4⁺ T-cell subsets in 4PD1^{hi} and Treg gene expression data sets generated in our study. Gene sets for T_{H}1, T_{H}2, T_{H}17, iTREG, and nTREG are from GSE14308, gene sets for EXH, MEM, EFF from GSE30431 and T_{FH} from GSE85316, and are all relative to nave T cells. Tr1 gene set is from GSE92940 and relative to Th0 cells. GSEA v2.2.4 was run with the following parameters: 1000 permutations gene set permutation type, using “weighted” enrichment statistic, and Signal2Noise as a metric for ranking genes. The leading-edge genes in each CD4+ T-cell gene set were compared to identify overlapping and unique genes. A spider plot depicting normalized enrichment scores from the GSEA (FIG. 14A) and a bar plot depicting the overlaps of the various gene sets with 4PD1^{hi} data set (FIG. 14B) are shown. EXH, exhausted CD4⁺ T cells; Tr⁻H, follicular helper T cells; nTREG, natural regulatory T cells (T_{regs}); iTREG, inducible T_{regs}; T_{H}1, T helper 1; T_{H}2, T helper 2; T_{H}17, T helper 17; EFF, effector CD4⁺ T cells; MEM, memory CD4⁺ T cells; Tr1, type 1 T_{regs}. FIG. 14C shows analysis of known T_{FH} differentially expressed genes (Choi et al., 2015; Kenefeck et al., 2015; Liu et al., 2012; Miyauchi et al., 2016) in 4PD1^{hi} and T_{reg} datasets (FIG. 12) in comparison with publicly available “bona fide” T_{FH} gene expression data (Miyauchi et al., 2016). Transcriptomes were normalized relative to the naïve T-cell dataset in each study to allow for a direct comparison. FIG. 14D shows mRNA expression of the indicated T_{FH}-associated genes by qPCR in splenic (upper graphs, SP) and tumor-derived (lower graphs, TM) 4PD1^{neg}, 4PD1^{hi} cells, and T_{regs} isolated from B16-bearing Foxp3-GFP transgenic mice (mean±SD of triplicates). Splenic T-cell subsets are compared with CXCR5⁺PD-1^{hi}Foxp3⁻CD4⁺ T_{FH} FACS-sorted from the spleen of Foxp3-GFP transgenic mice immunized with sRBC (average±SD of 3 biological replicates). FIG. 14E shows expression analyses by FACS of the indicated T_{FH}-associated markers in 4PD1^{neg}, 4PD1^{hi} cells and T_{regs} from tumors (TM) and spleens of naïve or B16 tumor-bearing (TB) mice. FIG. 14F shows CXCR5 and Bcl6 expression by FACS in 4PD1^{neg}, 4PD1^{hi} cells, and T_{regs} from B16-bearing mice treated with anti-CTLA-4 or control isotype IgG (100 μg). Data are the mean±SEM of 5 mice/group; unpaired t test: *p<0.05, **p<0.01, ***p<0.001, ****p<0.0001.

FIG. 15A-15C show T_{FH}-like phenotype in donor- and patient-derived 4PD1^{hi} cells. FIG. 15A shows expression analyses by FACS of the indicated T_{FH}, T_{reg} and memory T-cell markers in donor-derived circulating 4PD1^{neg}, 4PD1^{hi} cells, and T_{regs} (mean±SEM of 3-6 healthy donors depending on the marker). T_{regs} and 4PD1^{hi} were gated as live single CD45⁺CD4⁺Foxp3-positive (T_{regs}) and CD45⁺CD4⁺Foxp3-negativePD-1^{hi} (4PD1^{hi}), or live single CD45⁺CD4⁺CD25-positive (T_{regs}) and CD45⁺CD4⁺CD25-negativePD-1^{hi} (4PD1^{hi}) to measure CD25 and Foxp3 expression respectively. FIG. 15B shows the frequency of CXCR5⁺ and CD45RA⁺ cells, and CD25 MFI in circulating 4PD1^{neg}, 4PD1^{hi} cells, and T_{regs} from advanced melanoma patients before and during ipilimumab treatment (3 mg/kg, q3 wks; mean±SEM of 15-20 patients/time point). FIG. 15C shows CXCR5, BCL6, and CD25 MFI and CD45RA⁺ % in the indicated subsets gated on live single CD4⁺CD45⁺ cells from immunotherapy-naïve human melanoma lesions (left panels). Frequency of 4PD1^{neg}, 4PD1^{hi} cells and T_{regs} within the CD4⁺CXCR5⁺BCL6⁺ T_{FH} gate in the same samples and FACS plots depicting the gating strategy for this analysis are shown (right panels) (mean±SEM of 10 tumors). Paired t test: *p<0.05, **p<0.01, ***p<0.001, ****p<0.0001.

FIG. 16A-16C show the relationship between 4PD1^{hi} and the T_{FH} lineage. FIG. 16A shows unsupervised hierarchical clustering with the related heatmap of T_{H}17-associated genes (Kenefeck et al., 2015) in gene expression datasets from mouse splenic 4PD1^{neg}, 4PD1^{hi}, and T_{regs} (FIG. 12) functionally validated in 3 independent experiments (FIG. 3B, FIG. 4A-4B). FIG. 16B shows representative immunofluorescent staining of CD4 (AlexaFluor488), Foxp3 (AlexaFluor568), and PD-1 (AlexaFluor647) in tumor tissue sections from B16-bearing WT and Batf KO mice treated with αCTLA-4 (100 μg) or isotype-matched control IgG (scale bar=50 μm; 40X original magnification; inset, 60× original magnification) as quantified in FIG. 13C. Arrows indicate 4PD1^{hi} in tumors from WT mice. FIG. 16C shows CD86 expression in CD45.1⁺CD19⁺ B cells (top) and proliferation (CTV^{low}) of target naïve CD4⁺ T cells (bottom) co-cultured with or without T_{regs} in the presence of αCTLA-1 4 or control isotype IgG (mean±SD of triplicates cultures, unpaired t test). Representative plots from co-cultures treated with αCTLA-4 or control isotype IgG are shown. *p<0.05, **p<0.01, ***p<0.001, ****p<0.0001.

FIG. 17A-17E show dual opposing immune functions of 4PD1^{hi} cells. In FIG. 17A, B16-bearing Foxp3-GFP mice were immunized with sRBC as indicated, or left untreated (NT), and 4PD1^{hi} cells, total T_{regs}, and 4PD1^{neg} were FACS-sorted from tumors (left panels) or spleens (right panels) and tested in in vitro suppression assays with naïve CTV-labeled CD45.1⁺CD4⁺ target T cells. Proliferation (CTV^{low}) and activation (CD25⁺CD44⁺) of target cells co-cultured at 1:1 ratio with tumor-derived CD4⁺ T-cell subsets (left panel; mean±SD of 2-3 replicate cultures/condition, unpaired t test), or at different ratios with spleen-derived CD4⁺ T-cell subsets (right panels; mean±SD of 2-3 replicate cultures/condition, 2-way ANOVA), and Foxp3 and PD-1 expression in CD45.1⁻ 4PD1^{hi} cells, 4PD1^{neg}, or T_{regs} from the same co-cultures are reported. FIG. 17B shows B-cell activation assays with 4PD1^{neg}, 4PD1^{hi} cells, and total T_{regs}, FACS-sorted from spleens or tumors of untreated B16-bearing Foxp3-GFP mice. Representative FACS plots and quantification of CD86 (average±SEM of 2 or 3 independent experiments performed with tumor- or spleen-derived T cells respectively, unpaired t test) and MHC-II expression (I-A/I-E, average±SD of 3-5 replicate cultures/condition from one representative experiment, unpaired t test) on CD19⁺CD4⁻CD45.1⁺ target B cells stimulated alone or with the indicated CD4⁺ T-cell subsets (2:1 ratio) are shown. In FIG. 17C, naïve and B16-bearing mice were immunized with sRBC, CXCR5-positive and CXCR5-negative 4PD1^{hi} cells were sorted from spleens and tumors, along with 4PD1^{neg} and total T_{regs}, and were tested in B-cell activation (FIG. 17D) and T-cell suppression assays (FIG. 17E). FIG. 17D shows CD86 and MHC-II (I-A/I-E) expression in target CD45.1⁺CD4⁻CD19⁺ B cells stimulated in culture with the indicated CD4⁺ T-cell subsets at 2:1 ratio (mean±SD of 4-6 replicate cultures/condition, unpaired t test). FIG. 17E shows proliferation (CTV^{low}) of target CD45.1⁺CD4⁺ T cells co-cultured with the indicated CD4⁺ T-cell subsets at 1:1 ratio, and quantification of IL-2 in culture supernatants (0.4×10⁵ cells from spleen, SP; 0.1×10⁵ cells from tumor, TM; mean±SD of 2-4 replicate cultures; unpaired t test). *p<0.05, **p<0.01, ***p<0.001, ****p<0.0001.

FIG. 18A-18C show phenotypic and functional modulation of 4PD1^{hi} cells by sRBC immunization. FIG. 18A shows representative FACS plots showing modulation of 4PD1^{hi} % and CXCR5, Bcl6, and T-bet expression in 4PD1^{hi} cells from naïve and B16-bearing mice one week after immunization with sRBC in comparison with untreated mice (NT). 4PD1^{hi}, 4PD1^{neg} and T_{regs} were FACS-sorted from spleens (FIG. 18B) or tumors (FIG. 18C) of non-treated (NT) or sRBC-immunized B16-bearing Foxp3-GFP transgenic mice as shown in FIG. 7A and tested in suppression assays. FIG. 18B shows proliferation of CD45.1⁺CD8⁺ target T cells (CTV^{low}) cultured with the indicated spleen-derived CD4⁺ T-cell subsets and quantification of IFN-γ and TNF-α in culture supernatants after 48-hour incubation (mean±SD of 3 replicate cultures). FIG. 18C shows proliferation (CTV^{low}) and activation (CD25⁺CD44⁺) of CD45.1⁺CD4⁺ target T cells co-cultured at 1:1 ratio with the indicated tumor-derived CD4⁺ T-cell subsets (mean±SD of 2-3 replicate cultures/condition). Unpaired t test: *p<0.05, **p<0.01, ***p<0.001.

FIG. 19 show a T-cell dependent B-cell activation assay. Culture stimulation conditions used in B-cell activation assays shown in FIGS. 17B and 17D for the detection of T-cell-mediated effects on B cells. CD19⁺CD4⁻CD45.1⁺ B cells were cultured alone (B cells alone) or with CD45.1⁻CD4⁺ T cells (B cells+T_{eff}) and stimulated (STIM) or not (NS) with PHA+IL-2. After a 48-hr incubation, B-cell expression of CD86 and MHC-II (I-A/I-E) were quantified by FACS. In these conditions, activation of B cells is observed only when they are stimulated in the presence of T cells (T-cell dependent B-cell activation). Data are the mean±SD of triplicate cultures. Unpaired t test: **p<0.01, ***p<0.001.

FIG. 20A-20C show a functional comparison of 4PD1^{hi} cells, T_{regs}, and Tₘₑₘ in suppression assays. 4PD1^{hi}, memory CD4⁺ T cells (CD44⁺PD-1⁻Foxp3⁻CD4⁺ T cells; Tₘₑₘ), and T_{regs} (Foxp3⁺CD4⁺ T cells) were sorted from the spleens of Foxp3-GFP transgenic mice immunized with sRBC (FIG. 20A), or tumor-bearing mice treated with four courses of anti-CTLA-4 (FIG. 20B). These three cell subsets were tested individually in standard suppression assays with activated CellTraceViolet (CTV)-labeled CD8⁺ (top panels) or CD4⁺ (bottom panels) target T cells from naïve CD45.1⁺ congenic mice at the indicated effector:target ratios. Proliferation (CTV^{low} %) and activation (CD25⁺CD44⁺ %) of target CTV⁺CD45.1⁺CD8⁺ and CD4⁺ T cells were quantified in each condition. FIG. 20C shows results of suppression assays with 4PD1^{hi}, Tₘₑₘ, and T_{regs} FACS-sorted from the tumors of anti-CTLA-4 treated Foxp3-GFP transgenic mice. These 3 cell subsets were tested individually with target CD8⁺ or CD4⁺ T cells a 1:1 effector:target ratio. Representative plots show the gating strategy used to sort 4PD1^{hi} cells, Tₘₑₘ, and T_{regs} from the different tissues and baseline CD44 expression in the three sorted cell subsets. These results confirm the lack of functional and phenotypic overlap between 4PD1^{hi} and conventional memory T cells.

FIG. 21A-21B show expression of immunosuppressive genes in 4PD1^{hi}. Unsupervised hierarchical clustering with the related heatmaps of immune inhibitory genes (Table 4) in RNAseq data sets from mouse splenic (FIG. 21A) and donor-derived (FIG. 21B) 4PD1^{neg} and T_{regs} (FIG. 12). Genes overexpressed in 4PD1^{hi} are highlighted with a black line.

### DETAILED DESCRIPTION OF THE INVENTION

We demonstrate that 4PD1^{hi} cells are present at low frequency in the circulation of normal hosts, accumulate at the tumor site as a function of tumor burden, and constitutively inhibit T-cell functions in a PD-1/PD-L1 dependent fashion. CTLA-4 blockade promotes intratumoral and peripheral increases in 4PD1^{hi} cells in a dose-dependent manner, while combination with PD-1 blockade mitigates this effect and significantly improves anti-tumor activity. Patients have a significantly higher risk of death if high 4PD1^{hi} cell levels persist after PD-1 blockade. Accordingly, we provide a new pharmacodynamic and prognostic biomarker that can improve treatment of cancer by informing the design of optimal combination schedules and checkpoint blockade dosage.

The observation that 4PD1^{hi} cells increase and accumulate within the tumor microenvironment as a function of tumor growth indicates that persistent tumor-antigen exposure may facilitate and sustain their generation. Given that chronic antigen stimulation is a prerequisite for both conventional T_{FH} development (Baumjohann et al., 2013) and T-cell exhaustion (Wherry and Kurachi, 2015), these two outcomes may result from common molecular pathways. This is in line with recent studies in chronic infection models reporting induction of a T_{FH}-like CXCR5⁺CD8⁺ T-cell pool with a partially exhausted phenotype, which is reversible with PD-1 pathway blockade (He et al., 2016; Im et al., 2016). In the CD4⁺ T-cell compartment, this process may lead to the acquisition of a T-cell inhibitory capacity. Our results indicate that tumor-induced 4PD1^{hi} cells, while sustaining B-cell stimulation, affect T-cell effector function in a way that is also reversible with PD-1 pathway blockade.

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10. Where a numeric term is preceded by “about,” the term includes the stated number and values±10% of the stated number. The headings provided herein are not limitations of the various aspects or embodiments of the invention, which can be had by reference to the specification as a whole. Accordingly, the terms defined immediately below are more fully defined by reference to the specification in its entirety.

Amino acids are referred to herein by their commonly known three-letter symbols or by the one-letter symbols recommended by the IUPAC-IUB Biochemical Nomenclature Commission. Nucleotides, likewise, are referred to by their commonly accepted single-letter codes. Unless otherwise indicated, amino acid sequences are written left to right in amino to carboxy orientation, and nucleic acid sequences are written left to right in 5′ to 3′ orientation.

Wherever embodiments are described with the language “comprising,” otherwise analogous embodiments described in terms of “consisting of” and/or “consisting essentially of” are included.

The term “immune checkpoint blockade” or “ICB,” as used herein, refers to the administration of one or more inhibitors of one or more immune checkpoint proteins or their ligand(s). Immune checkpoint proteins include, but are not limited to, cytotoxic T lymphocyte-associated antigen 4 (CTLA-4), also known as CD152, programmed cell death protein 1 (PD-1), also known as CD279, lymphocyte-activation gene 3 (LAG-3), also known as CD223, and T cell immunoglobulin mucin (TIM-3), also known as HAVcr2.

An “active agent” is an agent which itself has biological activity, or which is a precursor or prodrug that is converted in the body to an agent having biological activity. Active agents useful in the methods of the invention include inhibitors of immune checkpoint proteins or their ligand(s), for example, CTLA-4 inhibitors (including antibodies to CTLA-4 that inhibit its function), PD-1 inhibitors (including antibodies to PD-1 that inhibit its function), and PD-L1 inhibitors (including antibodies to PD-1 ligand that inhibit its function).

The terms “inhibit,” “block,” and “suppress” are used interchangeably and refer to any statistically significant decrease in biological activity, including full blocking of the activity. An “inhibitor” is an active agent that inhibits, blocks, or suppresses biological activity in vitro or in vivo. Inhibitors include but are not limited to small molecule compounds; nucleic acids, such as siRNA and shRNA; polypeptides, such as antibodies or antigen-binding fragments thereof, dominant-negative polypeptides, and inhibitory peptides; and oligonucleotide or peptide aptamers.

A “CTLA-4 inhibitor” is an active agent that antagonizes the activity of cytotoxic T lymphocyte-associated antigen 4 or reduces its production in a cell. Examples of CTLA-4 inhibitors that are suitable for use in the present invention include ipilimumab and tremelimumab. Derivatives of these compounds that act as CTLA-4 inhibitors are also suitable for use in the invention.

A “PD-1 inhibitor” is an active agent that antagonizes the activity of programmed cell death protein 1 or reduces its production in a cell. Examples of PD-1 inhibitors that are suitable for use in the present invention include nivolumab, pembrolizumab, pidilizumab, and REGN2810. PD-1 inhibitors also include active agents that inhibit the PD-1 ligand (PD-L1), including atezolizumab, avelumab, durvalumab, and BMS-936559. Derivatives of the foregoing compounds that act as PD-1 inhibitors are also suitable for use in the invention.

As used herein, the term “gene expression signature” is used consistently with its conventional meaning in the art, and refers to an expression profile of a group of genes that is characteristic of a certain cell type, a certain cell population, a certain biological phenotype, or a certain medical condition. By way of example, when the term “gene expression signature” is used in relation to 4PD1hi cells, it refers to an expression profile of a group of genes that is characteristic of 4PD1hi cells. For example, and as described below, 4PD1hi cells are CD4-positive, Foxp3-negative, and PD-1-positive—i.e. 4PD1hi cells can be characterized by the “gene expression signature” CD4⁺Foxp3⁻PD-1⁺. Gene expression signatures can be determined using any suitable method known in the art for determining the expression of a gene, including, but not limited to, those that detect and/or measure gene expression at the mRNA level or the protein level, such as RT-PCR-based methods, immunohistochemistry (IHC)-based methods, flow cytometry-based methods, and the like.

“4PD1^{hi}” cells are a subset of CD4⁺Foxp3⁻ T cells expressing PD-1. 4PD1^{hi} cell frequency is measured as a percentage of CD4+ cells. Cell frequency can be measured or quantified by any method known in the art. Examples of suitable techniques include, but are not limited to, those that involve immunohistochemistry (IHC), flow cytometry, and/or PCR, each of which technique can be used to detect, measure, and/or quantify cells having a given gene expression signature. 4PD1^{hi} cell frequency can be measured according to the methods of the invention at least about one, two, three, four, five, or six weeks after a dose of ICB therapy. In some cases, 4PD1^{hi} cell frequency is measured before the dose of ICB therapy to determine a patient's baseline 4PD1^{hi} cell frequency. Because ICB therapy is typically cyclical (for example, one dose is administered every three weeks for a total of four doses), a baseline 4PD1^{hi} cell frequency can be acquired before the first dose or before one or more subsequent doses.

A 4PD1^{hi} cell frequency of 2.2% or greater is “high,” while a 4PD1^{hi} cell frequency of less than 2.2% is “low.” Patients having a high 4PD1^{hi} cell frequency can be classified as resistant to ICB therapy, and can be treated with a higher dosage of PD-1 inhibitor and/or a lower (including no) dosage of CTLA-4 inhibitor, relative to, for example, either a prior dose received by the patient or a standard dose. Conversely, patients having a low 4PD1^{hi} cell frequency can be classified as susceptible to ICB therapy, and can be treated with a lower (including no) dosage of PD-1 inhibitor and/or a higher dosage of CTLA-4 inhibitor, relative to either a prior dose received by the patient or the standard dose.

A “standard dose” of ICB therapy is known by a person of skill in the art for each medication, and may be the dose that is indicated in the prescribing information and/or the dose that is most frequently administered under particular clinical circumstances (for example for the particular PD-1 inhibitor and/or CTLA-4 inhibitor being used, the particular route of administration being used, the particular cancer being treated, the age, weight, and/or sex of the particular patient, etc.). In some embodiments, a standard dose of ICB therapy is about 1-3 mg/kg. In some embodiments, a standard dose of ICB therapy is about 1 mg/kg. In some embodiments, a standard dose of ICB therapy is about 2 mg/kg. In some embodiments, a standard dose of ICB therapy is about 3 mg/kg.

Patients having a 51% or less reduction (≤0.49-fold change) in 4PD1^{hi} cells after a dose of ICB therapy, as compared to a baseline level of 4PD1^{hi} cells, can be classified as resistant to ICB therapy. Such patients can be treated with a higher dosage of PD-1 inhibitor and/or a lower (including no) dosage of CTLA-4 inhibitor, relative to the prior dose received by the patient. Patients having a greater than 51% reduction (>0.49-fold change) in 4PD1^{hi} cells after a dose of ICB therapy, as compared to a baseline level of 4PD1^{hi} cells, can be classified as susceptible to ICB therapy. Such patients can be treated with a lower (including no) dosage of PD-1 inhibitor and/or a higher dosage of CTLA-4 inhibitor, relative to the prior dose received by the patient.

For example, in some embodiments the methods of the present invention involve measuring 4PD1^{hi} cell frequency in a blood sample from a patient after the patient has received a first dose of ICB therapy using a first dosage of a PD-1 inhibitor and/or a CTLA-4 inhibitor, and subsequently administering a second dose of ICB therapy to the patient using a second dosage of the PD-1 inhibitor and/or the CTLA-4 inhibitor, wherein an adjustment from the first dosage to the second dosage is made based on the patient's 4PD1^{hi} cell frequency. For example, in some embodiments, the second dosage of a PD-1 inhibitor is increased as compared to the first dosage of the PD-1 inhibitor if the 4PD1^{hi} cell frequency is high. In some embodiments, the second dosage of a PD-1 inhibitor is decreased as compared to the first dosage of the PD-1 inhibitor if the 4PD1^{hi} cell frequency is low. In some embodiments, the second dosage of a CTLA-4 inhibitor is increased as compared to the first dosage of the CTLA-4 inhibitor if the 4PD1^{hi} cell frequency is low. In some embodiments, the second dosage of a CTLA-4 inhibitor is decreased as compared to the first dosage of the CTLA-4 inhibitor if the 4PD1^{hi} cell frequency is high. Typically, the first dosage of the PD-1 and/or CTLA-4 inhibitor in such embodiments is either a dose that has previously been used to treat the same patient, or a standard dose. In those embodiments where the second dosage of the PD-1 inhibitor or CTLA-4 inhibitor is increased as compared to the first dosage, the dosage may be increased by about 10%, or about 20%, or about 30%, or about 40%, or about 50%, or about 60%, or about 70%, or about 80%, or about 90%, or about 100%, or about 125%, or about 150%, or about 175%, or about 200%, or about 300%, or about 400%, or about 500%, or more. Conversely, in those embodiments where the second dosage of the PD-1 inhibitor or CTLA-4 inhibitor is decreased as compared to the first dosage, the dosage may be decreased by about 10%, or about 20%, or about 30%, or about 40%, or about 50%, or about 60%, or about 70%, or about 80%, or about 90%, or more, up to 100%.

By way of a further example, in some embodiments the methods of the present invention involve predicting a patient's response to ICB therapy based on the frequency of 4PD1^{hi} cells the patient's blood, classifying the patient as susceptible to ICB therapy if the 4PD1^{hi} cell frequency is low, or resistant to ICB therapy if the 4PD1^{hi} cell frequency is high (as described above), and administering a lower dosage of a PD-1 inhibitor and/or a higher dosage of a CTLA-4 inhibitor if the patient is susceptible to ICB therapy, or a higher dosage of a PD-1 inhibitor and/or a lower dosage of a CTLA-4 inhibitor wherein the patient is resistant to ICB therapy. A “lower dosage” is a dosage of that is lower (for example about 10%, or about 20%, or about 30%, or about 40%, or about 50%, or about 60%, or about 70%, or about 80%, or about 90%, or more, up to 100% lower) than either a dose that has previously been used to treat the same patient, or a standard dose. Conversely a “higher dosage” is a dosage of that is higher (for example about 10%, or about 20%, or about 30%, or about 40%, or about 50%, or about 60%, or about 70%, or about 80%, or about 90%, or about 100%, or about 125%, or about 150%, or about 175%, or about 200%, or about 300%, or about 400%, or about 500%, or more, higher) than either a dose that has previously been used to treat the same patient, or a standard dose.

By “subject” or “individual” or “patient” is meant any subject, preferably a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, and so on.

Patients to whom the methods and uses of the invention can be applied may be undergoing ICB therapy for any type of cancer. Examples include melanoma, skin carcinoma, non-small cell lung cancer (NSCLC), kidney cancer, bladder cancer, head and neck cancers, lymphoma, breast cancer, ovarian cancer, prostate cancer, pancreatic cancer, colorectal cancer, gastric cancer, and esophageal cancer.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder according to the methods provided herein if the patient shows, e.g., total, partial, or transient alleviation or elimination of symptoms associated with the disease or disorder.

“Prevent” or “prevention” refers to prophylactic or preventative measures that prevent and/or slow the development of a targeted pathologic condition or disorder. Thus, those in need of prevention include those at risk of or susceptible to developing the disorder. In certain embodiments, a disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

The term “pharmaceutical composition” refers to a preparation that is in such form as to permit the biological activity of the active ingredient to be effective, and which contains no additional components that are unacceptably toxic to a subject to which the composition would be administered. Pharmaceutical compositions can be administered in any of numerous dosage forms, for example, tablet, capsule, liquid, solution, softgel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. Pharmaceutical compositions typically comprise a pharmaceutically acceptable carrier, and can comprise one or more of a buffer (e.g. acetate, phosphate or citrate buffer), a surfactant (e.g. polysorbate), a stabilizing agent (e.g. human albumin), a preservative (e.g. benzyl alcohol), a penetration enhancer, an absorption promoter to enhance bioavailability and/or other conventional solubilizing or dispersing agents. Choice of dosage form and excipients depends upon the active agent to be delivered and the disease or disorder to be treated or prevented, and is routine to one of ordinary skill in the art.

“Systemic administration” means that a pharmaceutical composition is administered such that the active agent enters the circulatory system, for example, via enteral, parenteral, inhalational, or transdermal routes. Enteral routes of administration involve the gastrointestinal tract and include, without limitation, oral, sublingual, buccal, and rectal delivery. Parenteral routes of administration involve routes other than the gastrointestinal tract and include, without limitation, intravenous, intramuscular, intraperitoneal, intrathecal, and subcutaneous. “Local administration” means that a pharmaceutical composition is administered directly to where its action is desired (e.g., at or near the site of the injury or symptoms). Local routes of administration include, without limitation, topical, inhalational, subcutaneous, ophthalmic, and otic. It is within the purview of one of ordinary skill in the art to formulate pharmaceutical compositions that are suitable for their intended route of administration.

An “effective amount” of a composition as disclosed herein is an amount sufficient to carry out a specifically stated purpose. An “effective amount” can be determined empirically and in a routine manner, in relation to the stated purpose, route of administration, and dosage form.

In some embodiments, administration of ICB therapy can comprise systemic administration, at any suitable dose and/or according to any suitable dosing regimen, as determined by one of skill in the art. The immune checkpoint inhibitor(s) can be administered according to any suitable dosing regimen, for example, where the daily dose is divided into two or more separate doses. It is within the skill of the ordinary artisan to determine a dosing schedule and duration for administration.

Embodiments of the present disclosure can be further defined by reference to the following non-limiting examples. It will be apparent to those skilled in the art that many modifications, both to materials and methods, can be practiced without departing from the scope of the present disclosure.

### EXAMPLES

### Example 1. CD4+Foxp3− T Cells Expressing PD-1 (4PD1hi) Accumulate at the Tumor Site in Mice and Humans

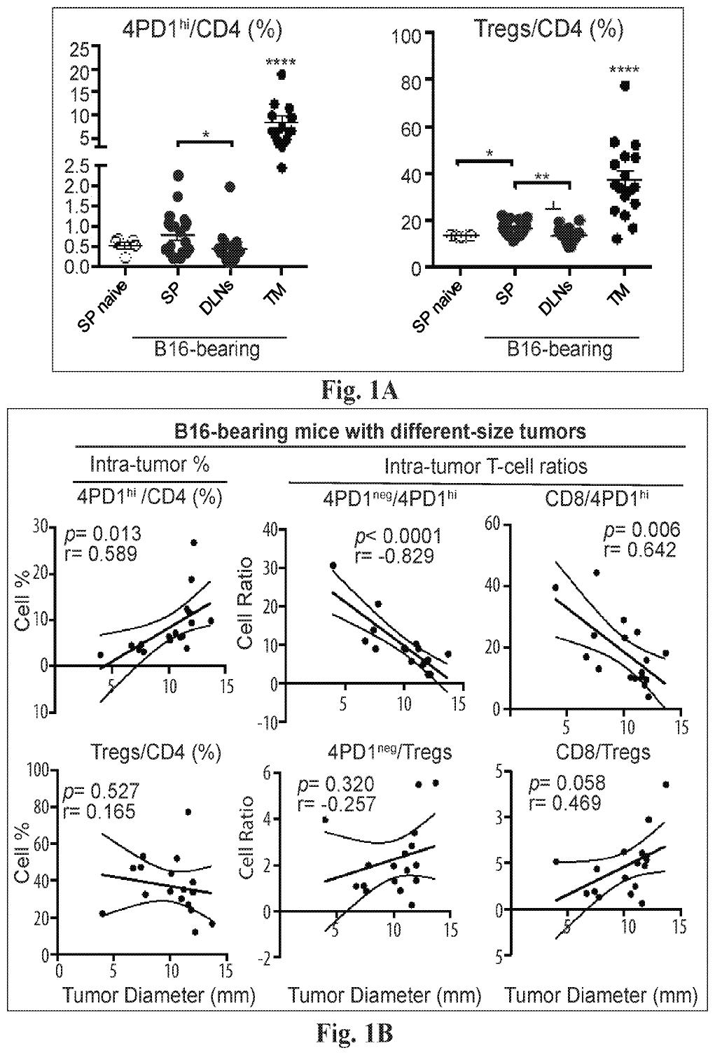

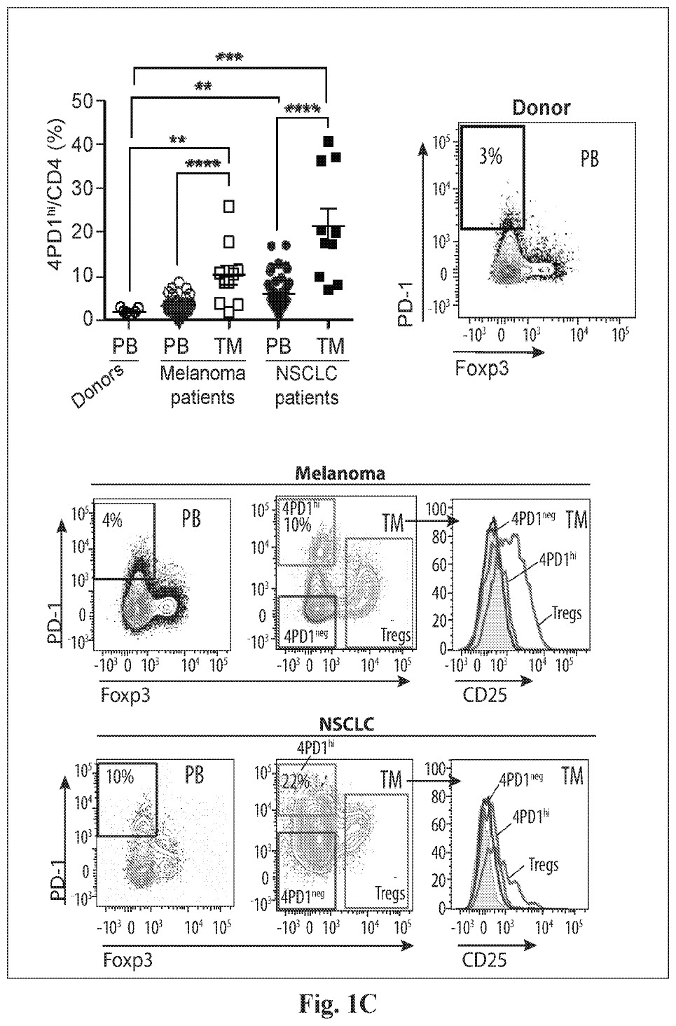

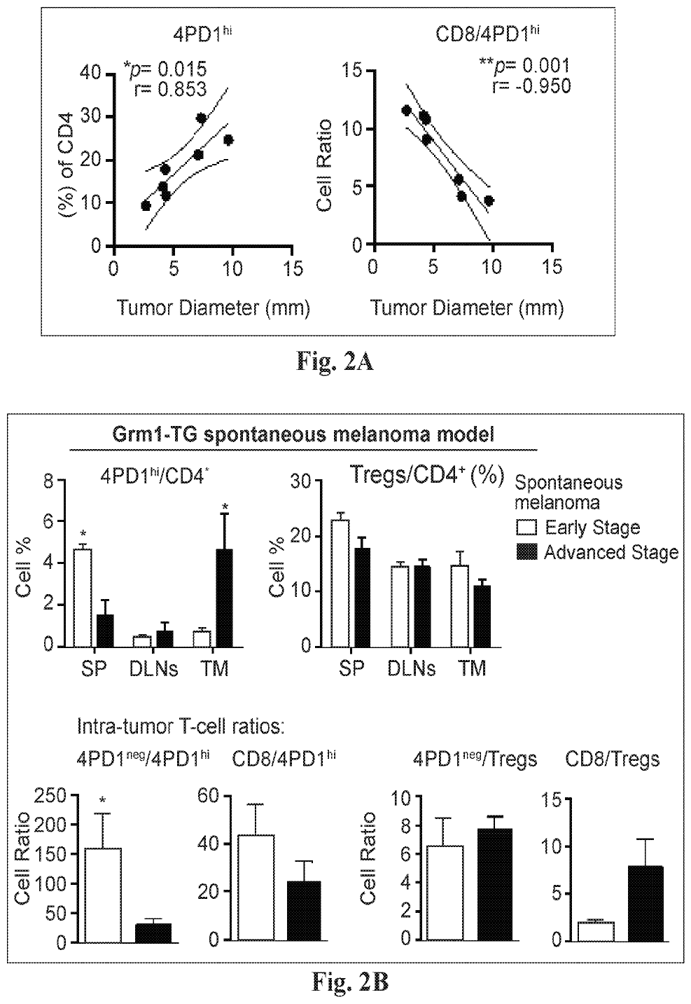

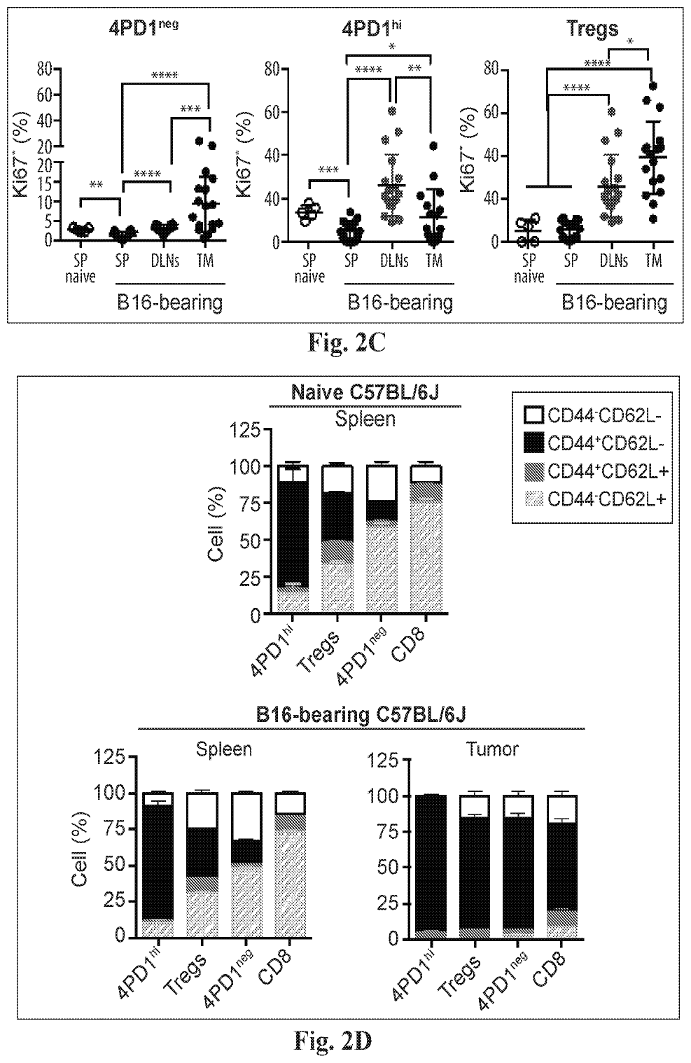

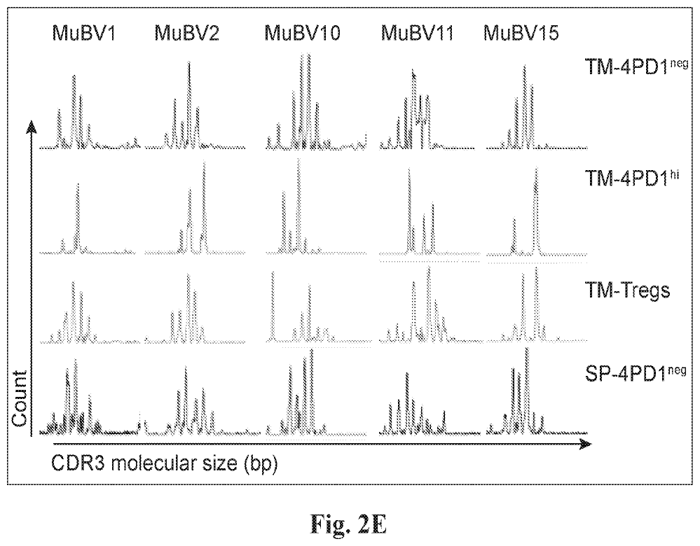

We assessed the tissue distribution of 4PD1^{hi} in untreated naïve and tumor-bearing mice (FIG. 1A). We observed that 4PD1^{hi}, similar to T_{regs}, are significantly enriched at the tumor site compared to secondary lymphoid organs in B16 melanoma-bearing mice (FIG. 1A). To test whether intra-tumor 4PD1^{hi} accumulation correlates with tumor burden, we analyzed 4PD1^{hi} frequency in correlation with tumor size in mice injected with increasing amounts of B16 cells (FIG. 1B). We found that intra-tumor 4PD1^{hi} accumulate as a function of tumor size and the ratios between Foxp3⁻PD-1⁻CD4⁺ (4PD1^{neg}) or CD8⁺ T_{eff} and 4PD1^{hi} inversely correlate with tumor burden (FIG. 1B). Of note, when the same analyses were performed with T_{regs}, correlations were not statistically significant (FIG. 1B). We confirmed these results in mice implanted with the same number of B16 cells (FIG. 2A) and further substantiated the association between intra-tumor 4PD1^{hi} accumulation and tumor progression in genetically engineered mice that develop melanoma spontaneously (Grm1-TG) (Pollock et al., 2003). The frequency of 4PD1^{hi} was significantly higher and T_{eff}/4PD1^{hi} ratios reduced in advanced- (6-month-old) compared to early-stage (3-month-old) tumors in these mice (FIG. 2B). Interestingly, peripheral 4PD1^{hi} increases preceded their intra-tumor accumulation, as splenic 4PD1^{hi} were significantly augmented in the presence of early- compared to advanced-stage tumors (FIG. 2B). Analyses of proliferation potential, maturation status and TCR repertoire diversity of 4PD1^{hi} in comparison with the other CD4⁺ T-cell subsets revealed that 4PD1^{hi} proliferate more actively in tumor-draining lymph nodes (FIG. 2C), display a similar effector memory phenotype independent of anatomic location (FIG. 2D) and have an oligoclonal TCR repertoire, especially at the tumor site (FIG. 2E).

To test relevance of this cell population in cancer patients, we took advantage of our access to melanoma and NSCLC samples from immunotherapy-naïve patients in our tissue bank and quantified 4PD1^{hi} frequency in the periphery and at the tumor site. We observed that 4PD1^{hi} frequency is significantly higher in tumor compared to PB in melanoma and NSCLC patients (FIG. 1C), indicating that these cells accumulate intratumorally in humans, as they do in mice. Importantly, 4PD1^{hi} lack both Foxp3 and CD25 expression, thus confirming the non-T_{reg} phenotype of this cell subset (FIG. 1C, right panels).

These results indicate that 4PD1^{hi} are a pool of mature, likely antigen-experienced, cells that exist in naïve and tumor-bearing hosts, and preferentially expand in the periphery and accumulate at the tumor site as a function of tumor burden in both human and mice.

### Example 2. Mouse and Human 4PD1hi Limit T-Cell Effector Functions

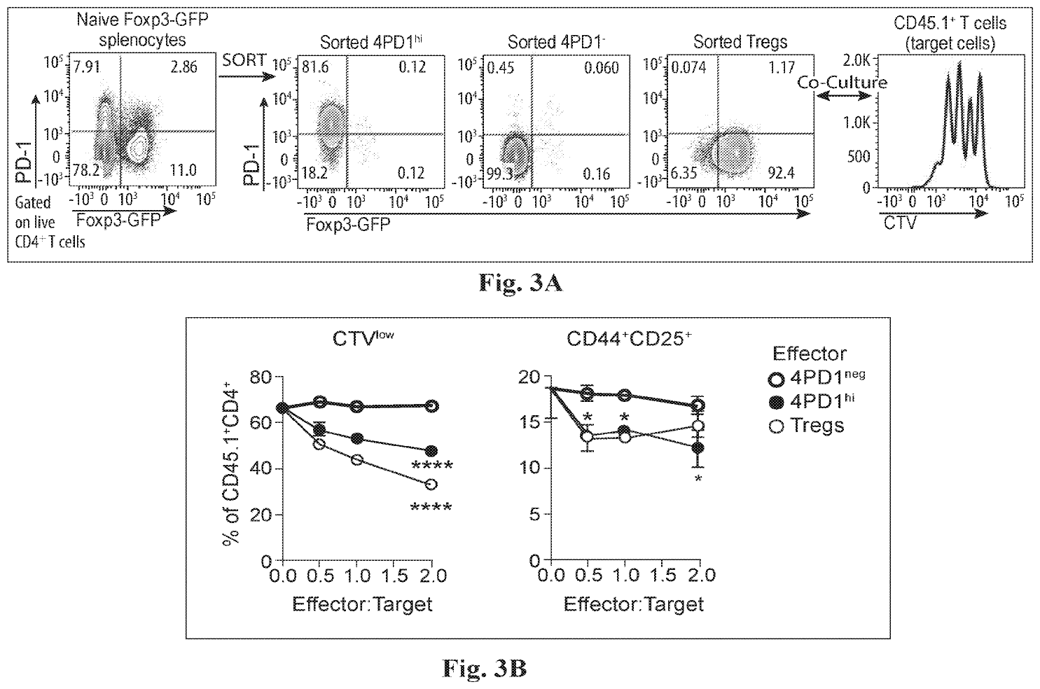

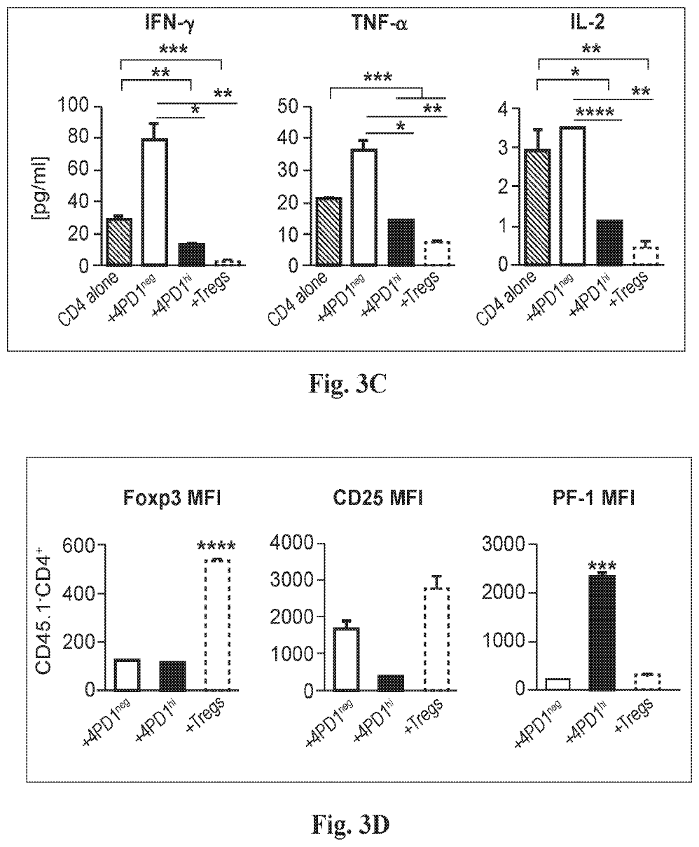

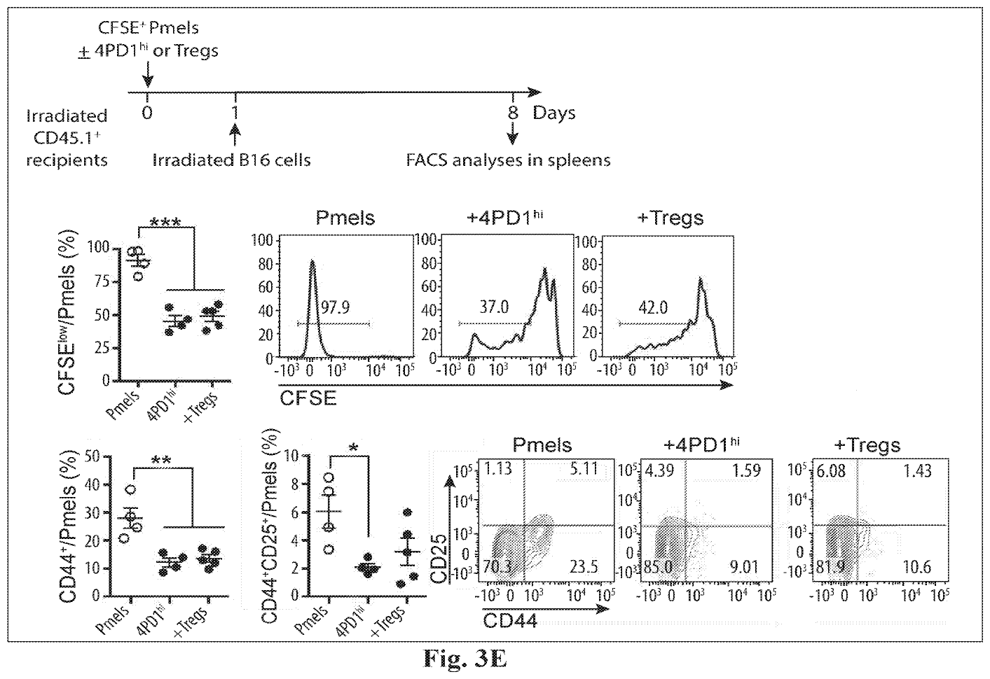

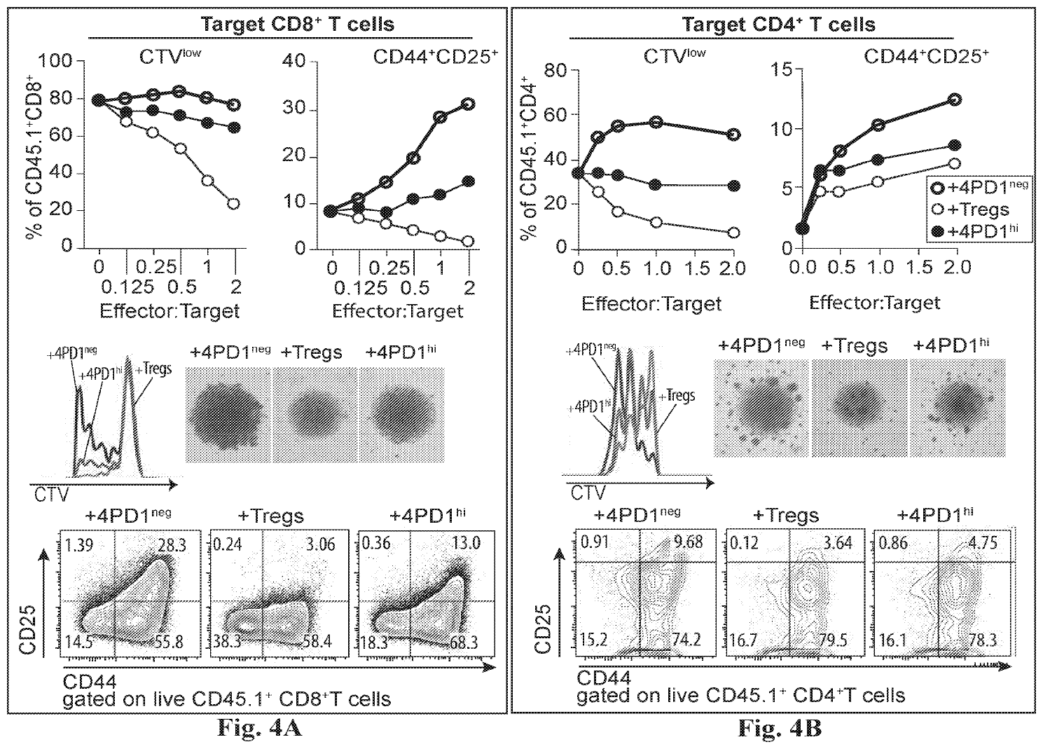

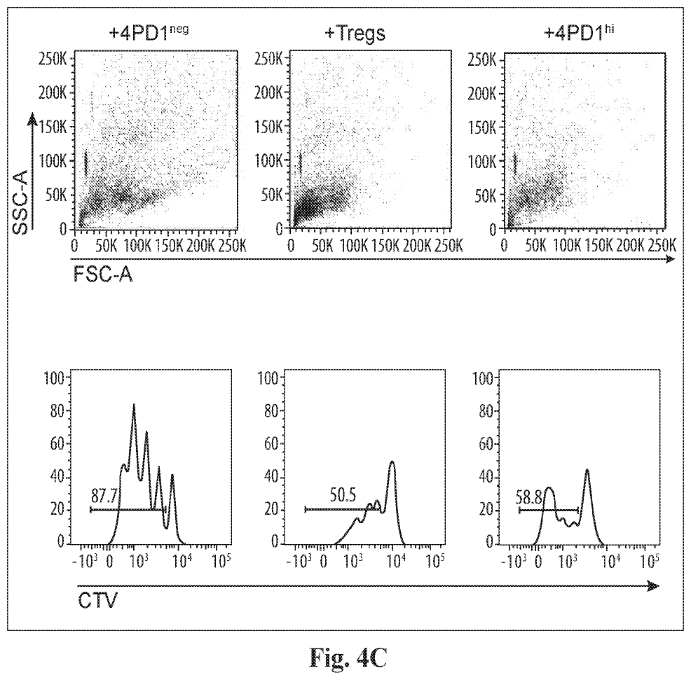

To determine whether 4PD1^{hi} could contribute to tumor immune escape mechanisms, we tested these cells in different types of in vitro and in vivo suppression assays. To isolate mouse Foxp3-negative PD-1^{hi} (4PD1^{hi}) and mouse Foxp3-negative PD-1-negative CD4⁺ T cells (4PD1^{neg}) as a control, we took advantage of Foxp3-GFP transgenic mice, where the transcription factor Foxp3 can be tracked by GFP expression. We first tested 4PD1^{hi} from spleens of naïve Foxp3-GFP mice in standard suppression assays (FIG. 3A). 4PD1^{neg} and PD-1-negative T_{regs} were used respectively as negative and positive controls for T-cell suppression (FIG. 3A). Naïve splenic 4PD1^{hi} significantly reduced proliferation and activation of polyclonal CD4⁺ or CD8⁺ T cells, although to a lesser extent than T_{regs} (FIG. 3B, FIG. 4A-4B). We excluded the possibility that these observations could be the consequence of competition for proliferation between target T cells and 4PD1^{hi} because 4PD1^{hi} were not capable of sustained division in culture (FIG. 4C). Consistently, IFN-γ, TNF-α and IL-2 were significantly reduced in cultures with target CD4⁺ or CD8⁺ T cells and either 4PD1^{hi} or T_{regs} with respect to 4PD1^{neg} (total CD4⁺ T cells, FIG. 3C; total CD8⁺ T cells). The inhibitory function of 4PD1^{hi} cannot be attributed to their acquisition of a T_{reg} phenotype in these assays, as highlighted by lack of Foxp3 or CD25 up-regulation (FIG. 3D). To verify these effects in vivo, we monitored proliferation and activation of Pmel-1/gp100-specific CD8⁺ T cells adoptively transferred in conjunction with 4PD1^{hi} or T_{regs} from tumor-bearing mice and stimulated with the injection of irradiated B16 as delineated in FIG. 3E. Co-transfer of 4PD1^{hi} or T_{regs} similarly and negatively affected proliferation and up-regulation of the activation markers CD44 and CD25 in Pmel-1/gp100-specific CD8⁺ T cells (FIG. 3E).

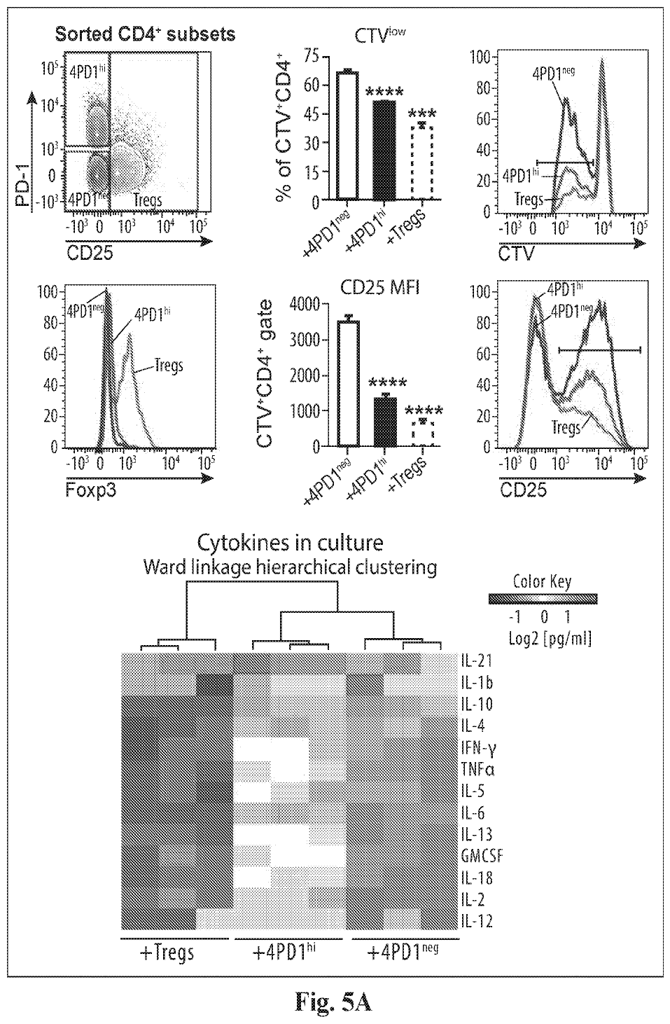

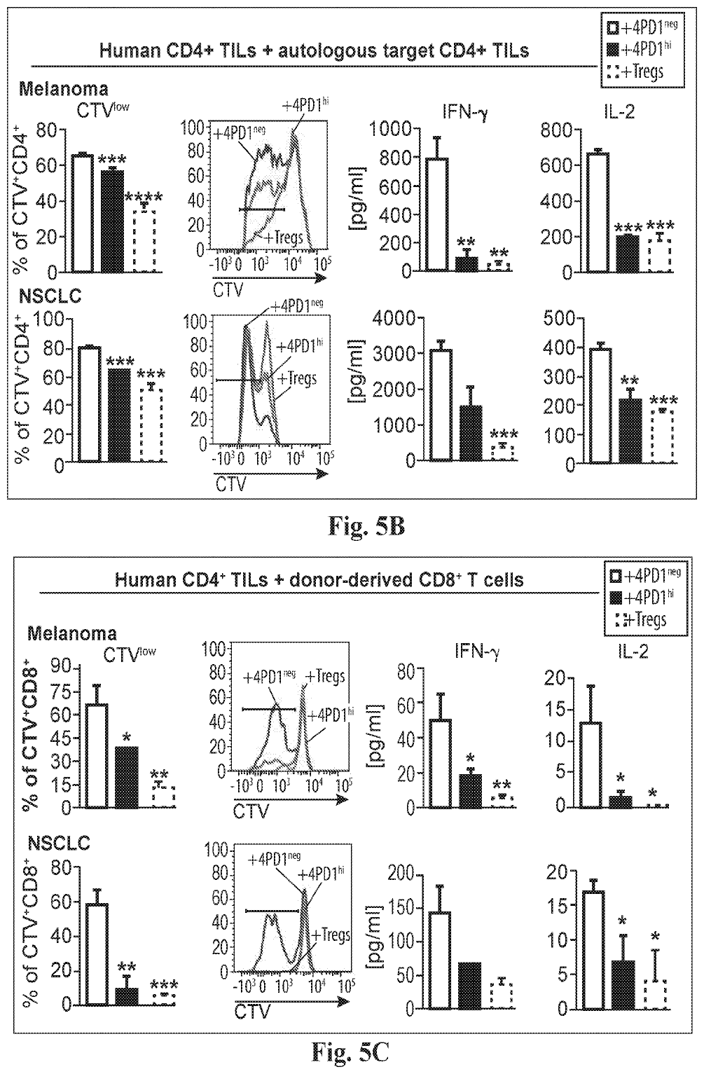

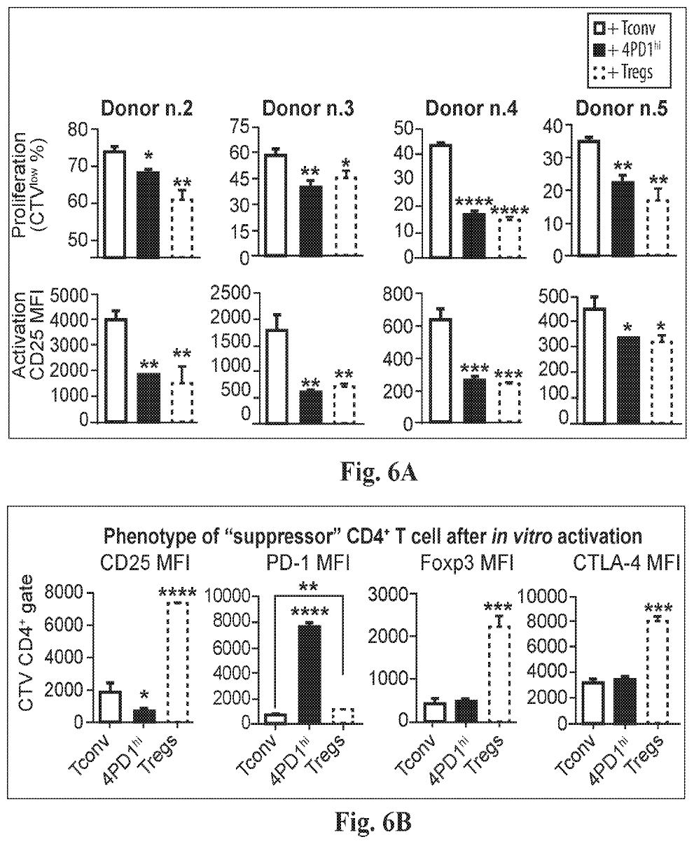

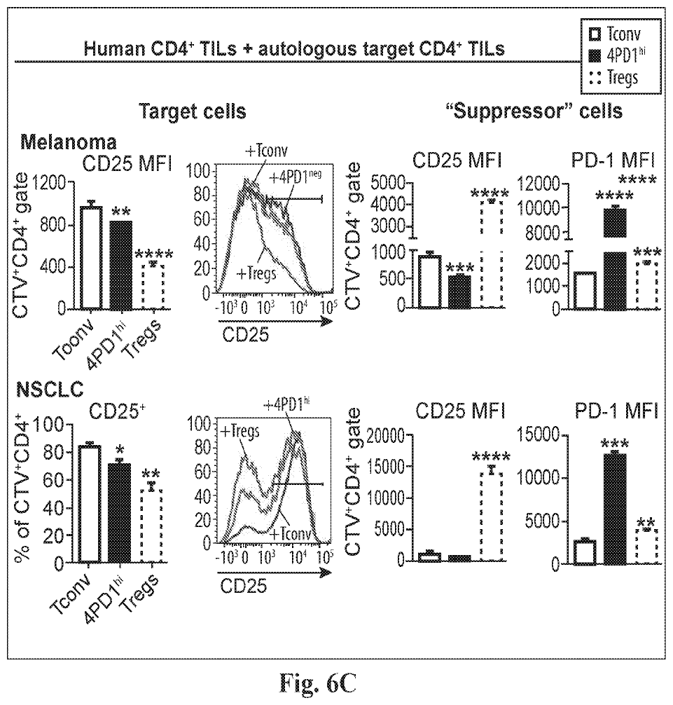

We thus asked whether human 4PD1^{hi} could limit T-cell function in a similar way and can promote tumor immune evasion. We took advantage of differential CD25 expression between 4PD1^{hi} and T_{regs} (FIG. 1C) to separate these two cell subsets from human samples and compared them in functional assays. Circulating donor-derived 4PD1^{hi} significantly reduced proliferation, activation, and production of pro-inflammatory cytokines of target T cells in comparison with 4PD1^{neg} (FIG. 5A, FIG. 6A), and did not acquire expression of T_{reg}-associated markers in culture (FIG. 6B). In concordance with results in mice, the inhibitory capacity of human 4PD1^{hi} was consistent yet not always as potent as that of T_{regs} (FIG. 5A, FIG. 6A). We next tested the function of 4PD1^{hi} from human tumors in similar conditions. 4PD1^{hi} from melanoma and NSCLC lesions consistently diminished proliferation, activation and production of pro-inflammatory cytokines of either autologous tumor-infiltrating (TILs) or donor-derived peripheral T cells (FIG. 5B-5C, FIG. 6C left panels) and maintained a distinct phenotype in culture (FIG. 6C right panels).

These results indicate that human and mouse 4PD1^{hi} are functional and have a constitutive capacity to limit T_{eff} functions, suggesting that they could be relevant in therapeutic settings.

### Example 3. 4PD1hi Modulation During Immune Checkpoint Blockade

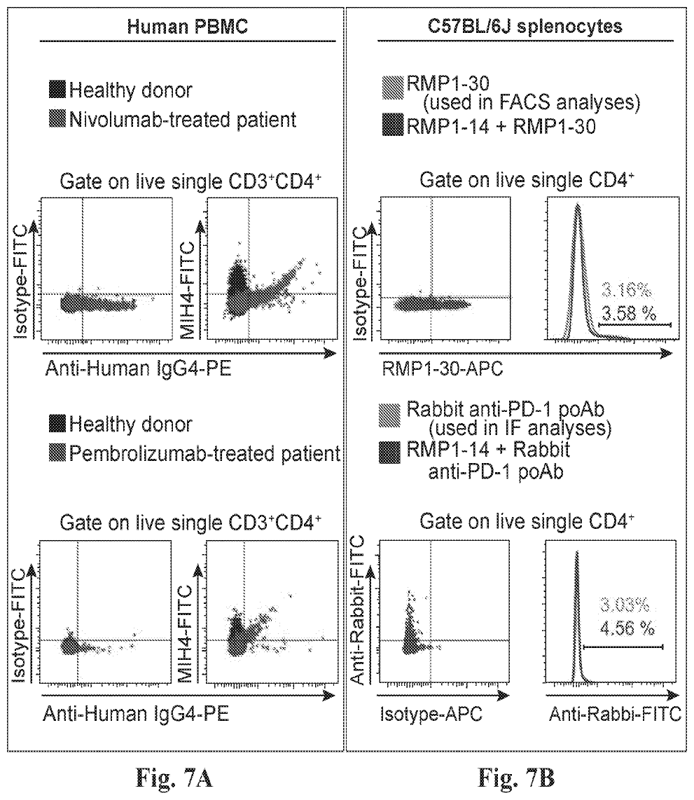

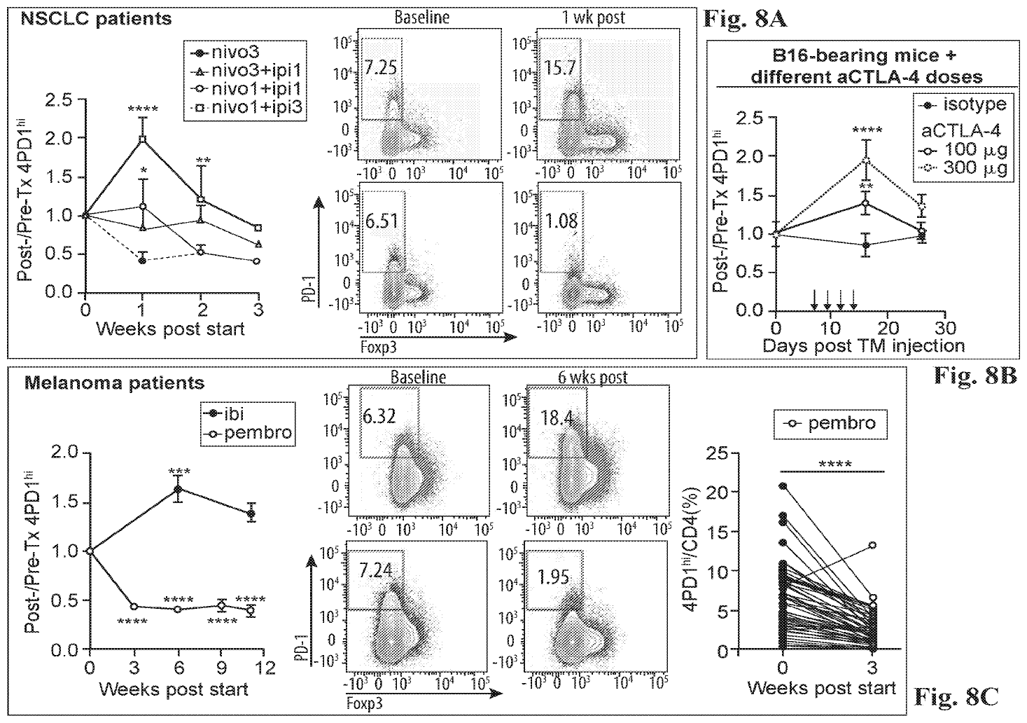

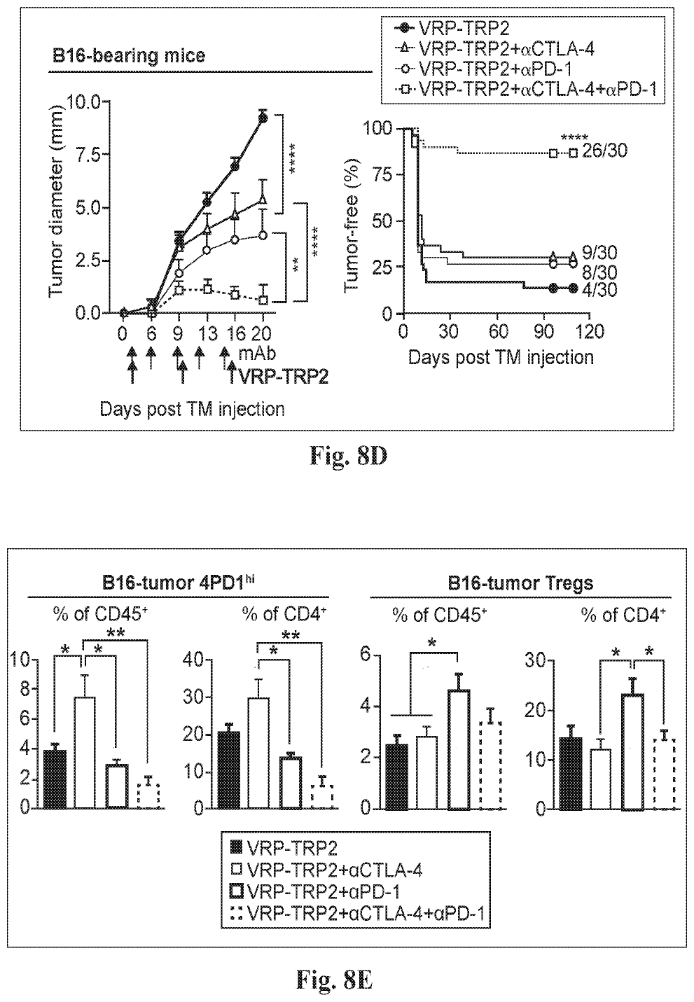

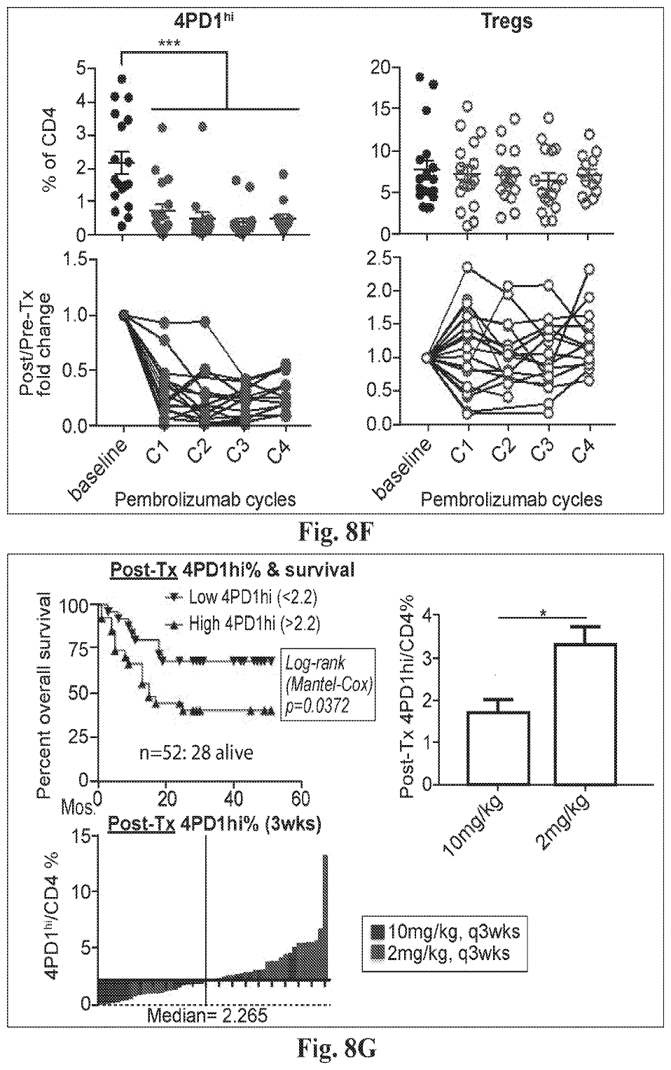

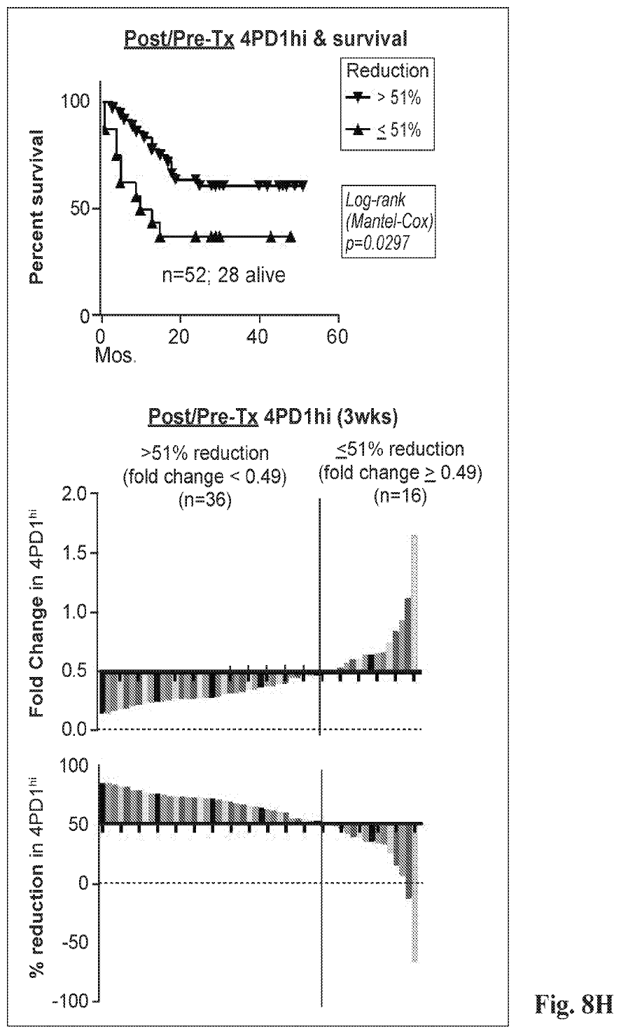

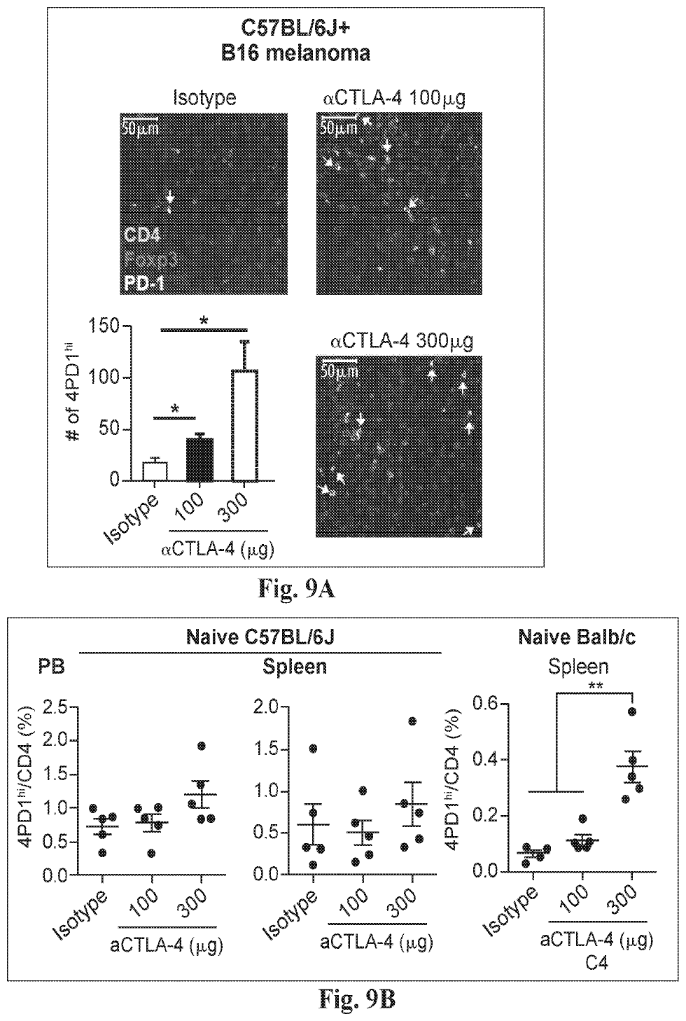

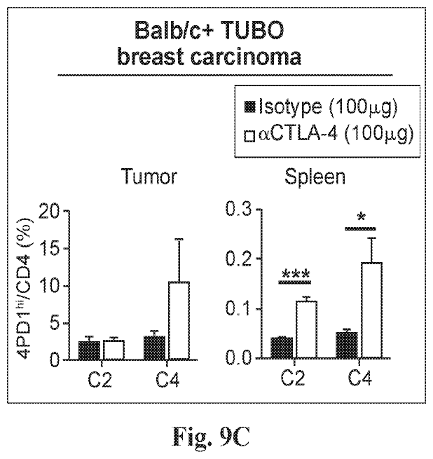

To evaluate the role of 4PD1^{hi} in the development of anti-tumor immune responses in vivo, we monitored this cell population in cancer patients treated with immune checkpoint blockade. To detect human PD-1, we employed a mAb whose binding is not cross-blocked by the therapeutic αPD-1 Abs nivolumab or pembrolizumab (FIG. 7A). In metastatic NSCLC patients, we found that nivolumab monotherapy reduced peripheral 4PD1^{hi} (FIG. 8A, nivo3, n=10). Interestingly, addition of a relatively low (FIG. 8A, nivo1+ipi1, n=11) or higher dose (FIG. 8A, nivo1+ipi3, n=8) of the αCTLA-4 ipilimumab to nivolumab produced proportional increases in circulating 4PD1^{hi} compared to the patients treated with nivolumab monotherapy (FIG. 8A). We thus explored in mice the capability of αCTLA-4 monotherapy to increase 4PD1^{hi} in a dose-dependent manner, by treating with 100 μg (standard dose in mice) or a higher amount (300 μg) of αCTLA-4 (FIG. 8B). Aligned with the observation in cancer patients (FIG. 8A), in B16-bearing mice, increases in circulating and intra-tumor 4PD1^{hi} were proportional to the dose of αCTLA-4 administered (FIG. 8B, FIG. 9A), and peaked very rapidly after treatment (FIG. 8B). Furthermore, in different mouse strains (C57BL/6J and Balb/c) we observed that the presence of tumor contributes to anti-CTLA-4-mediated induction of 4PD1^{hi}. In contrast to tumor-bearing mice, 4PD1^{hi} did not significantly increase upon treatment with the standard anti-CTLA-4 dose (100 μg) in non-tumor-bearing animals (FIG. 9B-9C).

We further confirmed the results achieved in NSCLC patients in larger cohorts of metastatic melanoma patients treated with ipilimumab (FIG. 8C, αCTLA-4, n=47) or the αPD-1 pembrolizumab (FIG. 8C, n=52, 50/52 upon relapse on ipilimumab). αCTLA-4 increased circulating 4PD1^{hi}, while administration of αPD-1 reduced their frequency (FIG. 8C). We further substantiated the capability of αPD-1 (pembrolizumab) to down-regulate 4PD1hi in an independent cohort of melanoma patients (Huang et al., 2017) (FIG. 8F). These data indicate that αCTLA-4 and αPD-1 modulate 4PD1^{hi} frequency in opposing directions in cancer patients, and suggest that combining different dosages (as in FIG. 8A) may differentially affect 4PD1^{hi}, with αPD-1 being able to antagonize the effects of αCTLA-4 as long as αCTLA-4 dose is not in relative excess.

### Example 4. 4PD1hi are a Biomarker of Activity of Immune Checkpoint Blockade

To assess whether levels of 4PD1^{hi} constituted a pharmacodynamic biomarker of αPD-1 therapeutic activity, we compared overall survival (OS) of advanced melanoma patients according to 4PD1^{hi} frequency and modulation during pembrolizumab treatment (FIG. 8C right panel). Table 1 shows the post-therapy 4PD1^{hi} levels and clinical benefit in pembrolizumab-treated advanced melanoma patients.

TABLE 1

| | | Haz | | Cox |
|---|---|---|---|---|
| Variable | n | Ratio | 95% CI | p value |
| 4PD1^{hi} % (3 wks | 52 | 1.4 | (1.16, 1.70) | .0005 *** |
| post-Tx) | (24 deaths) | | | |
| Post/Pre-Tx FoldChange | 52 | 4.4 | (1.03, | .046 * |
| in 4PD1^{hi} % | (24 deaths) | | 19.14) | |

We assessed correlation of 3 weeks post-treatment 4PD1^{hi} frequency (3 wks post-Tx, end of 1^{st} treatment cycle) and 4PD1^{hi} fold change relative to baseline (post-/pre-Tx 4PD1^{hi}) with overall survival in advanced melanoma patients treated with pembrolizumab (n=52, FIG. 8C). Hazard ratios (risk of death, Haz Ratio) for 4PD1^{hi} frequencies and 4PD1^{hi} fold reductions and associated p values calculated with the Cox regression model using continuous variables are reported. We found that elevated 4PD1^{hi} frequencies and/or lack of significant 4PD1^{hi} down-modulation after PD-1 blockade resulted in a significantly higher risk of death (Table 1, FIG. 8G-8H). These patients should receive stronger treatment with PD-1 blockade or other therapies that down-regulate 4PD1^{hi}.

As intra-tumoral 4PD1^{hi} modulation is paralleled by similar changes in PB, we could monitor these effects in cancer patients during checkpoint blockade treatment in association with the clinical outcome. In melanoma patients treated with pembrolizumab after progression on ipilimumab, who thus started with greater amounts of 4PD1^{hi}, lack of efficient reduction of 4PD1^{hi} after PD-1 blockade was associated with a significantly higher risk of death, indicating that 4PD1^{hi} levels constitute a prognostic factor in cancer patients treated with immunotherapy. Given that 4PD1^{hi} are modulated by checkpoint blockade in a dose dependent manner, such a biomarker may be valuable to guide the definition of optimal dosage/schedule of these treatments across different malignancies. This may be particularly useful as activity and tolerability of these therapies can vary depending on the tumor type, and determining the optimal regimen in each individual case is a clinical priority (Hellmann et al., 2016; Larkin et al., 2015; Postow et al., 2015; Rizvi et al., 2015; Wolchok et al., 2013).

To confirm the therapeutic impact of targeting 4PD1^{hi} in mice, we tested the effects of PD-1 blockade in B16-bearing mice treated with αCTLA-4 and the anti-melanoma vaccine VRP-TRP2, so as to recapitulate the setting with increased 4PD1^{hi} level and suboptimal therapeutic effects that we previously described (Avogadri et al., 2014). The triple combination treatment (VRP-TRP2+αCTLA-4+αPD-1) promoted tumor shrinkage and durable tumor control compared to the individual Abs plus the vaccine (FIG. 8D) and reduced intra-tumor 4PD1^{hi} (FIG. 8E), as assessed by the anti-PD-1 mAb RMP1-30 that is not cross-blocked by the therapeutic clone RMP1-14 (FIG. 7B). VRP-TRP2 plus αPD-1 alone, while preventing an increase in 4PD1^{hi}, promoted intra-tumor accumulation of T_{regs} (FIG. 8E). Concomitant CTLA-4 and PD-1 inhibition in the triple combination treatment counteracted reciprocal induction of 4PD1^{hi} and T_{regs} by each checkpoint blockade therapy (FIG. 8E), thus providing one possible explanation for its increased therapeutic effects (FIG. 8D).

In B16-bearing mice vaccinated with VRP-TRP2, therapeutic improvement with the addition of PD-1 blockade to αCTLA-4 was associated with reciprocal control of 4PD1^{hi} and T_{reg} expansion, with CTLA-4 blockade inducing 4PD1^{hi} cells but not intra-tumor T_{regs}, and PD-1 blockade enhancing intra-tumor T_{regs}, while preventing 4PD1^{hi} induction. Preclinical evidence points to the capacity of PD-1 to control T_{reg} homeostasis by restraining T_{reg} peripheral conversion (Ellestad et al., 2014) as well as TFR development (Sage et al., 2013). In tumor-bearing hosts, PD-1 blockade may thus remove this control and promote the generation of tumor-associated T_{regs}. As the PD-1 blocking Abs used in this study do not promote depletion of the targeted cells, 4PD1^{hi} loss during PD-1 blockade may instead result from enhanced cell death due to over-stimulation in the absence of PD-1 regulatory signals, especially with concurrent CTLA-4 blockade. Alternatively, αPD-1 may antagonize 4PD1^{hi} development by increasing T_{regs}, which in turn limit T-cell priming (Sage et al., 2013) and thus 4PD1^{hi} induction. In support of the negative effects of PD-1 blockade on the B-cell stimulatory 4PD1^{hi} pool, we found that anti-tumor humoral immunity is hampered in mice treated with PD-1 blockade.

### Example 5. Selective PD-1 Pathway Blockade in 4PD1hi Counteracts Their Inhibitory Function

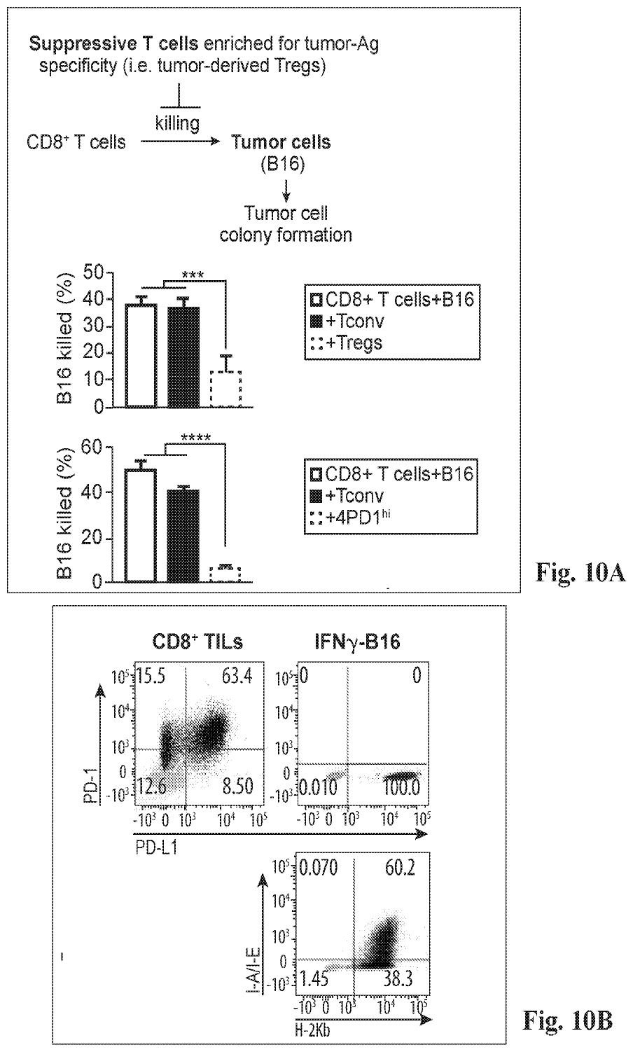

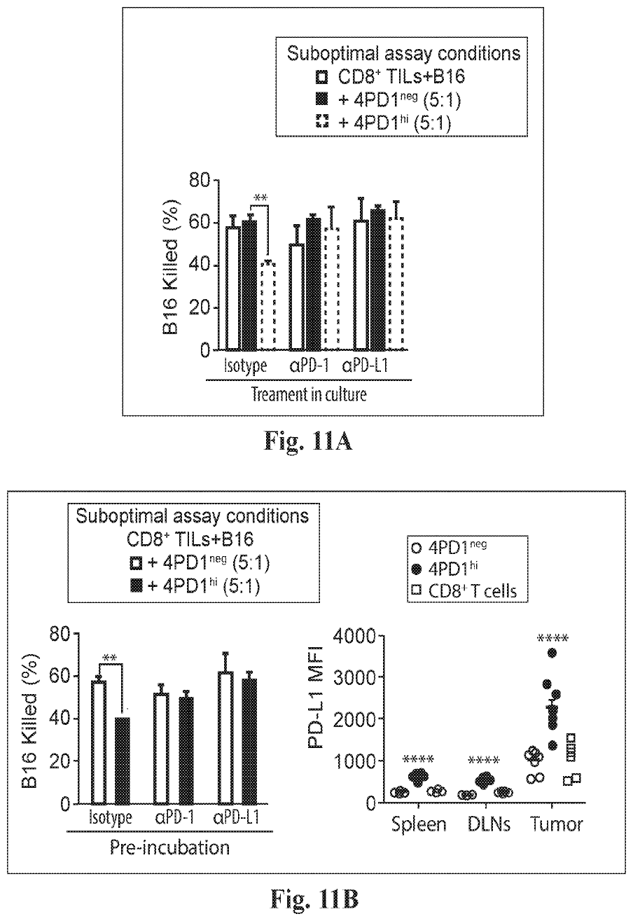

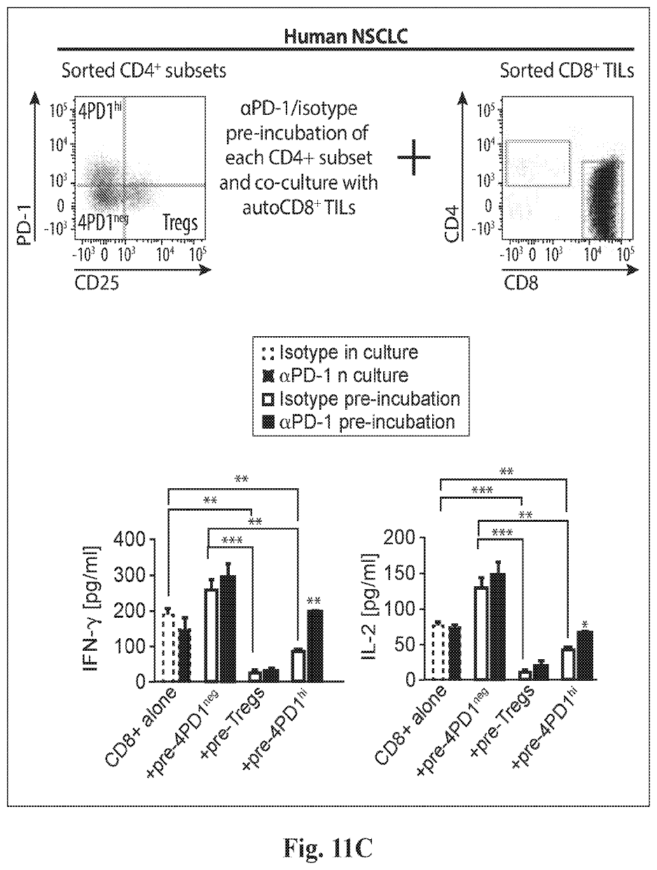

To determine whether PD-1 constituted a functional target, in addition to being a key phenotypic marker of 4PD1^{hi}, we tested the effect of PD-1 pathway blockade on 4PD1^{hi} inhibition of T-cell tumoricidal function in a 3D killing assay. In this in vitro system, tumor-antigen specific CD8⁺ T cells are co-cultured with tumor cells and suppressive T cells enriched for tumor-antigen specificity (i.e., tumor-derived T_{regs}) in order to evaluate the inhibition of CD8⁺ T-cell-mediated tumor killing (Budhu et al., 2010) (FIG. 10A). In this setting, we observed significant inhibition of CD8⁺ T cell-mediated B16 killing when tumor-derived 4PD1^{hi} where used in place of T_{regs} (FIG. 10A). We thus employed the same assay to test the effects of PD-1 or PD-L1 blockade on 4PD1^{hi} inhibitory activity. To enable a parallel analysis of 4PD1^{hi} treated in multiple conditions, we reduced the number and ratios of 4PD1^{hi} cells relative to CD8⁺ T cells to use in each culture. Even in this suboptimal setting, 4PD1^{hi} limited B16 killing (FIG. 11A). Importantly, PD-1 or PD-L1 blockade restored CD8⁺ T-cell-mediated B16 killing in the presence of 4PD1^{hi}, but did not augment baseline CD8⁺ T-cell cytotoxicity (FIG. 11A), pointing to a functional role of PD-1/PD-L1 inhibition in 4PD1^{hi} for this effect. However, given the high PD-1 and/or PD-L1 expression on CD8⁺ TILs and B16 cells (FIG. 10B), we could not exclude a contribution from blocking the PD-1 pathway on those cells. We therefore selectively blocked PD-1 or PD-L1 with specific Abs on 4PD1^{hi}, or 4PD1^{neg} as control, before adding these cells to CD8⁺TIL-B16 co-cultures. Selective blockade of either PD-1 or PD-L1 on 4PD1^{hi} was sufficient to abolish their inhibitory function (FIG. 11B top panel), and we found that 4PD1^{hi} also overexpressed PD-L1, particularly at the tumor site (FIG. 11B bottom panel). This suggests that the PD-1 pathway mediates 4PD1^{hi} inhibitory activity.

To confirm these findings in the human setting, we tested whether PD-1 blockade on 4PD1^{hi} from human tumors affects their inhibitory function. In the absence of TILs and clonogenic tumor cell lines from the same patients to perform 3D killing assays, we adapted the standard suppression assay described above to measure activation of T cells co-cultured with PD-1-blocked or control 4PD1^{hi}. Human NSCLC-derived 4PD1^{hi}, T_{regs}, and 4PD1^{neg} were pre-incubated with saturating doses of αPD-1 or the matched isotype IgG control and, after washing, co-cultured with stimulated autologous target CD8⁺ TILs (FIG. 11C top panels). We found increased IFNγ and IL-2 production in culture of CD8⁺ TILs with PD-1-blocked 4PD1^{hi} (FIG. 11C bottom panels), suggesting that blocking PD-1 on 4PD1^{hi} may favor the development of cytotoxic anti-tumor T-cell responses in vivo. To fully control for the potential spillover of αPD-1 from pre-incubated cells and direct engagement of PD-1 on target CD8⁺ TILs, we monitored the maximum effects that direct PD-1 blockade could provide on target cells by culturing them with αPD-1 (FIG. 11C bottom panels, filled gray bars) or control IgG (FIG. 11C bottom panels, open gray bars) in parallel. Even upon direct culture with αPD-1, CD8⁺ TILs did not show a major increase in cytokine release (FIG. 11C bottom panels), thus confirming that the effects observed in the presence of PD-1-blocked 4PD1^{hi} were primarily due to 4PD1^{hi}-specific functional reprogramming.

### Example 6. 4PD1^{hi} Express a T_{FH}-Like Phenotype

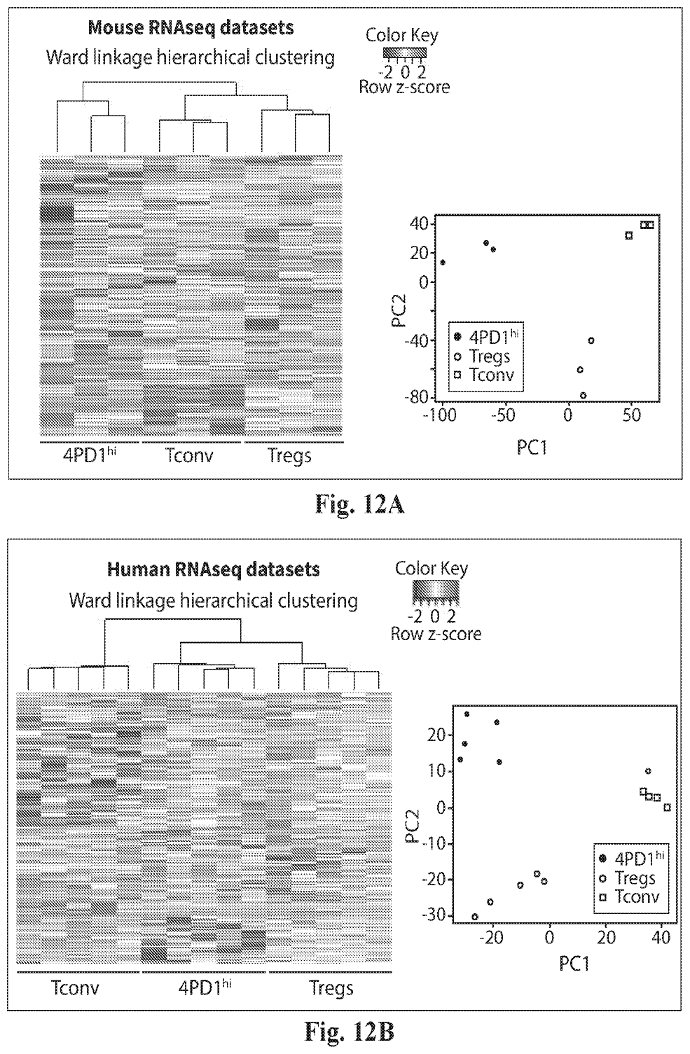

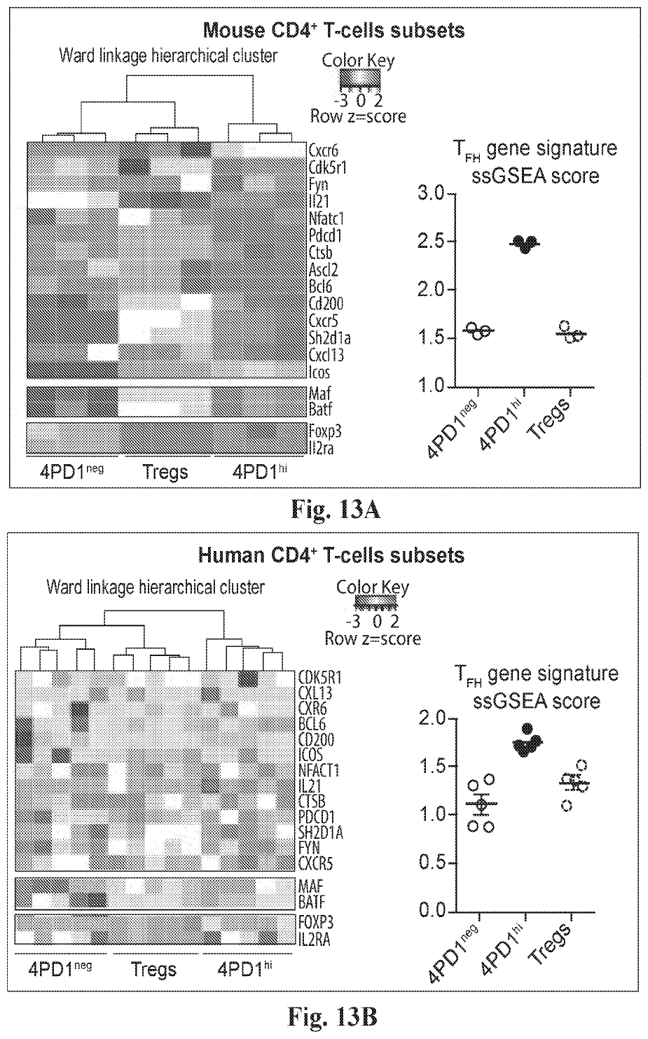

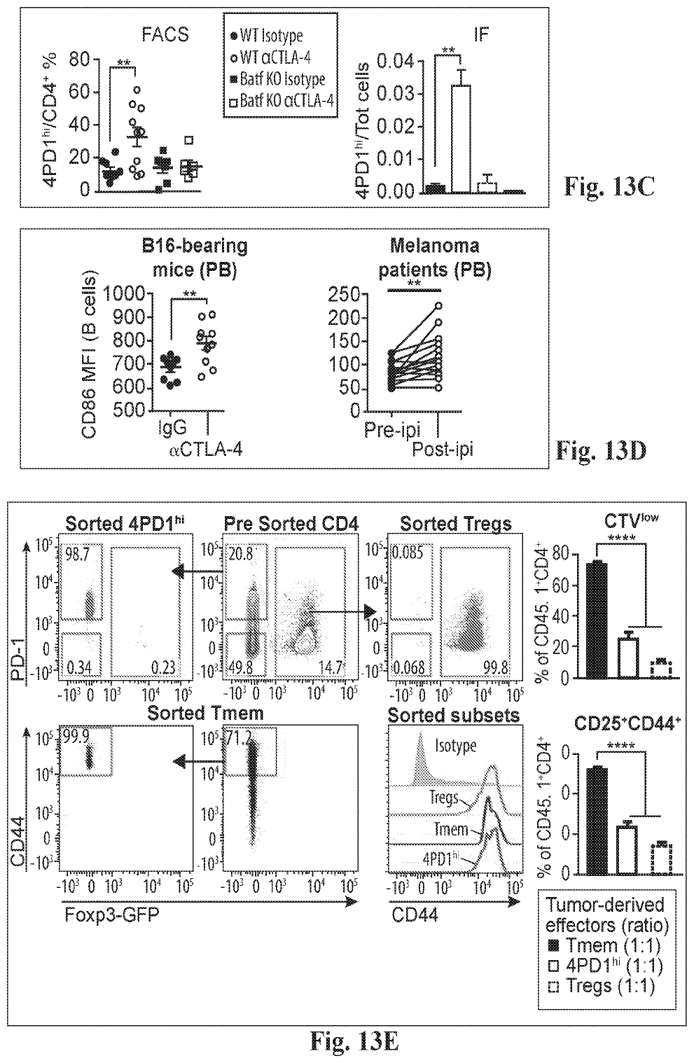

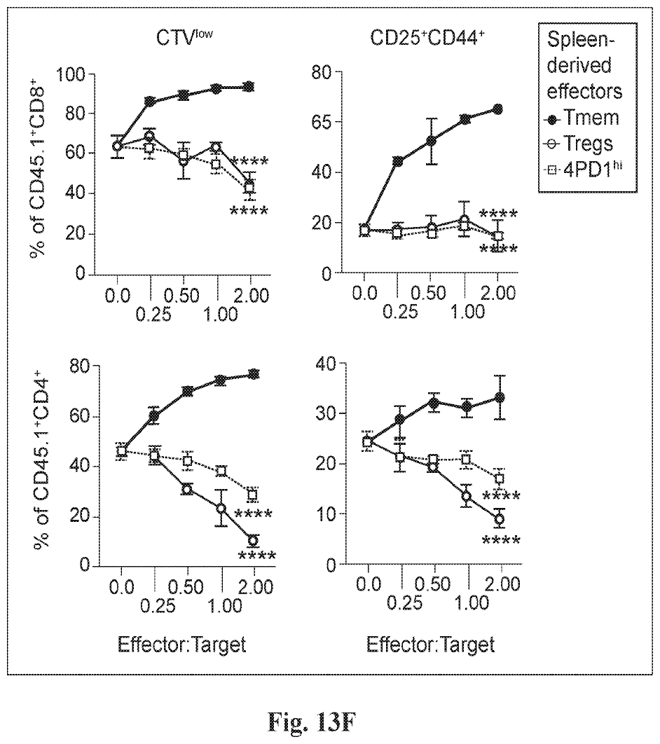

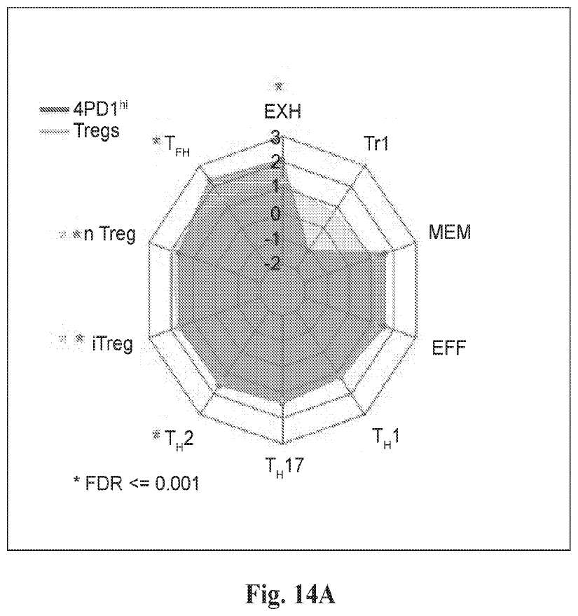

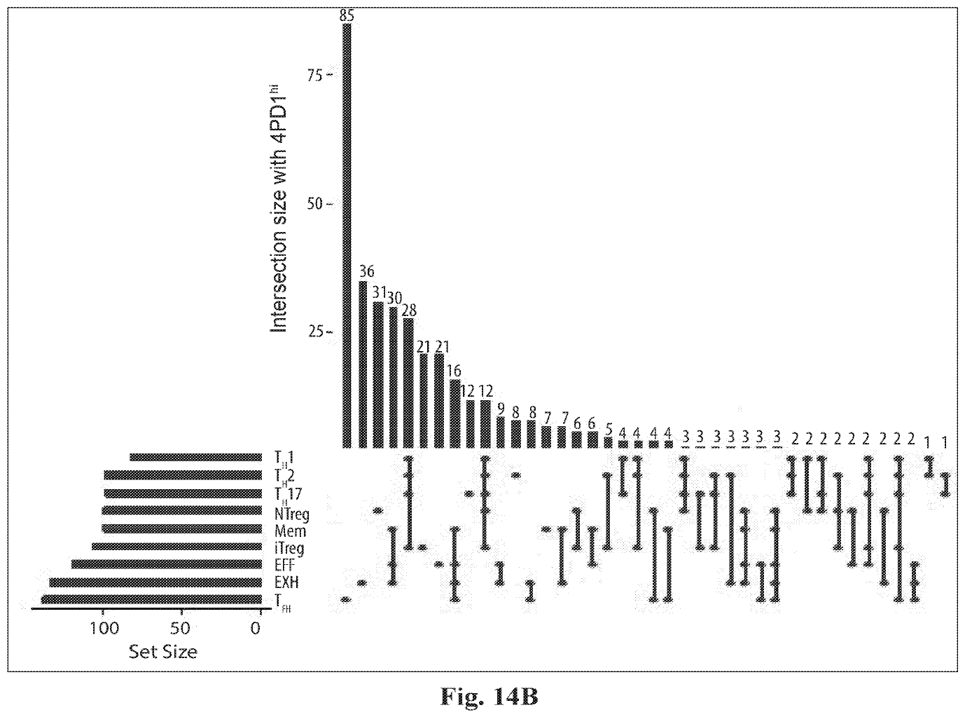

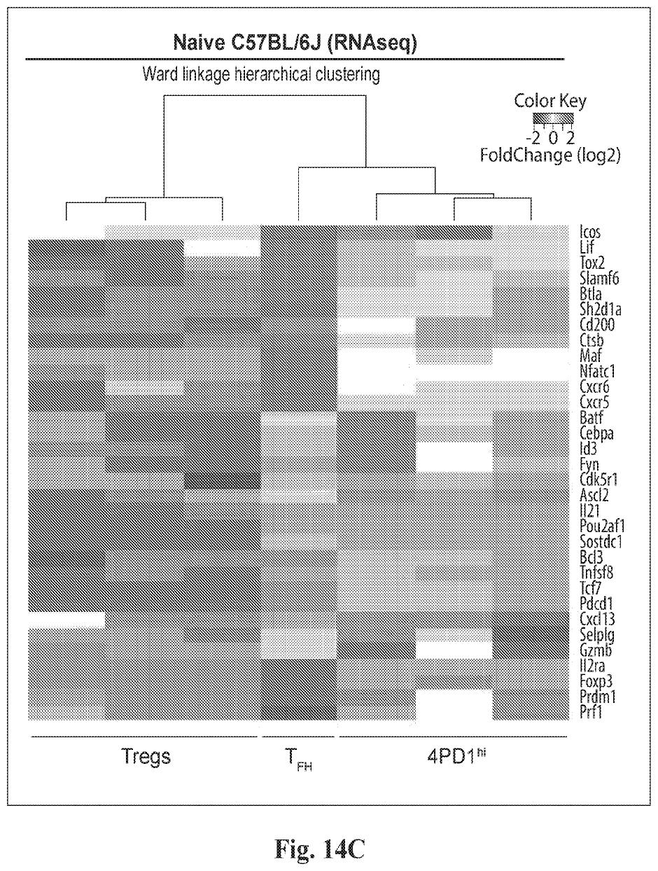

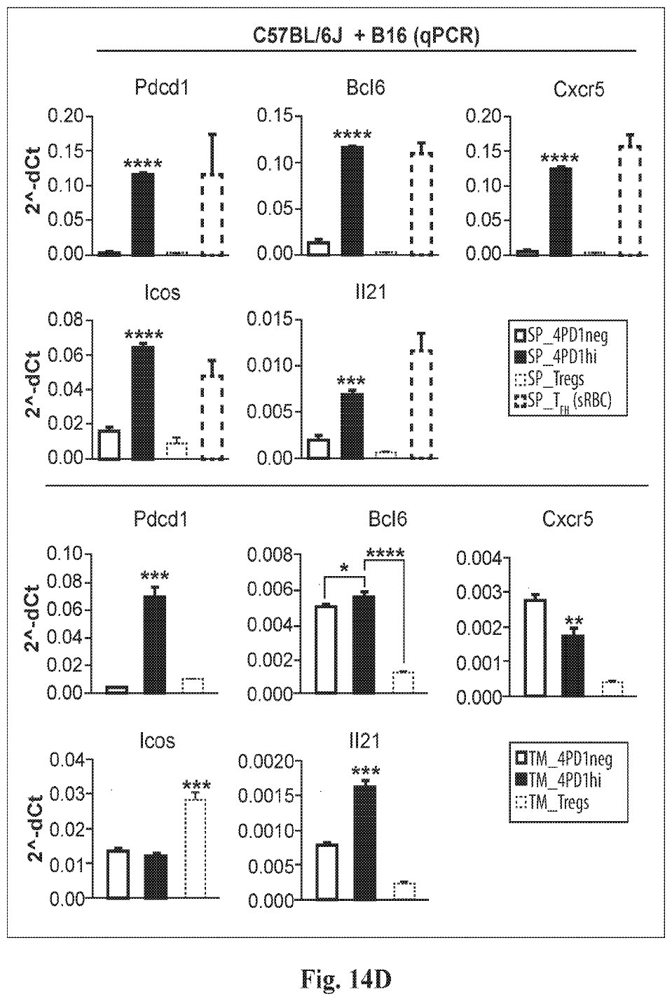

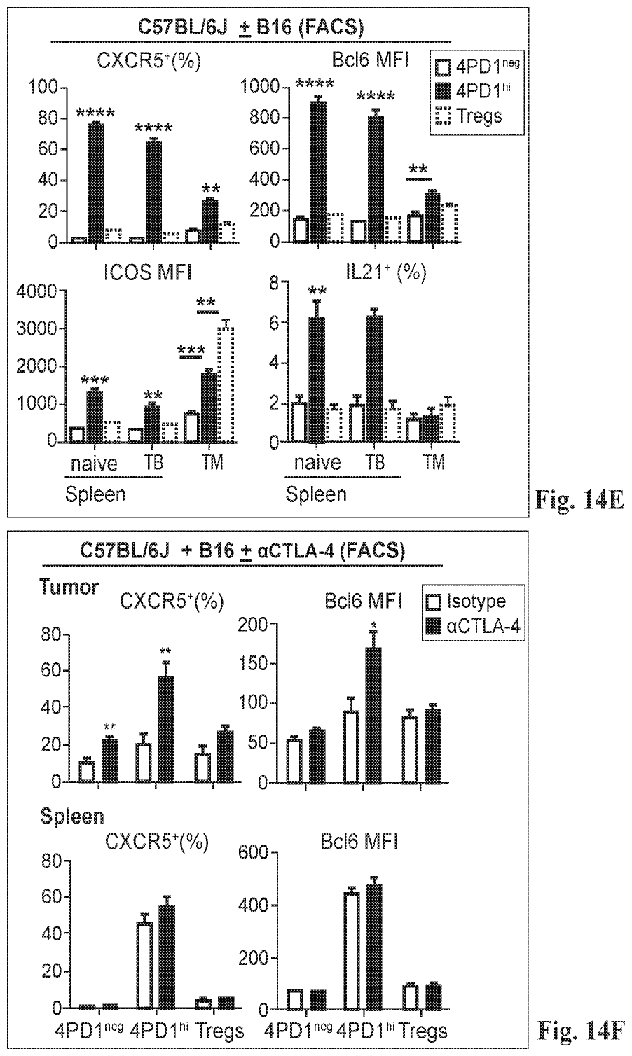

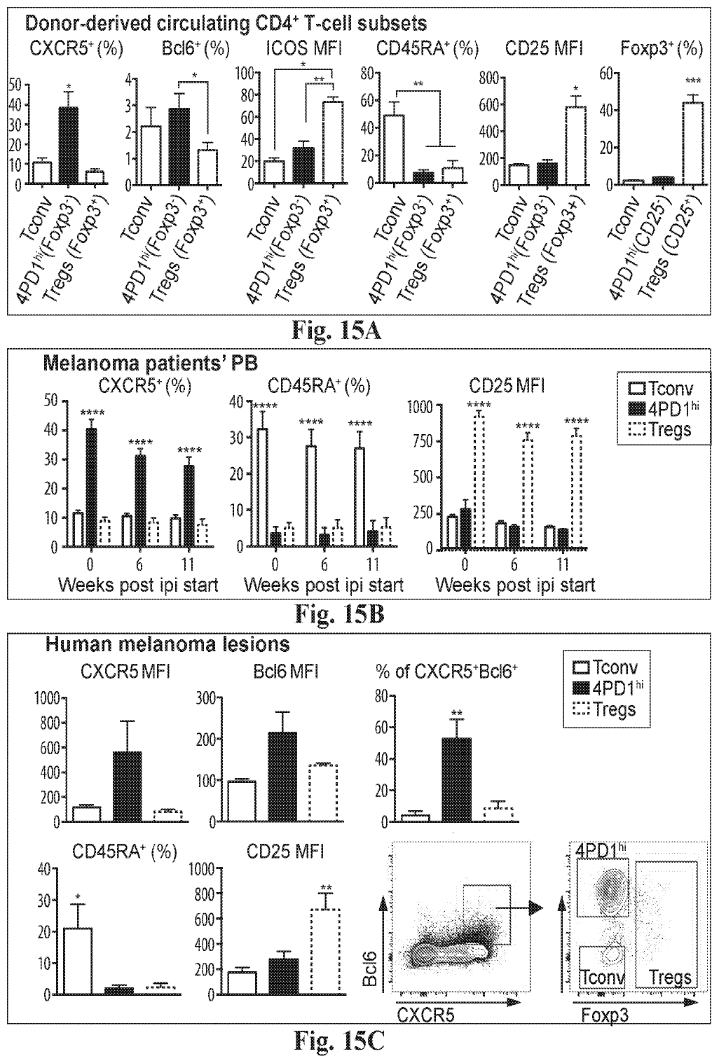

To determine whether 4PD1^{hi} constitute a distinct inhibitory T-cell entity, we compared RNAseq gene expression profiles of mouse and human 4PD1^{hi}, T_{regs}, and 4PD1^{neg} previously tested in suppression assays (FIG. 3B, FIG. 4A-4B, FIG. 5A, FIG. 6A). Unsupervised hierarchical clustering and principal component analysis (PCA) of variably expressed genes showed that these three CD4⁺ T-cell subsets are transcriptionally distinct populations both in mice and humans (FIG. 12). Gene set enrichment analysis of gene signatures from known CD4⁺ T-cell subsets in 4PD1^{hi} revealed extensive overlap with T_{FH} and exhausted T cells (FIG. 14A). However, the greatest number of genes shared with 4PD1^{hi} were unique to the T_{FH} phenotype (FIG. 14B). Accordingly, 4PD1^{hi} and conventional T_{FH} transcriptomes (Miyauchi et al., 2016) showed overlapping profiles when a comprehensive set of genes previously found differentially expressed (up-regulated and down-regulated) in bona fide T_{FH} (Choi et al., 2015; Kenefeck et al., 2015; Liu et al., 2012; Miyauchi et al., 2016) was analyzed (FIG. 14C). Importantly, both mouse and human 4PD1^{hi} were accurately distinguished from 4PD1^{neg} and T_{regs} by genes typically overexpressed in T_{FH} (Kenefeck et al., 2015; Sahoo et al., 2015) (FIG. 13A-13B). D25 and Foxp3 were selectively overexpressed in T_{regs} in this analysis (FIG. 13A-13B, FIG. 14C, FIG. 15A-C).

T_{FH} are a specialized subset of CD4⁺ T cells, generally defined by CXCR5, BCL6, ICOS, and PD-1 expression, which assist germinal center (GC) B cells to produce high-affinity Abs, in particular through the release of IL-4 and IL-21 (Crotty, 2014; Sahoo et al., 2015). The chemokine receptor CXCR5 and transcription factor BCL6 are responsible for directing and maintaining T_{FH} in the B-cell zone in secondary lymphoid organs, where they exert B-cell helper functions; whereas the co-stimulatory molecule ICOS and co-inhibitory receptor PD-1, which indicate a status of mature/antigen-experienced cells, regulate T_{FH} activation levels (Akiba et al., 2005; Cubas et al., 2013; Sage et al., 2013). In both mice and humans, T_{FH} can down-regulate BCL6 and CXCR5, exit GCs and recirculate in the periphery as memory T_{FH} (Hale and Ahmed, 2015; He et al., 2013; Sage et al., 2014a), highlighting the plasticity of T_{FH} phenotype according to anatomic location. The above data are consistent with multiple observations that circulating CD4⁺CXCR5⁺ T cells mirror active T_{FH} responses in secondary lymphoid organs (He et al., 2013; Tangye et al., 2013). T_{FH} are also characteristically defined by the lack of IL2rα (CD25) expression, as IL-2 is a potent inhibitor of their differentiation (Ballesteros-Tato et al., 2012; Johnston et al., 2012). Our findings that 4PD1^{hi} express an effector memory phenotype, lack CD25 and Foxp3 expression, and expand preferentially in secondary lymphoid organs were all in agreement with these T_{FH} features. Consistently, T_{FH} markers were generally expressed at higher levels in 4PD1^{hi} than in T_{regs} and 4PD1^{neg} from mice (FIG. 14D-14F), healthy donors and cancer patients (FIG. 15A-15C). However, outside of secondary lymphoid organs, such as in PB and tumor, 4PD1^{hi} did not always co-express all these T_{FH} markers at significantly higher levels, with ICOS as an example being preferentially expressed by T_{regs} in those anatomic locations (FIG. 14D-14E, FIG. 15A). This would point to a phenotype of GC-experienced T_{FH} in peripheral 4PDhi, which is distinguished by reduced expression of Bcl6, CXCR5 and ICOS (Hale and Ahmed, 2015; He et al., 2013; Sage et al., 2014a). Interestingly, in B16-bearing mice, CTLA-4 blockade up-regulated CXCR5 and Bcl6 in intra-tumor 4PD1^{hi} (FIG. 14F).

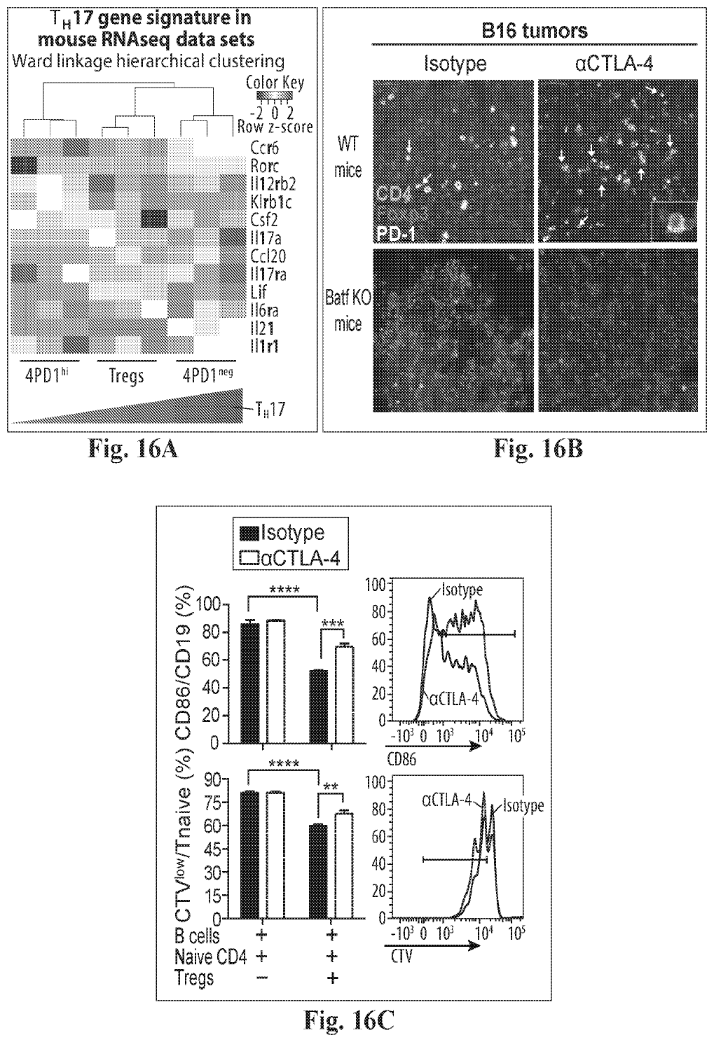

To further explore the potential link between 4PD1^{hi} and T_{FH} lineage, we tested whether anti-CTLA-4 could still increase 4PD1^{hi} in tumor-bearing mice genetically engineered to lack T_{FH} development but without any alteration in PD-1 expression. Among the few models where T_{FH} are constitutively absent, Batf KO mice were the only ones available in a C57Bl/6J-matched background with no major defects, where we could test this hypothesis (Ma et al., 2012; Sahoo et al., 2015). Expression of basic leucine zipper transcription factor ATF-like (Batf) is restricted to the hematopoietic system, where it guides B-cell class-switch recombination and T_{FH} development by directly inducing expression of AID in B cells and Bcl6 and Maf in T_{FH} (Murphy et al., 2013). Therefore, Batf KO mice have profound defects in GC reactions, but a functional T-bet-IFNγ axis and normal PD-1 expression (Murphy et al., 2013). Even though T_{H}17 differentiation is also defective in Batf KO mice (Murphy et al., 2013), the fact that 4PD1^{hi} did not preferentially express the T_{H}17-lineage-defining genes Rorc and Il 17a (FIG. 16A) quite confidently suggested that eventual differences in 4PD1^{hi} modulation in Batf KO mice could not be ascribed to hampered T_{H}17 differentiation. According to our hypothesis, CTLA-4 blockade could no longer increase intra-tumor 4PD1^{hi} in B16-bearing Batf KO mice (FIG. 13C, FIG. 16B).

We thus questioned how CTLA-4 blockade increases 4PD1^{hi} and reasoned that this effect could be mechanistically linked to inhibition of the CTLA-4-mediated control of CD86 expression on APCs (in particular B cells), which is also responsible for T_{reg} suppression of T_{FH} expansion (Hou et al., 2015; Wing et al., 2014). In line with this hypothesis, αCTLA-4-treated B16-bearing mice and melanoma patients showed CD86 up-regulation on circulating B cells together with increased 4PD1^{hi} frequencies (FIG. 13D), suggesting that these effects may be interdependent in vivo. Furthermore, we found that the αCTLA-4 Ab used in our in vivo experiments was able to counteract inhibition of CD86 expression on B cells and proliferation of naïve T cells co-cultured with T_{regs} as source of CTLA-4 (FIG. 16C). However, acquisition of suppressive function was not a general feature of all antigen-experienced CD4⁺Foxp3⁻ T cells induced upon CTLA-4 blockade. In fact, CD44⁺ antigen-experienced PD-1-negative CD4⁺Foxp3⁻ T cells (Tₘₑₘ) from the periphery or the tumor of aCTLA-4-treated mice were able to sustain T-cell proliferation and activation in contrast to 4PD1^{hi} and T_{regs} (FIG. 13E-13F).

We next asked whether blockade of T_{FH} responses could also reduce 4PD1^{hi} and in turn favor anti-tumor immunity. To test this hypothesis in a clinically relevant setting, we pharmacologically blocked Bcl6 with a selective inhibitor (Cerchietti et al., 2010). This strategy proved effective in controlling 4PD1^{hi} both in the periphery and at the tumor site, and modestly (but significantly) delayed tumor growth even in the context of CTLA-4 blockade and high baseline 4PD1^{hi} frequencies. Interestingly, Bcl6 inhibition, while reducing intra-tumor 4PD1^{hi}, favored intra-tumor T_{reg} expansion, as observed with PD-1 blockade (FIG. 8E). The anti-tumor activity of Bcl6 inhibition observed in WT mice was completely lost in RAG KO mice, which lack mature T and B cells, indicating that Bcl6 inhibition was not affecting tumor growth directly. This does not exclude that additional immune-mediated mechanisms may contribute to the therapeutic effect of Bcl6 inhibition; however, we did not find significant increases in either total CD4⁺ or CD8⁺ T-cell intra-tumor infiltration upon Bcl6 inhibition.

Our results with a Bcl6 inhibitor highlight the immune-mediated therapeutic potential of pharmacologic inhibition of 4PD1^{hi} development, even in the setting of CTLA-4 blockade. Bcl6 inhibition in combination with checkpoint blockade may thus be effective against B-cell malignancies, as well as in those cases where αPD-1+αCTLA-4 do not efficiently counteract 4PD1^{hi} expansion and/or pose serious risks of excessive autoimmune side effects. As higher-affinity second-generation Bcl6 inhibitors with improved bioavailability are becoming available (Cardenas et al., 2016), this combinatorial strategy will be facilitated further.

### Example 7. Dual Opposing Immune Activity of 4PD1hi

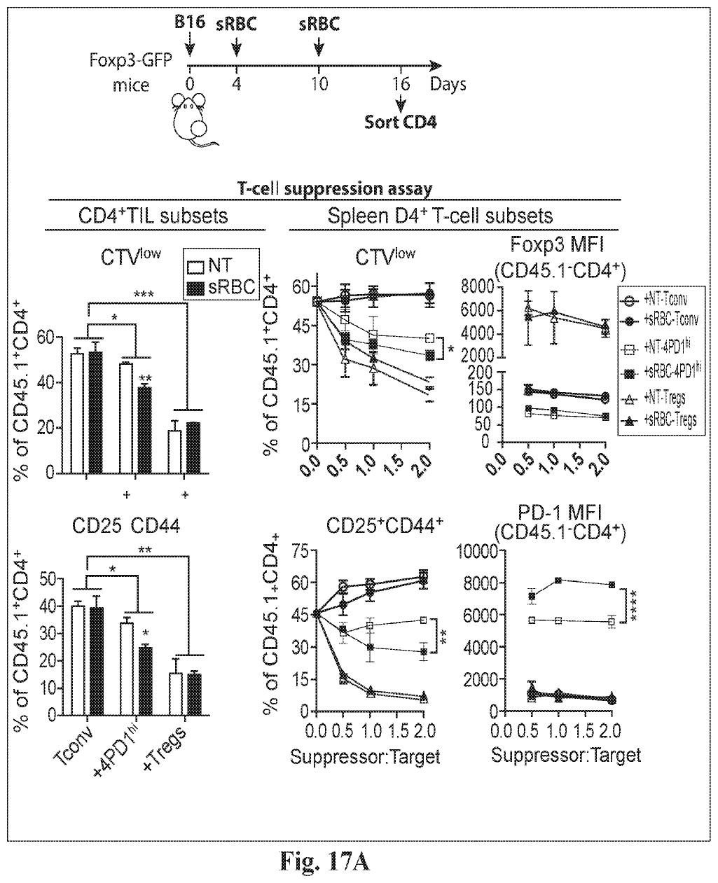

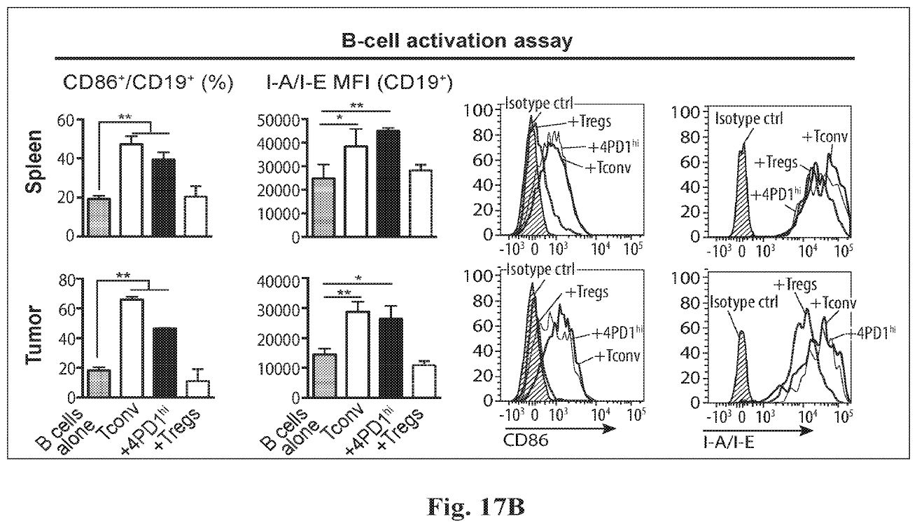

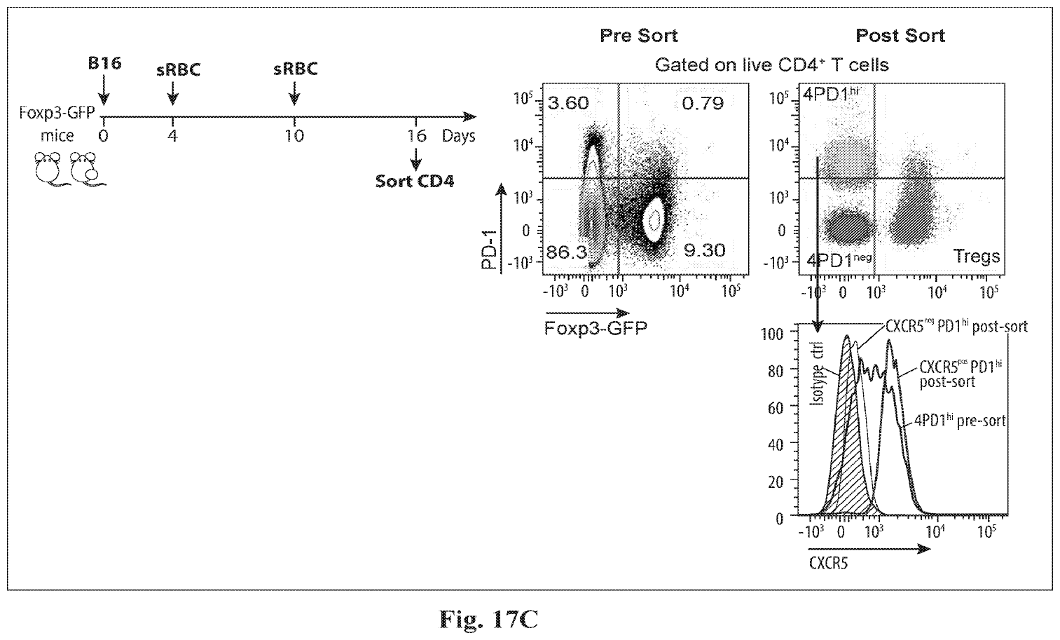

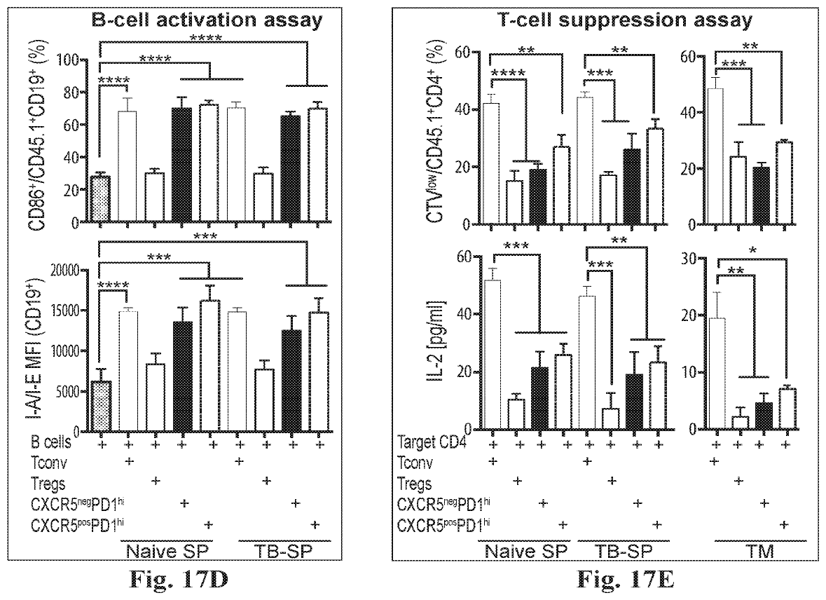

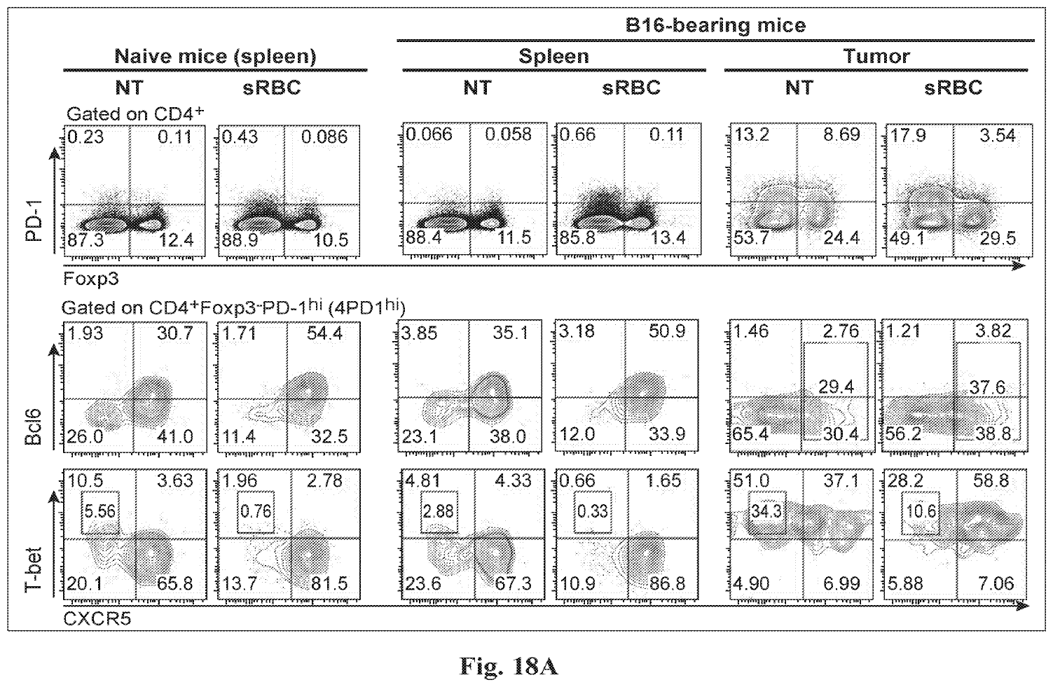

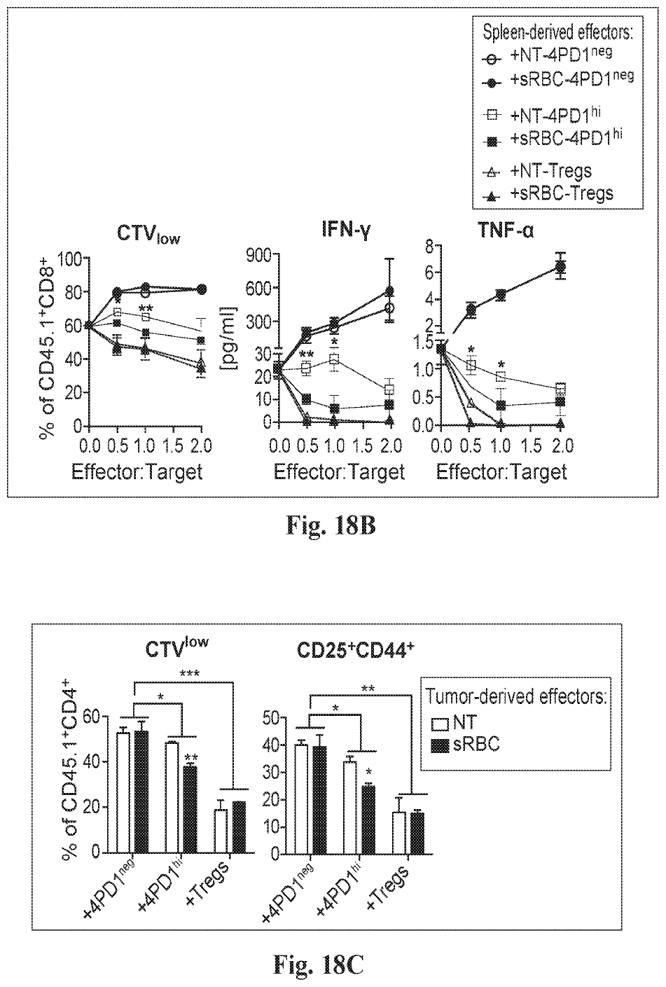

If excessive T-cell priming upon CTLA-4 blockade is at the basis of enhanced production of inhibitory 4PD1^{hi} with a T_{FH}-like phenotype, we questioned whether conventional T_{FH} responses could generate a similar T-cell population. To investigate this, we immunized mice with sheep red blood cells (sRBC) to induce GC reactions and analyzed 4PD1^{hi} modulation and function (FIG. 17A top panel). Immunization with sRBC promoted PD-1 expression in Foxp3⁻CD4⁺ T cells and T_{FH} differentiation in the 4PD1^{hi} subset, as indicated by increased Bcl6 and/or CXCR5 expression and reduced Bcl6⁻CXCR5⁻ and Tbet⁺CXCR5⁻ cell proportion within 4PD1^{hi} from both spleens and tumors in naïve and B16-bearing mice (FIG. 18A). To understand whether 4PD1^{hi} retain suppressive potential during conventional T_{FH} responses, we compared the function of 4PD1^{hi} isolated from sRBC-treated (sRBC-4PD1^{hi}) and untreated (NT-4PD1^{hi}) B16-bearing mice, and found that sRBC-4PD1^{hi} inhibited proliferation and activation of responder T cells more powerfully than NT-4PD1^{hi} (FIG. 17A, FIG. 18B-18C). Of note, stronger T-cell inhibitory activity was coupled with higher PD-1 expression levels in sRBC-4PD 1^{hi} (FIG. 17A).

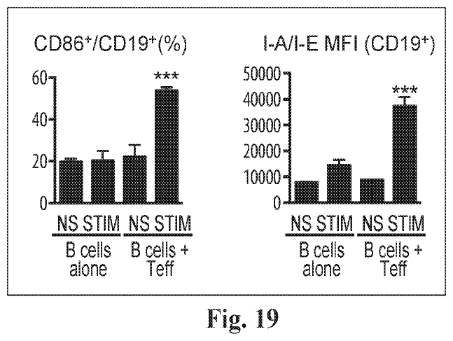

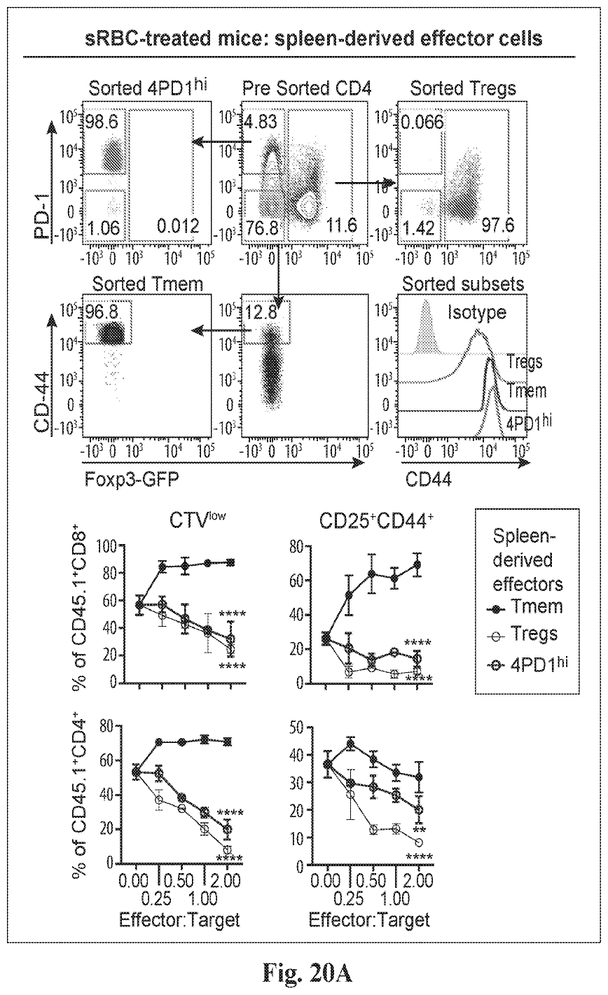

We next tested the effects of 4PD1^{hi} on B-cell activation using 4PD1^{hi} in a T-cell dependent B-cell activation assay, in which B cells mature as a function of the signals released by activated T cells over a short period of time (FIG. 19) (Wing et al., 2014). In these conditions, both spleen- and tumor-derived 4PD1^{hi} promoted B-cell activation, similar to 4PD1^{neg} and in contrast to T_{regs}, as revealed by FACS analyses of CD86 and MHC-II on B cells (FIG. 17B).

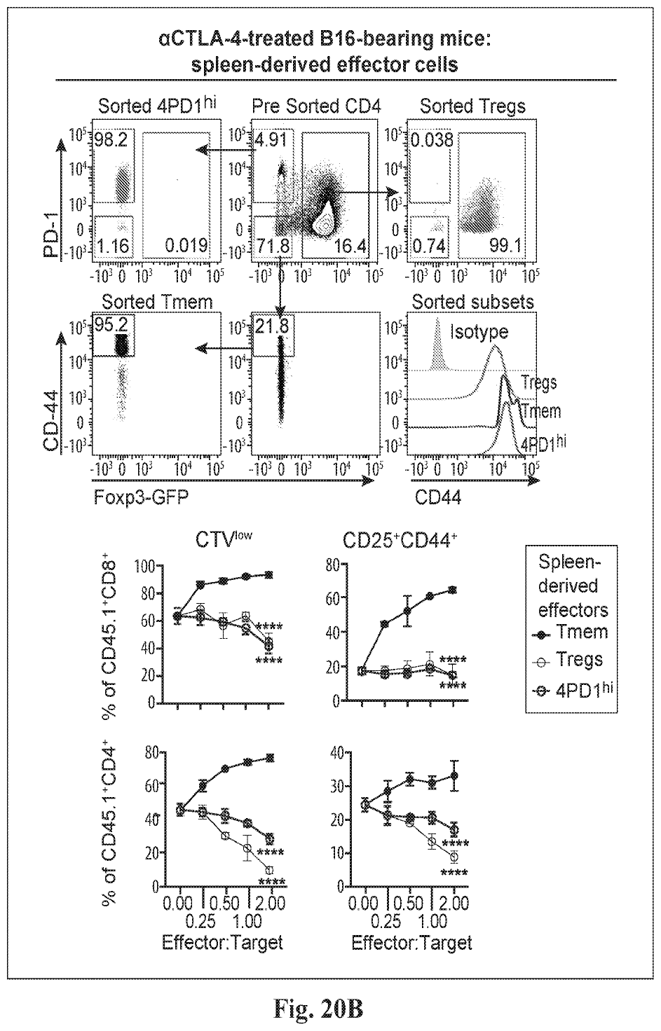

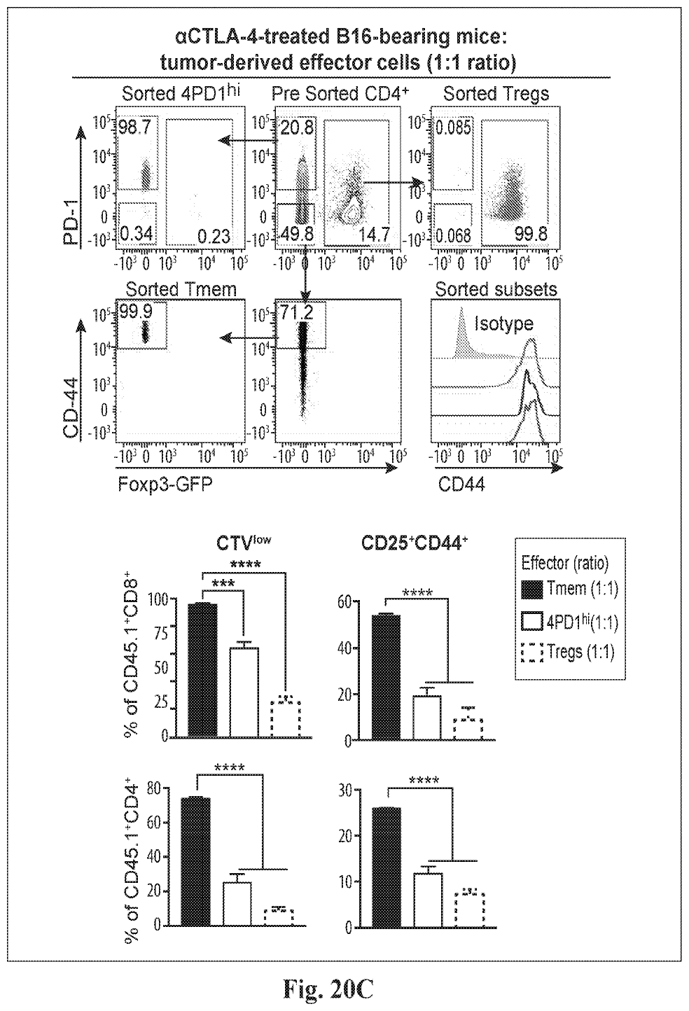

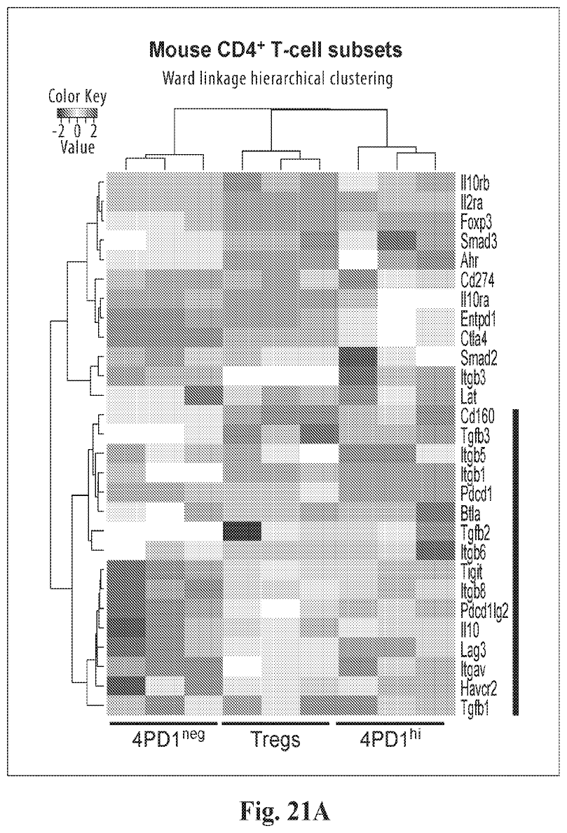

We then asked whether B-cell stimulatory and T-cell inhibitory activities were retained by the same cells within the 4PD1^{hi} pool independent of the “T_{FH} differentiation” status, and/or were modulated by the presence of tumor. To test this, we induced T_{FH} differentiation by sRBC immunization and compared functions of the CXCR5-positive (enriched in conventional T_{FH}) and CXCR5negative subsets within 4PD1^{hi} from B16-bearing and naïve mice (FIG. 17C). In either condition, CXCR5-positive and CXCR5-negative 4PD1^{hi} subsets consistently sustained B-cell activation (FIG. 17D) and limited T_{eff} functions (FIG. 17E), pointing to dual opposing immune modulating activities shared within the 4PD1^{hi} pool. Once again, the suppressive function of 4PD1^{hi} was not shared by other antigen-experienced memory T cells upon sRBC immunization, as PD-1-CD44^{hi}Foxp3⁻ Tₘₑₘ from sRBC immunized mice sustained T-cell proliferation and activation, in contrast with 4PD1^{hi} and T_{regs} from the same animals (FIG. 20A).

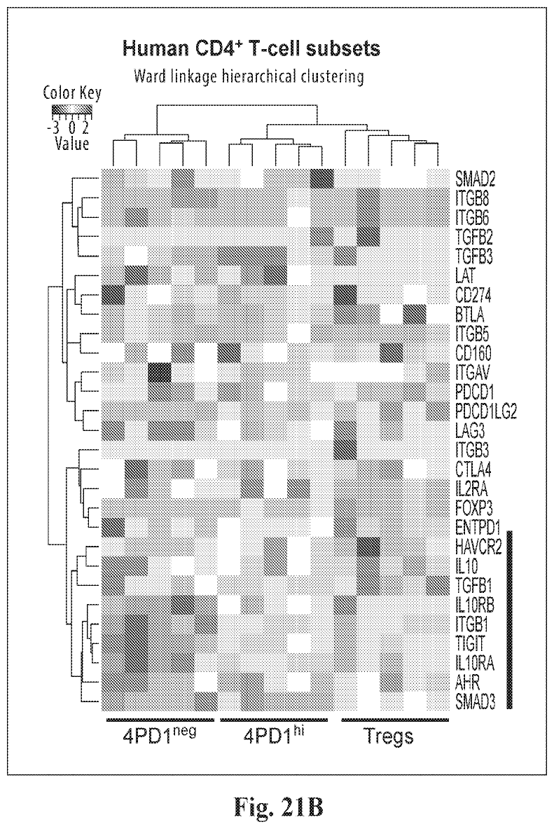

Overall, these findings suggest that exacerbated priming or T_{FH} responses (with αCTLA-4 or immunization with sRBC) can come at the expense of impaired T-cell function, which in tumor-bearing hosts may promote immune evasion. To formally prove this hypothesis, we tested CTLA-4 blockade in Sh2d1a (SAP) KO mice, which lack T_{FH} due to selective abrogation of B-T cell interactions and GC formation (Qi et al., 2008). We found that αCTLA-4 monotherapy, starting when B16 tumors are established (a regimen which is usually ineffective in wild type animals, FIG. 7D left), could still control tumor growth in Sh2d1a KO mice (FIG. 7D right), thus demonstrating that limiting T_{FH} responses can improve CTLA-4 blockade activity. The mechanism underlying this effect may be multifactorial, as indicated by the multiple immune inhibitory genes overexpressed by T_{FH}-like 4PD1hi cells, including HAVCR2, TGFB 1 and IL10, in addition to PDCD1 (FIG. 21A-21B). Dissecting the relative contribution of these immunosuppressive molecules and their interplay with the PD-1 pathway will thus be important to deepen the understanding of 4PD1^{hi} biology.

We show that anti-CTLA-4 increases CD86 expression on B cells both in vivo and in vitro and promotes CD4⁺ T-cell proliferation in vitro, thus potentially explaining how CTLA-4 blockade boosts 4PD1^{hi} generation. Previous studies reported an increase in ICOS⁺ T cells upon ipilimumab treatment (Chen et al., 2009; Ng Tang et al., 2013). As ICOS is a T_{FH} marker, these cells could include 4PD1^{hi}. However, elevation in ICOS⁺ T cells (both CD4⁺ and CD8⁺) was associated with a positive outcome of immune checkpoint blockade and was not diminished by administration of αPD-1 (Callahan et al., 2013), as opposed to what we observe for 4PD1^{hi}. This suggests that ICOS does not uniquely and specifically distinguish the inhibitory 4PD1^{hi} cells described here, and points to ICOS up-regulation as a marker of T-cell activation upon checkpoint blockade. Accordingly, in the melanoma cohort studied here, ipilimumab induced ICOS expression in all CD4⁺ T cell subsets, including 4PD1^{neg}, T_{regs}, and 4PD1^{hi}.

### Example 8. Materials and Methods

### Mice and Cell Lines

All mouse procedures were performed in accordance with institutional protocol guidelines. Wild type Balb/c and wild type, CD45.1⁺ congenic, and Batf KO C57BL/6J mice were obtained from Jackson Laboratory. Foxp3-GFP transgenic mice were generously provided by Dr. Alexander Rudensky and backcrossed to C57BL/6J at MSKCC. Pmel-1/gp100-specific CD8 TCR transgenic mice were a gift from Nicholas Restifo (NCI, Bethesda, Md.). Grm1-TG mice, where ectopic expression of the metabotropic receptor Grm1 (glutamate receptor 1) in melanocytes spontaneously drives melanomagenesis (Pollock et al., 2003), were provided by S. Chen (Rutgers, The State University of New Jersey, Piscataway, N.J.). Mice were maintained according to NIH Animal Care guidelines, under a protocol approved by the MSKCC Institutional Animal Care Committee. The B16F10 mouse melanoma cell line was originally obtained from I. Fidler (M. D. Anderson Cancer Center, Houston, Tex.) and cultured in RPMI 1640 medium supplemented with 10% inactivated FBS, 1× nonessential amino acids and 2 mM 1-glutamine. The BALB-neu derived mammary carcinoma cell line TUBO was kindly provided by Dr. G. Forni (University of Turin, Italy) and cultured in DMEM supplemented with 20% inactivated FBS, 1× nonessential amino acids and 2 mM 1-glutamine. Cell lines were maintained in a humidified chamber with 5% CO2 at 37° C. for up to 1 week after thawing before injection in mice.

### Patient Material

All patients and healthy donors signed an approved informed consent before providing tissue samples. Patient samples were collected on a tissue-collection protocol approved by the MSKCC Institutional Review Board and processed as described (Holmgaard et al., 2015).

### In Vivo Tumor Injection and Treatment

B16 melanoma cells were implanted intradermally (10⁵ cells, for tumor-growth and survival analyses) or subcutaneously in matrigel (Matrigel Matrix Growth Factor Reduced, Becton Dickinson) (2×10⁵ cells, for immune-cell infiltrate analyses) in C57BL/6J mice. Vaccination with VRP-TRP2 (AlphaVax Inc.) was performed by injection of 1×10⁶ virus-like replicon particles (VRPs) (Zappasodi and Merghoub, 2015) expressing mouse TRP2 into the plantar surface of each footpad for 3 times 1 week apart, starting 3 days after tumor implantation (Avogadri et al., 2014). Treatment with anti-CTLA-4 (clone 9D9, 100 μg or 300 μg/injection), anti-PD-1 (clone RMP1-14, 250 μg/injection), or the matched isotype IgGs (BioXcell) was started 3-4 (optimal treatment) or 6-7 days (suboptimal treatment) after tumor implantation for respectively 5 or 4 intraperitoneal (i.p.) administrations 3 days apart. Immunization with sRBC was performed i.p. with 200 μl 10% volume/volume sRBC solution (Innovative Research). The Bcl6 inhibitor 79.6 (Calbiochem) was administered daily i.p. in 10% DMSO at 50 mg/kg (Cerchietti et al., 2010). TUBO breast carcinoma cells were implanted subcutaneously in Balb/c mice (10⁶ cells/mouse) and anti-CTLA-4 treatment was started 10 days after. Animals were monitored at least twice a week and were considered tumor-free until intradermal lesions were palpable.

### FACS Analyses and Sorting

Tumors were dissociated after 30 min incubation with Liberase TL and DNAse I (Roche) to obtain single-cell suspensions. When tumor mass exceeded 0.1 gr, immune-cell infiltrates were enriched by Percoll (GE Healthcare) gradient centrifugation. Cells from tumor-draining lymph nodes and spleens were prepared by mechanical dissociation on 40 μM filters and RBC lysis (ACK buffer, Lonza). Mouse PB was collected by retro-orbital puncture and RBC were lysed with Pharm Lyse Buffer (BD Bioscences). Surface staining of mouse cells was performed after 15 min pre-incubation with anti-mouse CD16/CD32 Ab (clone 2.4G2; BD Biosciences) to block FcγR binding, with panels of appropriately diluted fluorochrome-conjugated Abs (from BD Biosciences, eBioscience or Invitrogen) against the following mouse proteins in different combinations: CD45 (clone 30-F11), CD45.1 (clone A20), CD4 (clone RM4-5), CD8a (clone 5H10), Thy1.1 (clone OX-7), B220 (RA3-6B2), CD19 (clone 1D3), PD-1 (RMP1-30), CD44 (clone IM7), CD62L (clone MEL-14), CD25 (clone PC61.5), CD86 (clone GL-1), I-A/I-E (clone M5/114.15.2), PD-L1 (clone MIH5), ICOS (clone C398.4A), CXCR5 (biotin-conjugated clone 2G8, followed by PE-/APC-labeled streptavidin staining), and a eFluor506 fixable viability dye. For intracellular staining, mouse cells were fixed and permeabilized (Foxp3 fixation/permeabilization buffer, eBioscience) and incubated with appropriately diluted PECF594-labeled anti-Bcl6 (clone K112-91, BD Biosciences), PECy7-labeled anti-Ki67 (clone B56, BD Biosciences), and FITC-labeled anti-Foxp3 (clone FJK-16s, eBioscience) Abs. Surface staining of human cells was performed in the presence of FcγR Blocking reagent (Miltenyi Biotec) with proper dilutions of fluorochrome-conjugated Abs (from BD Biosciences, eBioscience or Tonbo) against the following human proteins in different combinations: CD45 (clone HI30), CD45RA (clone HI100), CD4 (clone RPA-T4), PD-1 (clone MIH4 or J105 in anti-PD-1-treatment naïve samples), CD25 (clone MA251), ICOS (clone ISA-3), CXCR5 (clone RF8B2), CD19 (clone HIB19), and CD86 (clone FUN-1), and a eFluor506 fixable viability dye. For intracellular staining, human cells were fixed and permeabilized (Foxp3 fixation/permeabilization buffer, eBioscience) and then incubated with appropriately diluted eFuor450-labeled anti-Foxp3 (clone PCH101, eBiosciences), PECF594-labeled anti-Bcl6 (clone K112-91), and APC-labeled anti-CTLA-4 (clone BNI3, BD Biosciences) Abs.

For intracellular cytokine staining, mouse immune cells were re-stimulated with 500 ng/ml PMA and 1 μg/ml ionomycin in complete RPMI 1640 supplemented with 1 mM sodium pyruvate and 50 μM β-mercaptoethanol at 37° C. After 1 hour, 1× GolgiStop and 1× GolgiPlug (BD Biosciences) were added to the cultures and incubated for additional 4-5 hours at 37° C. Surface staining was performed after FcγR blockade by incubation with eFluor450-labeled anti-PD-1 (RMP1-30), AlexaFluor (AF)700-labeled anti-CD4, and APCCy7-labeled anti-CD45 (BD Biosciences) Abs, and eFluor506-labeled fixable viability dye (eBioscience). After 30 min incubation, cells were washed, fixed and permeabilized with the Foxp3 fixation/permeabilization buffer (eBioscience) according to the manufacturer's instructions and stained for 45 min with FITC-labeled anti-Foxp3 (clone FJK-16s) and APC-labeled anti-IL-21 (clone FFA21) Abs (eBioscience). Samples were acquired on an LSRII flow cytometer (BD Biosciences) using BD FACSDiva software (BD Biosciences) and data analyzed with FlowJo software (Tree Star).

Mouse 4PD1^{hi}, T_{regs}, and 4PD1^{neg} were sorted from Foxp3-GFP mice by using CD4-pre-enriched splenocytes (CD4 Microbeads, Miltenyi Biotec) or tumor immune infiltrate enriched by Percoll gradient centrifugation. Briefly, following incubation with anti-mouse CD16/CD32 Ab, samples were stained with PECy7-labeled anti-CD4, PETexasRed-labeled CD8, and APC-labeled anti-PD-1 Abs. DAPI was added to stained samples immediately before acquisition. To isolate CXCR5-positive and CXCR5-negative 4PD1^{hi} and conventional Tr⁻H, cell suspensions were first incubated with a biotin-conjugated anti-CXCR5 Ab, washed, and then stained with fluorochrome-conjugated surface Ab cocktail including PE-labeled streptavidin. Human 4PD1^{neg}, T_{regs}, 4PD1^{hi}, and CD8⁺ T cells were sorted upon incubation with Fc Blocking Reagent and staining with FITC-labeled anti-CD4, PE-Texas Red CD8 (clone 3B5, Invitrogen), PerCPC-eF710-labeled anti-PD-1, APC-labeled anti-CD45, and APCCy-labeled anti-CD25 Abs, and DAPI immediately before acquisition. FACS sorting was conducted on a FACSAria II cell sorter (BD Biosciences). After gating according to lymphocyte morphology, excluding doublets and dead cells, CD4⁺ T cells were sub-gated into Foxp3-GFP⁻PD-1⁻ (mouse 4PD1^{neg}), Foxp3-GFP⁺ (total mouse T_{regs}) or PD-1⁻Foxp3-GFP⁺ (conventional mouse T_{regs}), and PD1^{hi} Foxp3-GFP⁻ (mouse 4PD1^{hi}), or CD25⁻PD-1⁻ (human 4PD1^{neg}), CD25⁺ (human T_{regs}) and PD1^{hi} CD25⁻ (human 4PD1^{hi}) to sort respectively 4PD1^{neg}, T_{regs}, and 4PD1^{hi} from mouse and human tissues. Conventional T_{FH} were sorted as CD4⁺Foxp3-GFP⁻CXCR5⁺PD-1^{hi} T cells from spleens of sRBC-treated Foxp3-GFP mice.

### In Vitro Assays

A 3D collagen-fibrin gel culture system previously described (Budhu et al., 2010) was adapted to study the function of suppressive T cells. Briefly, 0.1×10⁵ viable B16F10 target cells were co-embedded into collagen-fibrin gels with 1×10⁵ or 0.5×10⁵ effector CD8⁺ T cells, alone or together with 0.25×10⁵ or 0.1×10⁵ (4:1 or 5:1 ratio) 4PD1^{neg}, T_{regs}, or 4PD1^{hi} FACS-sorted from B16F10 nodules. CD8⁺ T cells were from the tumor or in vitro cultures of gp100-primed splenocytes (5-day stimulation with gp100 peptide (AnaSpec)) from Pmel-1/gp100-specific TCR transgenic mice. B16F10 target cells were pre-incubated with 100 ng/ml IFN-γ to allow MHC-II up-regulation. Gels were lysed after 48 hours, and tumor cells were diluted and plated in 6-well plates for colony formation. After 7 days, plates were fixed with 3.7% formaldehyde and stained with 2% methylene blue before counting colonies as described (Budhu et al., 2010). Where indicated, 4PD1^{hi}, and 4PD1^{neg} as control, were pre-incubated with 10 μg/ml anti-PD-1 (clone RMP1-14) or anti-PD-L1 (clone 10F.9G2) or matched isotype IgGs (BioXcell) for 30 min on ice and after extensive washes embedded into the gels. Alternatively, PD-1/PD-L1 blocking Abs (10 μg/ml) were directly added to the gels.

Suppression assays with mouse cells were performed by incubating at the indicated ratios 4PD1^{neg}, T_{regs}, or 4PD1^{hi} from Foxp3-GFP mice with CellTrace Violet (CTV, Invitrogen)-labeled target T cells immunomagnetically purified (CD4 and CD8 Microbeads, Miltenyi Biotec) from spleens of CD45.1⁺ C57BL/6J congenic mice. Cultures were stimulated for 2-3 days with 0.5 μg/ml soluble anti-CD3 Ab and irradiated splenocytes before analyses of CTV dilution and target T-cell activation.

B-cell activation/T-cell proliferation assays (Wing et al., 2014) with CTLA-4 blockade were performed in a similar way by using, in place of irradiated splenocytes, live CD19⁺ B cells immunomagnetically purified from spleens (CD19 Microbeads, Miltenyi Biotec) of CD45.1⁺ C57BL/6J congenic mice, and treating cultures with 50 μg/ml anti-CTLA-4 (clone 9D9, BioXcell) or the matched isotype IgG.

T-cell dependent B-cell activation assays were adapted from Wing et al. (Wing et al., 2014) and performed by stimulating CD45.1⁺CD19⁺ B cells with 5 μg/ml PHA (Sigma) and 20 U/ml recombinant mouse IL-2, alone or in the presence of CD45.1⁻CD4⁺ T-cell subsets at 2:1 ratio for 2 days. B-cell activation was measured by FACS analysis of CD86 and MHC-II expression.

Suppression assays with human cells were performed by incubating 4PD1^{neg}, T_{regs}, or 4PD1^{hi} FACS-sorted from PB or tumor cell suspensions with an equal amount of CTV-labeled autologous or allogeneic donor-derived T cells. Cultures were suboptimally stimulated with anti-CD3/anti-CD28 microbeads (Dynabeads Human T-Expander CD3/CD28, ThermoFisher) for 3 days before analyses of CTV dilution and target T-cell activation. Where indicated, anti-PD-1 (generously provided by Bristol-Myers Squibb), or matched isotype IgGs (10 μg/ml) as control, was added in culture or used to pre-block PD-1 on human CD4⁺ T-cell subsets by 30 min incubation on ice before co-culturing them with target T cells.

Cytokine concentrations in culture supernatants were quantified by using either BD CBA Cytokine Kits (BD Biosciences) or Luminex-based multiplex assays according to the manufacturers' instructions (eBioscience and Millipore). Heatmaps showing cytokine production were generated in the R statistical environment using log 2-transformed cytokine concentrations.

### In Vivo Suppression Assay

4PD1^{hi} and T_{regs} were FACS-sorted from B16-bearing Foxp3-GFP transgenic mice and co-transferred with CFSE-labeled Pmel-1/gp100-specific CD8⁺ T cells, purified from the spleen of Pmel-1/gp100 TCR transgenic Thy1.1⁺ mice, at 1:1 ratio via tail vein injection into irradiated (600 cGy total body irradiation) CD45.1⁺ recipients. The day after transfer, recipient mice were immunized with intradermal administration of 2×10⁵ irradiated B16 cells to stimulate transferred T cells in vivo. Seven days later, recipient mice were sacrificed and spleens processed for FACS analysis of CFSE dilution and activation markers in Pmel-1/gp100-specific Thy1.1⁺CD8⁺ T cells.

### Immunofluorescence Staining and Image Processing

Multiplex immunofluorescence staining was performed at the Molecular Cytology Core Facility of MSKCC using the Discovery XT processor (Ventana Medical Systems), as previously reported (Yarilin et al., 2015). Briefly, tissue sections were deparaffinized with EZPrep buffer (Ventana Medical Systems) and antigen retrieval was performed with CC1 buffer (Ventana Medical Systems). Sections were blocked for 30 min with Background Buster solution (Innovex) followed by avidin/biotin blocking for 8 min. Staining was performed sequentially, starting with an anti-CD4 Ab (R&D Systems, 2 μg/ml) followed by an anti-Foxp3 Ab (eBioscience, 0.5 μg/ml), and finally an anti-PD-1 Ab (Sino Biological, 1 μg/ml). Sections were incubated with primary Abs for 5-6 hours followed by incubation with appropriate biotin-conjugated secondary Abs (Vector labs, 1:200) for 60 min. Detection was performed with Streptavidin-HRP D (part of DABMap kit, Ventana Medical Systems), followed by incubation with AF488-, or AF568-, or AF647-labeled Tyramide (Invitrogen), prepared according to manufacturer instructions with predetermined dilutions. Slides were counterstained with DAPI (Sigma Aldrich, 5 μg/ml) for 10 min. Stained slides were scanned using Pannoramic Flash (Perkin Elmer) using customized AF488, AF568, AF647, and DAPI filters to separate the channels. Relevant tissue regions were drawn using Pannoramic Viewer (3DHistech) and exported as TIFF images at full resolution (0.325 μm/pixel). Image analysis was performed using the FIJI/ImageJ software (NIH). DAPI channel was used to segment and count the number of cells in each region. Each nuclear signal was dilated appropriately to cover the entire cell. Regions of interest were drawn around each cell and matched to signals detected in other channels in order to count the number of positive cells for each individual staining as well as for double or triple staining.

### Real-Time Quantitative PCR

Total RNA was extracted from FACS-purified 4PD1^{neg}, T_{regs}, and 4PD1^{hi} by using TRIZOL reagent (Invitrogen) and reverse-transcribed into cDNA using the High Capacity cDNA Transcription kit (Applied Biosystems). Expression of the indicated transcripts was quantified with the Fluidigm Biomark™ system by using the appropriate FAM-MGB-conjugated TaqMan primer probes (Applied Biosystem) upon target gene pre-amplification according to the manufacturer's protocol. Gene expression was normalized relative to glyceraldehyde-3-phosphate dehydrogenase (GAPDH). Data were analyzed by applying the 2^{(-dCt)} calculation method.

### Spectratyping

RNA from FACS-purified 4PD1^{neg}, T_{regs}, and 4PD1^{hi} was prepared and used for cDNA synthesis. The cDNA was used as a template to amplify the TCR BV repertoire with 24 BV-specific primers and a common BC-specific primer pairs (Table 2).

TABLE 2

| Family | Sequence 5'-3' | ID | Estimated Product Size |
|---|---|---|---|
| MuBV1 | CTGAATGCCCAGACAGCTCCAAGC | 1 | 170 |
| MuBV2 | TCACTGATACGGAGCTGAGGC | 2 | 161 |
| MuBV3.1 | CCTTGCAGCCTAGAAATTCAGT | 3 | 150 |
| MuBV4 | GCCTCAAGTCGCTTCCAACCTC | 4 | 189 |
| MuBV5.1 | CATTATGATAAAATGGAGAGAGAT | 5 | 222 |
| MuBV5.2 | AAGGTGGAGAGAGACAAAGGATTC | 6 | 213 |
| MuBV5.3 | AGAAAGGAAACCTGCCTGGTT | 7 | 200 |
| MuBV6 | CTCTCACTGTGACATCTGCCC | 8 | 143 |
| MuBV7 | TACAGGGTCTCACGGAAGAAGC | 9 | 177 |
| MuBV8.1 | CATTACTCATATGTCGCTGAC | 10 | 228 |
| MuBV8.2 | CATTATTCATATGGTGCTGGC | 11 | 228 |
| MuBV8.3 | TGCTGGCAACCTTCGAATAGGA | 12 | 214 |
| MuBV9 | TCTCTCTACATTGGCTCTGCAGGC | 13 | 144 |
| MuBV10 | ATCAAGTCTGTAGAGCCGGAGGA | 14 | 135 |
| MuBV11 | GCACTCAACTCTGAAGATCCAGAGC | 15 | 151 |
| MuBV12 | GATGGTGGGGCTTTCAAGGATC | 16 | 204 |
| MuBV13 | AGGCCTAAAGGAACTAACTCCCAC | 17 | 165 |
| MuBV14 | ACGACCAATTCATCCTAAGCAC | 18 | 155 |
| MuBV15 | CCCATCAGTCATCCCAACTTATCC | 19 | 174 |
| MuBV16 | CACTCTGAAAATCCAACCCAC | 20 | 145 |
| MuBV17 | AGTGTTCCTCGAACTCACAG | 21 | 167 |
| MuBV18 | CAGCCGGCCAAACCTAACATTCTC | 22 | 169 |
| MuBV19 | CTGCTAAGAAACCATGTACCA | 23 | 161 |
| MuBV20 | TCTGCAGCCTGGGAATCAGAA | 24 | 149 |

Constant Primers

| | | |
|---|---|---|
| MuTCB3C | GCCAGAAGGTAGCAGAGACCC | 25 |
| MuTCB1up | GAGAAATGTGACTCCACCCAA | 26 |
| MuTCB1-FAM | FAM-(C)TTGGGTGGAGTCACATTTCTC | 27 |
| MuTCB1-HEX | HEX-(C)TTGGGTGGAGTCACATTTCTC | 28 |

BV-BC PCR products were subjected to a cycle of elongation (run-off) with an internal FAM- or HEX-labeled BC-primer. Each PCR product, representing a different TCR BV family, was size separated by electrophoresis using a 48-capillary 3730 DNA Analyzer (Life Technologies), and the product lengths were identified using the Peak Scanner software 2 (Applied Biosciences).

### RNA-Seq and Transcriptome Analysis

Whole transcriptome libraries were generated from RNA extracted from FACS-sorted CD4⁺ T cell subsets, amplified using the SMARTer Universal Low Input RNA Kit (Clontech), and sequenced on a Proton sequencing system using 200 bp version 2 chemistry at the Integrated Genomics Operation Core Facility at MSKCC. Briefly, after ribogreen quantification and quality control by the Agilent BioAnalyzer (RIN>7), cDNA was synthetized using the SMARTer Universal Low Input RNA Kit, according to the manufacturer guidelines, and then fragmentated with covaris E220. The fragmented sample quality and yield were evaluated with the Agilent BioAnalyzer. Subsequently, the fragmented material underwent whole transcriptome library preparation according to the Ion Total RNA-Seq Kit v2 protocol (Life Technologies), with 12-16 cycles of PCR. Samples were barcoded, template-positive Ion PITM and Ion Sphere™ Particles (ISPs) were prepared using the ion one touch system II and Ion PITM Template OT2 200kit v2 Kit (Life Technologies). Enriched particles were sequenced on a Proton sequencing system using 200 bp version-2 chemistry. An average of 70×10⁶ to 80×10⁶ reads were generated per sample.

The raw output BAM files were converted to FASTQ using PICARD (version 1.119) Sam2Fastq. Reads were then trimmed using fastq_quality_trimmer (version 0.0.13) with default settings. For analyses conducted in mouse cells, the trimmed reads were first mapped to the mouse genome using rnaStar (version 2.3.0e). The genome used was MM9 with junctions from ENSEMBL (Mus_musculus.NCBIM37.67) and a read overhang of 49. Any unmapped reads were mapped to MM9 using BWA MEM (version 0.7.5a). For analyses conducted in human cells, the genome used was HG19 with junctions from ENSEMBL (GRCh37.69_ENSEMBL) and a read overhang of 49. Any unmapped reads were mapped to HG19 using BWA MEM (version 0.7.5a). The two mapped BAM files were then merged and sorted and gene level counts were computed using htseq-count (options—s y-m intersection-strict) and the same gene models (Mus_musculus.NCBIM37.67 or GRCh37.69_ENSEMBL). Heatmaps of expressed genes were generated using log 2-transformed counts. Unsupervised hierarchical clustering was performed using hclust with Euclidean distance and Ward linkage. PCA was performed on log 2-transformed gene counts using the prcomp package (with parameters center=TRUE, scale=TRUE). ssGSEA was implemented using the GSVA (Hanzelmann et al., 2013) package in R to measure the level of enrichment of a T_{FH} gene signature (Kenefeck et al., 2015) in the different CD4⁺ T-cell subsets. ssGSEA takes as input the genome-wide transcriptional profile of a sample, and computes an overexpression measure for a gene list of interest relative to all other genes in the genome (Barbie et al., 2009). Heatmap and unsupervised hierarchical clustering of 4PD1^{hi}, T_{reg}, and previously reported conventional T_{FH} (Miyauchi et al., 2016) transcriptomes with respect to a broad list of T_{FH} differentially expressed genes (Choi et al., 2015; Kenefeck et al., 2015; Liu et al., 2012; Miyauchi et al., 2016) (Table 3) were generated with log 2-transformed counts normalized relative to the naïve T-cell dataset in each study.

TABLE 3
Gene Name

Ascl2
Batf
Bcl6
Btla
Cd200
Cdk5r1
Cebpa
Ctsb
Cxcl13
Cxcr5
Cxcr6
Foxp3
Fyn
Gzmb
Icos
Id3
Il21
Il2ra
Lif
Maf
Nfatc1
Pdcd1
Pou2af1
Prdm1
Prf1
Selplg
Sh2d1a
Slamf6
Sostdc1
Tcf7
Tnfsf
Tox2

All analyses after gene count generation were conducted in the R statistical environment (R development Core Team, 2008; ISBN 3-900051-07-0) (version 3.1.3).

Immunosuppressive genes analyzed in RNAseq data sets form mouse and human CD4+ T-cell subsets are shown in Table 4.

TABLE 4
Gene Name

BTLA
CD160
CTLA4
FOXP3
HAVCR2
AHR
IL10
IL10RA
IL10RB
LAG3
PDCD1
PDCD1LG2
CD274
TGFB1
TGFB2
TGFB3
SMAD2
SMAD3
LAT
ITGAV
ITGB1
ITGB3
ITGB5
ITGB6
ITGB8
ENTPD1
TIGIT

### Statistical Analyses

Two-sided Student's t test and 2-way ANOVA (with Bonferroni's multiple comparisons test) were used to detect statistically significant differences between groups. P values for tumor-free survival analyses were calculated with log-rank (Mantel-Cox) test. Pearson correlation test was used to analyze dependency between variables. The Cox regression model was used to calculate significant hazard ratios of continuous variables. Statistical analyses were performed on the Prism 7.0a software (GraphPad Software) version for Macintosh Pro personal computer. *p<0.05, **p<0.01, ***p<0.001, ****p<0.0001.

### Data Availability

The datasets generated in this study have been submitted to the GEO (Gene Expression Omnibus) repository and will be publicly available after Dec. 1, 2017. Other datasets used in the study (Miyauchi et al., 2016) are available in the GEO repository, GSE85316, GSE14308, GSE30431, GSE92940.

### REFERENCES

Akiba, H., Takeda, K., Kojima, Y., Usui, Y., Harada, N., Yamazaki, T., Ma, J., Tezuka, K., Yagita, H., and Okumura, K. (2005). The role of ICOS in the CXCR5+ follicular B helper T cell maintenance in vivo. J Immunol 175, 2340-2348.

Avogadri, F., Zappasodi, R., Yang, A., Budhu, S., Malandro, N., Hirschhorn-Cymerman, D., Tiwari, S., Maughan, M. F., Olmsted, R., Wolchok, J. D., and Merghoub, T. (2014). Combination of alphavirus replicon particle-based vaccination with immunomodulatory antibodies: therapeutic activity in the B16 melanoma mouse model and immune correlates. Cancer immunology research 2, 448-458.

Ballesteros-Tato, A., Leon, B., Graf, B. A., Moquin, A., Adams, P. S., Lund, F. E., and Randall, T. D. (2012). Interleukin-2 inhibits germinal center formation by limiting T follicular helper cell differentiation. Immunity 36, 847-856.

Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.

Baumjohann, D., Preite, S., Reboldi, A., Ronchi, F., Ansel, K. M., Lanzavecchia, A., and Sallusto, F. (2013). Persistent antigen and germinal center B cells sustain T follicular helper cell responses and phenotype. Immunity 38, 596-605.

Brahmer, J., Reckamp, K. L., Baas, P., Crino, L., Eberhardt, W. E., Poddubskaya, E., Antonia, S., Pluzanski, A., Vokes, E. E., Holgado, E., et al. (2015). Nivolumab versus Docetaxel in Advanced Squamous-Cell Non-Small-Cell Lung Cancer. N Engl J Med 373, 123-135.

Budhu, S., Loike, J. D., Pandolfi, A., Han, S., Catalano, G., Constantinescu, A., Clynes, R., and Silverstein, S. C. (2010). CD8+ T cell concentration determines their efficiency in killing cognate antigen-expressing syngeneic mammalian cells in vitro and in mouse tissues. J Exp Med 207, 223-235.

Callahan, M. K., Horak, C. E., Curran, M. A., Hollman, T., Schaer, D. A., Yuan, J., Lesokhin, A. M., Kitano, S., Hong, Q., Ariyan, C. E., et al. (2013). Peripheral and tumor immune correlates in patients with advanced melanoma treated with combination nivolumab (anti-PD-1, BMS-936558, ONO-4538) and ipilimumab. J Clin Oncol 31, suppl; abstr 3003.

Cardenas, M. G., Yu, W., Beguelin, W., Teater, M. R., Geng, H., Goldstein, R. L., Oswald, E., Hatzi, K., Yang, S. N., Cohen, J., et al. (2016). Rationally designed BCL6 inhibitors target activated B cell diffuse large B cell lymphoma. J Clin Invest 126, 3351-3362.

Cerchietti, L. C., Ghetu, A. F., Zhu, X., Da Silva, G. F., Zhong, S., Matthews, M., Bunting, K. L., Polo, J. M., Fares, C., Arrowsmith, C. H., et al. (2010). A small-molecule inhibitor of BCL6 kills DLBCL cells in vitro and in vivo. Cancer Cell 17, 400-411.

Chen, H., Liakou, C. I., Kamat, A., Pettaway, C., Ward, J. F., Tang, D. N., Sun, J., Jungbluth, A. A., Troncoso, P., Logothetis, C., and Sharma, P. (2009). Anti-CTLA-4 therapy results in higher CD4+ICOShi T cell frequency and IFN-gamma levels in both nonmalignant and malignant prostate tissues. Proc Natl Acad Sci USA 106, 2729-2734.

Choi, Y. S., Gullicksrud, J. A., Xing, S., Zeng, Z., Shan, Q., Li, F., Love, P. E., Peng, W., Xue, H. H., and Crotty, S. (2015). LEF-1 and TCF-1 orchestrate T(FH) differentiation by regulating differentiation circuits upstream of the transcriptional repressor Bcl6. Nature immunology 16, 980-990.

Crotty, S. (2014). T follicular helper cell differentiation, function, and roles in disease. Immunity 41, 529-542.

Cubas, R. A., Mudd, J. C., Savoye, A. L., Perreau, M., van Grevenynghe, J., Metcalf, T., Connick, E., Meditz, A., Freeman, G. J., Abesada-Terk, G., Jr., et al. (2013). Inadequate T follicular cell help impairs B cell immunity during HIV infection. Nat Med 19, 494-499.

Dong, H., Strome, S. E., Salomao, D. R., Tamura, H., Hirano, F., Flies, D. B., Roche, P. C., Lu, J., Zhu, G., Tamada, K., et al. (2002). Tumor-associated B7-H1 promotes T-cell apoptosis: a potential mechanism of immune evasion. Nat Med 8, 793-800.

Ellestad, K. K., Thangavelu, G., Ewen, C. L., Boon, L., and Anderson, C. C. (2014). PD-1 is not required for natural or peripherally induced regulatory T cells: Severe autoimmunity despite normal production of regulatory T cells. Eur J Immunol 44, 3560-3572.

Fife, B. T., and Bluestone, J. A. (2008). Control of peripheral T-cell tolerance and autoimmunity via the CTLA-4 and PD-1 pathways. Immunol Rev 224, 166-182.

Friedman, C. F., Proverbs-Singh, T. A., and Postow, M. A. (2016). Treatment of the Immune-Related Adverse Effects of Immune Checkpoint Inhibitors: A Review. JAMA Oncol 2, 1346-1353.

Gagliani, N., Magnani, C. F., Huber, S., Gianolini, M. E., Pala, M., Licona-Limon, P., Guo, B., Herbert, D. R., Bulfone, A., Trentini, F., et al. (2013). Coexpression of CD49b and LAG-3 identifies human and mouse T regulatory type 1 cells. Nat Med 19, 739-746.

Hale, J. S., and Ahmed, R. (2015). Memory T follicular helper CD4 T cells. Front Immunol 6, 16.

Hanzelmann, S., Castelo, R., and Guinney, J. (2013). GSVA: gene set variation analysis for microarray and RNA-seq data. BMC Bioinformatics 14, 7.

He, J., Tsai, L. M., Leong, Y. A., Hu, X., Ma, C. S., Chevalier, N., Sun, X., Vandenberg, K., Rockman, S., Ding, Y., et al. (2013). Circulating precursor CCR7(lo)PD-1(hi) CXCR5(+) CD4(+) T cells indicate Tfh cell activity and promote antibody responses upon antigen reexposure. Immunity 39, 770-781.

He, R., Hou, S., Liu, C., Zhang, A., Bai, Q., Han, M., Yang, Y., Wei, G., Shen, T., Yang, X., et al. (2016). Follicular CXCR5-expressing CD8+ T-cells curtail chronic viral infection. Nature.

Hellmann, M. D., Rizvi, N. A., Goldman, J. W., Gettinger, S. N., Borghaei, H., Brahmer, J. R., Ready, N. E., Gerber, D. E., Chow, L. Q., Juergens, R. A., et al. (2016). Nivolumab plus ipilimumab as first-line treatment for advanced non-small-cell lung cancer (CheckMate 012): results of an open-label, phase 1, multicohort study. Lancet Oncol.

Hodi, F. S., O'Day, S. J., McDermott, D. F., Weber, R. W., Sosman, J. A., Haanen, J. B., Gonzalez, R., Robert, C., Schadendorf, D., Hassel, J. C., et al. (2010). Improved survival with ipilimumab in patients with metastatic melanoma. N Engl J Med 363, 711-723.

Holmgaard, R. B., Zamarin, D., Li, Y., Gasmi, B., Munn, D. H., Allison, J. P., Merghoub, T., and Wolchok, J. D. (2015). Tumor-Expressed IDO Recruits and Activates MDSCs in a T_{reg}-Dependent Manner. Cell Rep 13, 412-424.

Hou, T. Z., Qureshi, O. S., Wang, C. J., Baker, J., Young, S. P., Walker, L. S., and Sansom, D. M. (2015). A transendocytosis model of CTLA-4 function predicts its suppressive behavior on regulatory T cells. J Immunol 194, 2148-2159.

Im, S. J., Hashimoto, M., Gerner, M. Y., Lee, J., Kissick, H. T., Burger, M. C., Shan, Q., Hale, J. S., Lee, J., Nasti, T. H., et al. (2016). Defining CD8+ T cells that provide the proliferative burst after PD-1 therapy. Nature.

Iwai, Y., Ishida, M., Tanaka, Y., Okazaki, T., Honjo, T., and Minato, N. (2002). Involvement of PD-L1 on tumor cells in the escape from host immune system and tumor immunotherapy by PD-L1 blockade. Proc Natl Acad Sci USA 99, 12293-12297.

Johnston, R. J., Choi, Y. S., Diamond, J. A., Yang, J. A., and Crotty, S. (2012). STATS is a potent negative regulator of T_{FH} cell differentiation. J Exp Med 209, 243-250.

Kageyama, R., Cannons, J. L., Zhao, F., Yusuf, I., Lao, C., Locci, M., Schwartzberg, P. L., and Crotty, S. (2012). The receptor Ly108 functions as a SAP adaptor-dependent on-off switch for T cell help to B cells and NKT cell development. Immunity 36, 986-1002.

Keir, M. E., Butte, M. J., Freeman, G. J., and Sharpe, A. H. (2008). PD-1 and its ligands in tolerance and immunity. Annu Rev Immunol 26, 677-704.

Kenefeck, R., Wang, C. J., Kapadi, T., Wardzinski, L., Attridge, K., Clough, L. E., Heuts, F., Kogimtzis, A., Patel, S., Rosenthal, M., et al. (2015). Follicular helper T cell signature in type 1 diabetes. J Clin Invest 125, 292-303.

Larkin, J., Chiarion-Sileni, V., Gonzalez, R., Grob, J. J., Cowey, C. L., Lao, C. D., Schadendorf, D., Dummer, R., Smylie, M., Rutkowski, P., et al. (2015). Combined Nivolumab and Ipilimumab or Monotherapy in Untreated Melanoma. N Engl J Med 373, 23-34.

Leach, D. R., Krummel, M. F., and Allison, J. P. (1996). Enhancement of antitumor immunity by CTLA-4 blockade. Science 271, 1734-1736.

Liu, X., Yan, X., Zhong, B., Nurieva, R. I., Wang, A., Wang, X., Martin-Orozco, N., Wang, Y., Chang, S. H., Esplugues, E., et al. (2012). Bcl6 expression specifies the T follicular helper cell program in vivo. J Exp Med 209, 1841-1852, S1841-1824.

Liu, Y., Carlsson, R., Comabella, M., Wang, J., Kosicki, M., Carrion, B., Hasan, M., Wu, X., Montalban, X., Dziegiel, M. H., et al. (2014). FoxAl directs the lineage and immunosuppressive properties of a novel regulatory T cell population in EAE and MS. Nat Med 20, 272-282.

Lutsky, J., Antonia, S. J., Blake-Haskins, A. et al. (2014). A phase 1 study of MEDI4736, and anti-PD-L1 antibody, in patients with advanced solid tumors. J Clin Oncol 32, suppl; abstr 3001.

Ma, C. S., Deenick, E. K., Batten, M., and Tangye, S. G. (2012). The origins, function, and regulation of T follicular helper cells. J Exp Med 209, 1241-1253.

Miyauchi, K., Sugimoto-Ishige, A., Harada, Y., Adachi, Y., Usami, Y., Kaji, T., Inoue, K., Hasegawa, H., Watanabe, T., Hijikata, A., et al. (2016). Protective neutralizing influenza antibody response in the absence of T follicular helper cells. Nature immunology 17, 1447-1458.

Murphy, T. L., Tussiwand, R., and Murphy, K. M. (2013). Specificity through cooperation: BATF-IRF interactions control immune-regulatory networks. Nat Rev Immunol 13, 499-509.

Ng Tang, D., Shen, Y., Sun, J., Wen, S., Wolchok, J. D., Yuan, J., Allison, J. P., and Sharma, P. (2013). Increased frequency of ICOS+CD4 T cells as a pharmacodynamic biomarker for anti-CTLA-4 therapy. Cancer immunology research 1, 229-234.

Pentcheva-Hoang, T., Egen, J. G., Wojnoonski, K., and Allison, J. P. (2004). B7-1 and B7-2 selectively recruit CTLA-4 and CD28 to the immunological synapse. Immunity 21, 401-413.

Pollock, P. M., Cohen-Solal, K., Sood, R., Namkoong, J., Martino, J. J., Koganti, A., Zhu, H., Robbins, C., Makalowska, I., Shin, S. S., et al. (2003). Melanoma mouse model implicates metabotropic glutamate signaling in melanocytic neoplasia. Nat Genet 34, 108-112.

Postow, M. A., Chesney, J., Pavlick, A. C., Robert, C., Grossmann, K., McDermott, D., Linette, G. P., Meyer, N., Giguere, J. K., Agarwala, S. S., et al. (2015). Nivolumab and ipilimumab versus ipilimumab in untreated melanoma. N Engl J Med 372, 2006-2017.

Rizvi, N., Gettinger, S. N., Goldman, J., et al. (2015). Safety and efficacy of first-line nivolumab (NIVO: anti-programmed death-1 [PD-1]) and ipilimumab in non-small cell lung cancer (NSCLC). J Thorac Oncol 10, suppl. 2; abstr 786.

Robert, C., Schachter, J., Long, G. V., Arance, A., Grob, J. J., Mortier, L., Daud, A., Carlino, M. S., McNeil, C., Lotem, M., et al. (2015). Pembrolizumab versus Ipilimumab in Advanced Melanoma. N Engl J Med 372, 2521-2532.

Sage, P. T., Alvarez, D., Godec, J., von Andrian, U. H., and Sharpe, A. H. (2014a). Circulating T follicular regulatory and helper cells have memory-like properties. J Clin Invest 124, 5191-5204.

Sage, P. T., Francisco, L. M., Carman, C. V., and Sharpe, A. H. (2013). The receptor PD-1 controls follicular regulatory T cells in the lymph nodes and blood. Nature immunology 14, 152-161.

Sage, P. T., Paterson, A. M., Lovitch, S. B., and Sharpe, A. H. (2014b). The coinhibitory receptor CTLA-4 controls B cell responses by modulating T follicular helper, T follicular regulatory, and T regulatory cells. Immunity 41, 1026-1039.

Sahoo, A., Alekseev, A., Tanaka, K., Obertas, L., Lerman, B., Haymaker, C., Clise-Dwyer, K., McMurray, J. S., and Nurieva, R. (2015). Batf is important for IL-4 expression in T follicular helper cells. Nat Commun 6, 7997.

Seth, S., Ravens, I., Kremmer, E., Maier, M. K., Hadis, U., Hardtke, S., Forster, R., and Bernhardt, G. (2009). Abundance of follicular helper T cells in Peyer's patches is modulated by CD155. Eur J Immunol 39, 3160-3170.

Strome, S. E., Dong, H., Tamura, H., Voss, S. G., Flies, D. B., Tamada, K., Salomao, D., Cheville, J., Hirano, F., Lin, W., et al. (2003). B7-H1 blockade augments adoptive T-cell immunotherapy for squamous cell carcinoma. Cancer Res 63, 6501-6505.

Tangye, S. G., Ma, C. S., Brink, R., and Deenick, E. K. (2013). The good, the bad and the ugly—T_{FH} cells in human health and disease. Nat Rev Immunol 13, 412-426.

Topalian, S. L., Hodi, F. S., Brahmer, J. R., Gettinger, S. N., Smith, D. C., McDermott, D. F., Powderly, J. D., Carvajal, R. D., Sosman, J. A., Atkins, M. B., et al. (2012). Safety, activity, and immune correlates of anti-PD-1 antibody in cancer. N Engl J Med 366, 2443-2454.

Wang, C. J., Heuts, F., Ovcinnikovs, V., Wardzinski, L., Bowers, C., Schmidt, E. M., Kogimtzis, A., Kenefeck, R., Sansom, D. M., and Walker, L. S. (2015). CTLA-4 controls follicular helper T-cell differentiation by regulating the strength of CD28 engagement. Proc Natl Acad Sci USA 112, 524-529.

Weber, J. S., D'Angelo, S. P., Minor, D., Hodi, F. S., Gutzmer, R., Neyns, B., Hoeller, C., Khushalani, N. I., Miller, W. H., Jr., Lao, C. D., et al. (2015). Nivolumab versus chemotherapy in patients with advanced melanoma who progressed after anti-CTLA-4 treatment (CheckMate 037): a randomised, controlled, open-label, phase 3 trial. Lancet Oncol 16, 375-384.

Wherry, E. J., and Kurachi, M. (2015). Molecular and cellular insights into T cell exhaustion. Nat Rev Immunol 15, 486-499.

Wing, J. B., Ise, W., Kurosaki, T., and Sakaguchi, S. (2014). Regulatory T cells control antigen-specific expansion of Tfh cell number and humoral immune responses via the coreceptor CTLA-4. Immunity 41, 1013-1025.

Wing, K., Onishi, Y., Prieto-Martin, P., Yamaguchi, T., Miyara, M., Fehervari, Z., Nomura, T., and Sakaguchi, S. (2008). CTLA-4 control over Foxp3+ regulatory T cell function. Science 322, 271-275.

Wolchok, J. D., Kluger, H., Callahan, M. K., Postow, M. A., Rizvi, N. A., Lesokhin, A. M., Segal, N. H., Ariyan, C. E., Gordon, R. A., Reed, K., et al. (2013). Nivolumab plus ipilimumab in advanced melanoma. N Engl J Med 369, 122-133.

Yarilin, D., Xu, K., Turkekul, M., Fan, N., Romin, Y., Fijisawa, S., Barlas, A., and Manova-Todorova, K. (2015). Machine-based method for multiplex in situ molecular characterization of tissues by immunofluorescence detection. Sci Rep 5, 9534.

Zappasodi, R., and Merghoub, T. (2015). Alphavirus-based vaccines in melanoma: rationale and potential improvements in immunotherapeutic combinations. Immunotherapy 7, 981-997.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The present invention is further described by the following claims.

1. A method of treating cancer in a patient undergoing immune checkpoint blockade (ICB) therapy, the method comprising:
a. measuring 4PD1^{hi} cell frequency in a blood sample from the patient at least about three weeks after a first dose of ICB therapy comprising a first dosage of at least one of a PD-1 inhibitor and a CTLA-4 inhibitor; and
b. administering to the patient a second dose of ICB therapy comprising a second dosage of at least one of a PD-1 inhibitor and a CTLA-4 inhibitor, wherein the dosages of the PD-1 inhibitor and the CTLA-4 inhibitor are adjusted from the first dosage to the second dosage based on the 4PD1^{hi} cell frequency.

2. The method of claim 1, wherein the second dosage of the PD-1 inhibitor is increased as compared to the first dosage if the 4PD1^{hi} cell frequency is high.

3. The method of claim 1, wherein the second dosage of the PD-1 inhibitor is decreased as compared to the first dosage if the 4PD1^{hi} cell frequency is low.

4. The method of claim 1, wherein the second dosage of the CTLA-4 inhibitor is increased as compared to the first dosage if the 4PD1^{hi} cell frequency is low.

5. The method of claim 1, wherein the second dosage of the CTLA-4 inhibitor is decreased as compared to the first dosage if the 4PD1^{hi} cell frequency is high.

6. The method of claim 1, comprising measuring 4PD1^{hi} cell frequency in a blood sample from the patient prior to the first dose of ICB therapy.

7. The method of claim 1, comprising administering to the patient a BCL6 inhibitor.

8. A method for predicting a response to ICB therapy in a cancer patient and treating with ICB therapy the cancer patient, the method comprising:
a. measuring 4PD1^{hi} cell frequency in a blood sample from the cancer patient;
b. classifying the cancer patient as susceptible to ICB therapy wherein the 4PD1^{hi} cell frequency is low or classifying the cancer patient as resistant to ICB therapy wherein the 4PD1^{hi} cell frequency is high; and
c. administering to the cancer patient: a lower dosage of a PD-1 inhibitor and/or a higher dosage of a CTLA-4 inhibitor wherein the patient is susceptible to ICB therapy, or a higher dosage of a PD-1 inhibitor and/or a lower dosage of a CTLA-4 inhibitor wherein the patient is resistant to ICB therapy.

9. An ex vivo method for determining whether a cancer patient is susceptible to ICB therapy comprising a CTLA-4 inhibitor, the method comprising measuring 4PD1^{hi} cell frequency in a blood sample from the cancer patient, wherein a low 4PD1^{hi} cell frequency indicates that the patient is susceptible to ICB therapy comprising a CTLA-4 inhibitor and wherein a high 4PD1^{hi} cell frequency indicates that the patient is resistant to ICB therapy comprising a CTLA-4 inhibitor.

10. A method for in vitro prediction of the probability of a cancer patient responding to ICB therapy comprising a CTLA-4 inhibitor, the method comprising:
a. determining the frequency of 4PD1^{hi} cells in a blood sample from the cancer patient; and
b. comparing the frequency of 4PD1^{hi} cells determined in step (a) with a reference frequency of 4PD1^{hi} cells obtained from cancer patients who have responded to ICB therapy comprising a CTLA-4;
wherein, if the frequency of 4PD1^{hi} cells determined in step (a) is the same as or lower than the reference frequency, it is predicted that the cancer patient will respond to ICB therapy comprising CTLA-4.

11. (canceled)

12. (canceled)

13. (canceled)

14. The method of claim 1, wherein the PD-1 inhibitor is selected from the group consisting of nivolumab, pembrolizumab, pidilizumab, and REGN2810.

15. The method of claim 1, wherein the PD-1 inhibitor is selected from the group consisting of atezolizumab, avelumab, durvalumab, and BMS-936559.

16. The method of claim 1, wherein the CTLA-4 inhibitor is selected from the group consisting of ipilimumab and tremelimumab.

17. The method of claim 1, wherein 4PD1^{hi} cell frequency is measured using immunohistochemistry.

18. The method of claim 1, wherein 4PD1^{hi} cell frequency is measured using flow cytometry.

19. The method of claim 18, wherein the flow cytometry is fluorescence-activated cell sorting (FACS).

20. The method of claim 1, wherein 4PD1^{hi} cell frequency is measured using gene expression signature.
